SEQUENCE LISTING (249 sequences)

SEQ ID NO: 1 - DNA, 19 bp, Artificial Sequence
  synthetic nucleic acid molecule
gcgcatgcagcacctgatt

SEQ ID NO: 2 - DNA, 19 bp, Artificial Sequence
  synthetic nucleic acid molecule
aggcagaggctgccatcta

SEQ ID NO: 3 - DNA, 19 bp, Artificial Sequence
  synthetic nucleic acid molecule
gaggctgccatctaccact

SEQ ID NO: 4 - DNA, 19 bp, Artificial Sequence
  synthetic nucleic acid molecule
catctaccacttgcaatta

SEQ ID NO: 5 - DNA, 19 bp, Artificial Sequence
  synthetic nucleic acid molecule
gcaattatttgaggaactc

SEQ ID NO: 6 - DNA, 19 bp, Artificial Sequence
  synthetic nucleic acid molecule
atttgaggaactccgccgc

SEQ ID NO: 7 - DNA, 19 bp, Artificial Sequence
  synthetic nucleic acid molecule
tggcgcccattaccagcga

SEQ ID NO: 8 - DNA, 19 bp, Artificial Sequence
  synthetic nucleic acid molecule
cccattaccagcgacccca

SEQ ID NO: 9 - DNA, 19 bp, Artificial Sequence
  synthetic nucleic acid molecule
cattaccagcgaccccaca

SEQ ID NO: 10 - DNA, 19 bp, Artificial Sequence
  synthetic nucleic acid molecule
ttaccagcgaccccacaga

SEQ ID NO: 11 - DNA, 19 bp, Artificial Sequence
  synthetic nucleic acid molecule
gccgtggaggcctccttca

SEQ ID NO: 12 - DNA, 19 bp, Artificial Sequence
  synthetic nucleic acid molecule
tgcagtggggccataatcg

SEQ ID NO: 13 - DNA, 19 bp, Artificial Sequence
  synthetic nucleic acid molecule
gccataatcgtcctcacca

SEQ ID NO: 14 - DNA, 19 bp, Artificial Sequence
  synthetic nucleic acid molecule
ccataatcgtcctcaccaa

SEQ ID NO: 15 - RNA, 19 nt, Artificial Sequence
  synthetic nucleic acid molecule
uuccucaaauaauugcaag

SEQ ID NO: 16 - RNA, 21 nt, Artificial Sequence
  synthetic nucleic acid molecule
uuccucaaauaauugcaaguu

SEQ ID NO: 17 - RNA, 19 nt, Artificial Sequence
  synthetic nucleic acid molecule
uuggugaggacgauuaugg

SEQ ID NO: 18 - RNA, 21 nt, Artificial Sequence
  synthetic nucleic acid molecule
uuggugaggacgauuaugguu

SEQ ID NO: 19 - RNA, 19 nt, Artificial Sequence
  synthetic nucleic acid molecule
auggcagccucugccucac

SEQ ID NO: 20 - RNA, 21 nt, Artificial Sequence
  synthetic nucleic acid molecule
auggcagccucugccucacuu

SEQ ID NO: 21 - RNA, 19 nt, Artificial Sequence
  synthetic nucleic acid molecule
aaucaggugcugcaugcgc

SEQ ID NO: 22 - RNA, 21 nt, Artificial Sequence
  synthetic nucleic acid molecule
aaucaggugcugcaugcgcuu

SEQ ID NO: 23 - RNA, 19 nt, Artificial Sequence
  synthetic nucleic acid molecule
uagauggcagccucugccu

SEQ ID NO: 24 - RNA, 21 nt, Artificial Sequence
  synthetic nucleic acid molecule
uagauggcagccucugccuuu

SEQ ID NO: 25 - RNA, 19 nt, Artificial Sequence
  synthetic nucleic acid molecule
agugguagauggcagccuc

SEQ ID NO: 26 - RNA, 21 nt, Artificial Sequence
  synthetic nucleic acid molecule
agugguagauggcagccucuu

SEQ ID NO: 27 - RNA, 19 nt, Artificial Sequence
  synthetic nucleic acid molecule
uaauugcaagugguagaug

SEQ ID NO: 28 - RNA, 21 nt, Artificial Sequence
  synthetic nucleic acid molecule
uaauugcaagugguagauguu

SEQ ID NO: 29 - RNA, 19 nt, Artificial Sequence
  synthetic nucleic acid molecule
gaguuccucaaauaauugc

SEQ ID NO: 30 - RNA, 21 nt, Artificial Sequence
  synthetic nucleic acid molecule
gaguuccucaaauaauugcuu

SEQ ID NO: 31 - RNA, 19 nt, Artificial Sequence
  synthetic nucleic acid molecule
gcggcggaguuccucaaau

SEQ ID NO: 32 - RNA, 21 nt, Artificial Sequence
  synthetic nucleic acid molecule
gcggcggaguuccucaaauuu

SEQ ID NO: 33 - RNA, 19 nt, Artificial Sequence
  synthetic nucleic acid molecule
ucgcugguaaugggcgcca

SEQ ID NO: 34 - RNA, 21 nt, Artificial Sequence
  synthetic nucleic acid molecule
ucgcugguaaugggcgccauu

SEQ ID NO: 35 - RNA, 19 nt, Artificial Sequence
  synthetic nucleic acid molecule
uggggucgcugguaauggg

SEQ ID NO: 36 - RNA, 21 nt, Artificial Sequence
  synthetic nucleic acid molecule
uggggucgcugguaauggguu

SEQ ID NO: 37 - RNA, 19 nt, Artificial Sequence
  synthetic nucleic acid molecule
uguggggucgcugguaaug

SEQ ID NO: 38 - RNA, 21 nt, Artificial Sequence
  synthetic nucleic acid molecule
uguggggucgcugguaauguu

SEQ ID NO: 39 - RNA, 19 nt, Artificial Sequence
  synthetic nucleic acid molecule
ucuguggggucgcugguaa

SEQ ID NO: 40 - RNA, 21 nt, Artificial Sequence
  synthetic nucleic acid molecule
ucuguggggucgcugguaauu

SEQ ID NO: 41 - RNA, 19 nt, Artificial Sequence
  synthetic nucleic acid molecule
ugaaggaggccuccacggc

SEQ ID NO: 42 - RNA, 21 nt, Artificial Sequence
  synthetic nucleic acid molecule
ugaaggaggccuccacggcuu

SEQ ID NO: 43 - RNA, 19 nt, Artificial Sequence
  synthetic nucleic acid molecule
uggugaggacgauuauggc

SEQ ID NO: 44 - RNA, 21 nt, Artificial Sequence
  synthetic nucleic acid molecule
uggugaggacgauuauggcuu

SEQ ID NO: 45 - RNA, 19 nt, Artificial Sequence
  synthetic nucleic acid molecule
uggugaggacgauuauggc

SEQ ID NO: 46 - RNA, 21 nt, Artificial Sequence
  synthetic nucleic acid molecule
uggugaggacgauuauggcuu

SEQ ID NO: 47 - RNA, 19 nt, Artificial Sequence
  synthetic nucleic acid molecule
uuggugaggacgauuaugg

SEQ ID NO: 48 - RNA, 21 nt, Artificial Sequence
  synthetic nucleic acid molecule
uuggugaggacgauuaugguu

SEQ ID NO: 49 - DNA, 160 bp, Homo sapiens
atagctcgtgaggctgaggcagccatgttccaccgcaagctgtttgaagaacttgtgcgagcctcaagtcactccacagacctcatggaagccatggccatgggcagcgtggaggcttcttataagtgtttagcagcagctttgatagttctgacggagt

SEQ ID NO: 50 - DNA, 160 bp, Homo sapiens
attgcccgtgaggcagaggctgccatctaccacttgcaattatttgaggaactccgccgcctggcgcccattaccagcgaccccacagaagccaccgccgtgggtgccgtggaggcctccttcaagtgctgcagtggggccataatcgtcctcaccaagt

SEQ ID NO: 51 - DNA, 2674 bp, Homo sapiens
cgccgcgcttcctcctgaaggtgactgcgcccgcggggacgcagggggcggggcccgggtcgcccggagccgggattgggcagagggcggggcggcggagggattgcggcggcccgcagcgggataaccttgaggctgaggcagtggctccttgcacagcagctgcacgcgccgtggctccggatctcttcgtctttgcagcgtagcccgagtcggtcagcgccggaggtgagcggtgcaggaggctacgccatcagtccccaccaagggccagtcgcccggctagtgcggaatcccggcgcgccggccggccccgggcacgcaggcagggcggcgcaggatccagggcgtctgggatgcagtggagctcagagagaggagaacggctcctcacgcctggggcctgctcttcagaagtccccagcgccgttccttccagatcaggacctcagcagccatgtcgaagccccatagtgaagccgggactgccttcattcagacccagcagctgcacgcagccatggctgacacattcctggagcacatgtgccgcctggacattgattcaccacccatcacagcccggaacactggcatcatctgtaccattggcccagcttcccgatcagtggagacgttgaaggagatgattaagtctggaatgaatgtggctcgtctgaacttctctcatggaactcatgagtaccatgcggagaccatcaagaatgtgcgcacagccacggaaagctttgcttctgaccccatcctctaccggcccgttgctgtggctctagacactaaaggacctgagatccgaactgggctcatcaagggcagcggcactgcagaggtggagctgaagaagggagccactctcaaaatcacgctggataacgcctacatggaaaagtgtgacgagaacatcctgtggctggactacaagaacatctgcaaggtggtggaagtgggcagcaagatctacgtggatgatgggcttatttctctccaggtgaagcagaaaggtgccgacttcctggtgacggaggtggaaaatggtggctccttgggcagcaagaagggtgtgaaccttcctggggctgctgtggacttgcctgctgtgtcggagaaggacatccaggatctgaagtttggggtcgagcaggatgttgatatggtgtttgcgtcattcatccgcaaggcatctgatgtccatgaagttaggaaggtcctgggagagaagggaaagaacatcaagattatcagcaaaatcgagaatcatgagggggttcggaggtttgatgaaatcctggaggccagtgatgggatcatggtggctcgtggtgatctaggcattgagattcctgcagagaaggtcttccttgctcagaagatgatgattggacggtgcaaccgagctgggaagcctgtcatctgtgctactcagatgctggagagcatgatcaagaagccccgccccactcgggctgaaggcagtgatgtggccaatgcagtcctggatggagccgactgcatcatgctgtctggagaaacagccaaaggggactatcctctggaggctgtgcgcatgcagcacctgatagctcgtgaggctgaggcagccatgttccaccgcaagctgtttgaagaacttgtgcgagcctcaagtcactccacagacctcatggaagccatggccatgggcagcgtggaggcttcttataagtgtttagcagcagctttgatagttctgacggagtctggcaggtctgctcaccaggtggccagataccgcccacgtgcccccatcattgctgtgacccggaatccccagacagctcgtcaggcccacctgtaccgtggcatcttccctgtgctgtgcaaggacccagtccaggaggcctgggctgaggacgtggacctccgggtgaactttgccatgaatgttggcaaggcccgaggcttcttcaagaagggagatgtggtcattgtgctgaccggatggcgccctggctccggcttcaccaacaccatgcgtgttgttcctgtgccgtgatggaccccagagcccctcctccagcccctgtcccacccccttcccccagcccatccattaggccagcaacgcttgtagaactcactctgggctgtaacgtggcactggtaggttgggacaccagggaagaagatcaacgcctcactgaaacatggctgtgtttgcagcctgctctagtgggacagcccagagcctggctgcccatcatgtggccccacccaatcaagggaagaaggaggaatgctggactggaggcccctggagccagatggcaagagggtgacagcttcctttcctgtgtgtactctgtccagttcctttagaaaaaatggatgcccagaggactcccaaccctggcttggggtcaagaaacagccagcaagagttaggggccttagggcactgggctgttgttccattgaagccgactctggccctggcccttacttgcttctctagctctctaggcctctccagtttgcacctgtccccaccctccactcagctgtcctgcagcaaacactccaccctccaccttccattttcccccactactgcagcacctccaggcctgttgctatagagcctacctgtatgtcaataaacaacagctgaagcacc

SEQ ID NO: 52 - DNA, 2498 bp, Homo sapiens
cgccgcgcttcctcctgaaggtgactgcgcccgcggggacgcagggggcggggcccgggtcgcccggagccgggattgggcagagggcggggcggcggagggattgcggcggcccgcagcgggataaccttgaggctgaggcagtggctccttgcacagcagctgcacgcgccgtggctccggatctcttcgtctttgcagcgtagcccgagtcggtcagcgccggaggacctcagcagccatgtcgaagccccatagtgaagccgggactgccttcattcagacccagcagctgcacgcagccatggctgacacattcctggagcacatgtgccgcctggacattgattcaccacccatcacagcccggaacactggcatcatctgtaccattggcccagcttcccgatcagtggagacgttgaaggagatgattaagtctggaatgaatgtggctcgtctgaacttctctcatggaactcatgagtaccatgcggagaccatcaagaatgtgcgcacagccacggaaagctttgcttctgaccccatcctctaccggcccgttgctgtggctctagacactaaaggacctgagatccgaactgggctcatcaagggcagcggcactgcagaggtggagctgaagaagggagccactctcaaaatcacgctggataacgcctacatggaaaagtgtgacgagaacatcctgtggctggactacaagaacatctgcaaggtggtggaagtgggcagcaagatctacgtggatgatgggcttatttctctccaggtgaagcagaaaggtgccgacttcctggtgacggaggtggaaaatggtggctccttgggcagcaagaagggtgtgaaccttcctggggctgctgtggacttgcctgctgtgtcggagaaggacatccaggatctgaagtttggggtcgagcaggatgttgatatggtgtttgcgtcattcatccgcaaggcatctgatgtccatgaagttaggaaggtcctgggagagaagggaaagaacatcaagattatcagcaaaatcgagaatcatgagggggttcggaggtttgatgaaatcctggaggccagtgatgggatcatggtggctcgtggtgatctaggcattgagattcctgcagagaaggtcttccttgctcagaagatgatgattggacggtgcaaccgagctgggaagcctgtcatctgtgctactcagatgctggagagcatgatcaagaagccccgccccactcgggctgaaggcagtgatgtggccaatgcagtcctggatggagccgactgcatcatgctgtctggagaaacagccaaaggggactatcctctggaggctgtgcgcatgcagcacctgatagctcgtgaggctgaggcagccatgttccaccgcaagctgtttgaagaacttgtgcgagcctcaagtcactccacagacctcatggaagccatggccatgggcagcgtggaggcttcttataagtgtttagcagcagctttgatagttctgacggagtctggcaggtctgctcaccaggtggccagataccgcccacgtgcccccatcattgctgtgacccggaatccccagacagctcgtcaggcccacctgtaccgtggcatcttccctgtgctgtgcaaggacccagtccaggaggcctgggctgaggacgtggacctccgggtgaactttgccatgaatgttggcaaggcccgaggcttcttcaagaagggagatgtggtcattgtgctgaccggatggcgccctggctccggcttcaccaacaccatgcgtgttgttcctgtgccgtgatggaccccagagcccctcctccagcccctgtcccacccccttcccccagcccatccattaggccagcaacgcttgtagaactcactctgggctgtaacgtggcactggtaggttgggacaccagggaagaagatcaacgcctcactgaaacatggctgtgtttgcagcctgctctagtgggacagcccagagcctggctgcccatcatgtggccccacccaatcaagggaagaaggaggaatgctggactggaggcccctggagccagatggcaagagggtgacagcttcctttcctgtgtgtactctgtccagttcctttagaaaaaatggatgcccagaggactcccaaccctggcttggggtcaagaaacagccagcaagagttaggggccttagggcactgggctgttgttccattgaagccgactctggccctggcccttacttgcttctctagctctctaggcctctccagtttgcacctgtccccaccctccactcagctgtcctgcagcaaacactccaccctccaccttccattttcccccactactgcagcacctccaggcctgttgctatagagcctacctgtatgtcaataaacaacagctgaagcaccaaaaaaaaaaaaaaaaaaaaaaaaaaaaaaaaaaaaaaaa

SEQ ID NO: 53 - protein, 531 aa, Homo sapiens
MSKPHSEAGTAFIQTQQLHAAMADTFLEHMCRLDIDSPPITARNTGIICTIGPASRSVETLKEMIKSGMNVARLNFSHGTHEYHAETIKNVRTATESFASDPILYRPVAVALDTKGPEIRTGLIKGSGTAEVELKKGATLKITLDNAYMEKCDENILWLDYKNICKVVEVGSKIYVDDGLISLQVKQKGADFLVTEVENGGSLGSKKGVNLPGAAVDLPAVSEKDIQDLKFGVEQDVDMVFASFIRKASDVHEVRKVLGEKGKNIKIISKIENHEGVRRFDEILEASDGIMVARGDLGIEIPAEKVFLAQKMMIGRCNRAGKPVICATQMLESMIKKPRPTRAEGSDVANAVLDGADCIMLSGETAKGDYPLEAVRMQHLIAREAEAAMFHRKLFEELVRASSHSTDLMEAMAMGSVEASYKCLAAALIVLTESGRSAHQVARYRPRAPIIAVTRNPQTARQAHLYRGIFPVLCKDPVQEAWAEDVDLRVNFAMNVGKARGFFKKGDVVIVLTGWRPGSGFTNTMRVVPVP

SEQ ID NO: 54 - protein, 531 aa, Homo sapiens; identical to SEQ ID NO: 53

SEQ ID NO: 55 - skipped

SEQ ID NO: 56 - DNA, 2479 bp, Homo sapiens
cgccgcgcttcctcctgaaggtgactgcgcccgcggggacgcagggggcggggcccgggtcgcccggagccgggattgggcagagggcggggcggcggagggattgcggcggcccgcagcgggataaccttgaggctgaggcagtggctccttgcacagcagctgcacgcgccgtggctccggatctcttcgtctttgcagcgtagcccgagtcggtcagcgccggaggacctcagcagccatgtcgaagccccatagtgaagccgggactgccttcattcagacccagcagctgcacgcagccatggctgacacattcctggagcacatgtgccgcctggacattgattcaccacccatcacagcccggaacactggcatcatctgtaccattggcccagcttcccgatcagtggagacgttgaaggagatgattaagtctggaatgaatgtggctcgtctgaacttctctcatggaactcatgagtaccatgcggagaccatcaagaatgtgcgcacagccacggaaagctttgcttctgaccccatcctctaccggcccgttgctgtggctctagacactaaaggacctgagatccgaactgggctcatcaagggcagcggcactgcagaggtggagctgaagaagggagccactctcaaaatcacgctggataacgcctacatggaaaagtgtgacgagaacatcctgtggctggactacaagaacatctgcaaggtggtggaagtgggcagcaagatctacgtggatgatgggcttatttctctccaggtgaagcagaaaggtgccgacttcctggtgacggaggtggaaaatggtggctccttgggcagcaagaagggtgtgaaccttcctggggctgctgtggacttgcctgctgtgtcggagaaggacatccaggatctgaagtttggggtcgagcaggatgttgatatggtgtttgcgtcattcatccgcaaggcatctgatgtccatgaagttaggaaggtcctgggagagaagggaaagaacatcaagattatcagcaaaatcgagaatcatgagggggttcggaggtttgatgaaatcctggaggccagtgatgggatcatggtggctcgtggtgatctaggcattgagattcctgcagagaaggtcttccttgctcagaagatgatgattggacggtgcaaccgagctgggaagcctgtcatctgtgctactcagatgctggagagcatgatcaagaagccccgccccactcgggctgaaggcagtgatgtggccaatgcagtcctggatggagccgactgcatcatgctgtctggagaaacagccaaaggggactatcctctggaggctgtgcgcatgcagcacctgattgcccgtgaggcagaggctgccatctaccacttgcaattatttgaggaactccgccgcctggcgcccattaccagcgaccccacagaagccaccgccgtgggtgccgtggaggcctccttcaagtgctgcagtggggccataatcgtcctcaccaagtctggcaggtctgctcaccaggtggccagataccgcccacgtgcccccatcattgctgtgacccggaatccccagacagctcgtcaggcccacctgtaccgtggcatcttccctgtgctgtgcaaggacccagtccaggaggcctgggctgaggacgtggacctccgggtgaactttgccatgaatgttggcaaggcccgaggcttcttcaagaagggagatgtggtcattgtgctgaccggatggcgccctggctccggcttcaccaacaccatgcgtgttgttcctgtgccgtgatggaccccagagcccctcctccagcccctgtcccacccccttcccccagcccatccattaggccagcaacgcttgtagaactcactctgggctgtaacgtggcactggtaggttgggacaccagggaagaagatcaacgcctcactgaaacatggctgtgtttgcagcctgctctagtgggacagcccagagcctggctgcccatcatgtggccccacccaatcaagggaagaaggaggaatgctggactggaggcccctggagccagatggcaagagggtgacagcttcctttcctgtgtgtactctgtccagttcctttagaaaaaatggatgcccagaggactcccaaccctggcttggggtcaagaaacagccagcaagagttaggggccttagggcactgggctgttgttccattgaagccgactctggccctggcccttacttgcttctctagctctctaggcctctccagtttgcacctgtccccaccctccactcagctgtcctgcagcaaacactccaccctccaccttccattttcccccactactgcagcacctccaggcctgttgctatagagcctacctgtatgtcaataaacaacagctgaagcaccaaaaaaaaaaaaaaaaaaaaa

SEQ ID NO: 57 - protein, 531 aa, Homo sapiens
MSKPHSEAGTAFIQTQQLHAAMADTFLEHMCRLDIDSPPITARNTGIICTIGPASRSVETLKEMIKSGMNVARLNFSHGTHEYHAETIKNVRTATESFASDPILYRPVAVALDTKGPEIRTGLIKGSGTAEVELKKGATLKITLDNAYMEKCDENILWLDYKNICKVVEVGSKIYVDDGLISLQVKQKGADFLVTEVENGGSLGSKKGVNLPGAAVDLPAVSEKDIQDLKFGVEQDVDMVFASFIRKASDVHEVRKVLGEKGKNIKIISKIENHEGVRRFDEILEASDGIMVARGDLGIEIPAEKVFLAQKMMIGRCNRAGKPVICATQMLESMIKKPRPTRAEGSDVANAVLDGADCIMLSGETAKGDYPLEAVRMQHLIAREAEAAIYHLQLFEELRRLAPITSDPTEATAVGAVEASFKCCSGAIIVLTKSGRSAHQVARYRPRAPIIAVTRNPQTARQAHLYRGIFPVLCKDPVQEAWAEDVDLRVNFAMNVGKARGFFKKGDVVIVLTGWRPGSGFTNTMRVVPVP

SEQ ID NO: 58 - protein, 574 aa, Homo sapiens
MSIQENISSLQLRSWVSKSQRDLAKSILIGAPGGPAGYLRRASVAQLTQELGTAFFQQQQLPAAMADTFLEHLCLLDIDSEPVAARSTSIIATIGPASRSVERLKEMIKAGMNIARLNFSHGSHEYHAESIANVREAVESFAGSPLSYRPVAIALDTKGPEIRTGILQGGPESEVELVKGSQVLVTVDPAFRTRGNANTVWVDYPNIVRVVPVGGRIYIDDGLISLVVQKIGPEGLVTQVENGGVLGSRKGVNLPGAQVDLPGLSEQDVRDLRFGVEHGVDIVFASFVRKASDVAAVRAALGPEGHGIKIISKIENHEGVKRFDEILEVSDGIMVARGDLGIEIPAEKVFLAQKMMIGRCNLAGKPVVCATQMLESMITKPRPTRAETSDVANAVLDGADCIMLSGETAKGNFPVEAVKMQHAIAREAEAAVYHRQLFEELRRAAPLSRDPTEVTAIGAVEAAFKCCAAAIIVLTTTGRSAQLLSRYRPRAAVIAVTRSAQAARQVHLCRGVFPLLYREPPEAIWADDVDRRVQFGIESGKLRGFLRVGDLVIVVTGWRPGSGYTNIMRVLSIS

SEQ ID NO: 59 - protein, 543 aa, Homo sapiens
MEGPAGYLRRASVAQLTQELGTAFFQQQQLPAAMADTFLEHLCLLDIDSEPVAARSTSIIATIGPASRSVERLKEMIKAGMNIARLNFSHGSHEYHAESIANVREAVESFAGSPLSYRPVAIALDTKGPEIRTGILQGGPESEVELVKGSQVLVTVDPAFRTRGNANTVWVDYPNIVRVVPVGGRIYIDDGLISLVVQKIGPEGLVTQVENGGVLGSRKGVNLPGAQVDLPGLSEQDVRDLRFGVEHGVDIVFASFVRKASDVAAVRAALGPEGHGIKIISKIENHEGVKRFDEILEVSDGIMVARGDLGIEIPAEKVFLAQKMMIGRCNLAGKPVVCATQMLESMITKPRPTRAETSDVANAVLDGADCIMLSGETAKGNFPVEAVKMQHAIAREAEAAVYHRQLFEELRRAAPLSRDPTEVTAIGAVEAAFKCCAAAIIVLTTTGRSAQLLSRYRPRAAVIAVTRSAQAARQVHLCRGVFPLLYREPPEAIWADDVDRRVQFGIESGKLRGFLRVGDLVIVVTGWRPGSGYTNIMRVLSIS

SEQ ID NO: 60 - RNA, 19 nt, Artificial Sequence
  synthetic nucleic acid molecule
gccauaaucguccucacca

SEQ ID NO: 61 - DNA, 19 bp, Artificial Sequence
  synthetic nucleic acid molecule
cttacgctgagtacttcga

SEQ ID NO: 62 - DNA, 20 bp, Artificial Sequence
  synthetic nucleic acid molecule
cattgattcaccacccatca

SEQ ID NO: 63 - DNA, 20 bp, Artificial Sequence
  synthetic nucleic acid molecule
agacgagccacattcattcc

SEQ ID NO: 64 - DNA, 20 bp, Artificial Sequence
  synthetic nucleic acid molecule
cgagcctcaagtcactccac

SEQ ID NO: 65 - DNA, 20 bp, Artificial Sequence
  synthetic nucleic acid molecule
gtgagcagacctgccagact

SEQ ID NO: 66 - DNA, 24 bp, Artificial Sequence
  synthetic nucleic acid molecule
attatttgaggaactccgccgcct

SEQ ID NO: 67 - DNA, 23 bp, Artificial Sequence
  synthetic nucleic acid molecule
attccgggtcacagcaatgatgg

SEQ ID NO: 68 - RNA, 19 nt, Artificial Sequence
  synthetic nucleic acid molecule
gcgcaugcagcaccugauu

SEQ ID NO: 69 - RNA, 19 nt, Artificial Sequence
  synthetic nucleic acid molecule
cgcaugcagcaccugauug

SEQ ID NO: 70 - RNA, 19 nt, Artificial Sequence
  synthetic nucleic acid molecule
gcaugcagcaccugauugc

SEQ ID NO: 71 - RNA, 19 nt, Artificial Sequence
  synthetic nucleic acid molecule
caugcagcaccugauugcc

SEQ ID NO: 72 - RNA, 19 nt, Artificial Sequence
  synthetic nucleic acid molecule
augcagcaccugauugccc

SEQ ID NO: 73 - RNA, 19 nt, Artificial Sequence
  synthetic nucleic acid molecule
ugcagcaccugauugcccg

SEQ ID NO: 74 - RNA, 19 nt, Artificial Sequence
  synthetic nucleic acid molecule
gcagcaccugauugcccgu

SEQ ID NO: 75 - RNA, 19 nt, Artificial Sequence
  synthetic nucleic acid molecule
cagcaccugauugcccgug

SEQ ID NO: 76 - RNA, 19 nt, Artificial Sequence
  synthetic nucleic acid molecule
agcaccugauugcccguga

SEQ ID NO: 77 - RNA, 19 nt, Artificial Sequence
  synthetic nucleic acid molecule
gcaccugauugcccgugag

SEQ ID NO: 78 - RNA, 19 nt, Artificial Sequence
  synthetic nucleic acid molecule
caccugauugcccgugagg

SEQ ID NO: 79 - RNA, 19 nt, Artificial Sequence
  synthetic nucleic acid molecule
accugauugcccgugaggc

SEQ ID NO: 80 - RNA, 19 nt, Artificial Sequence
  synthetic nucleic acid molecule
ccugauugcccgugaggca

SEQ ID NO: 81 - RNA, 19 nt, Artificial Sequence
  synthetic nucleic acid molecule
cugauugcccgugaggcag

SEQ ID NO: 82 - RNA, 19 nt, Artificial Sequence
  synthetic nucleic acid molecule
ugauugcccgugaggcaga

SEQ ID NO: 83 - RNA, 19 nt, Artificial Sequence
  synthetic nucleic acid molecule
gauugcccgugaggcagag

SEQ ID NO: 84 - RNA, 19 nt, Artificial Sequence
  synthetic nucleic acid molecule
auugcccgugaggcagagg

SEQ ID NO: 85 - RNA, 19 nt, Artificial Sequence
  synthetic nucleic acid molecule
uugcccgugaggcagaggc

SEQ ID NO: 86 - RNA, 19 nt, Artificial Sequence
  synthetic nucleic acid molecule
ugcccgugaggcagaggcu

SEQ ID NO: 87 - RNA, 19 nt, Artificial Sequence
  synthetic nucleic acid molecule
gcccgugaggcagaggcug

SEQ ID NO: 88 - RNA, 19 nt, Artificial Sequence
  synthetic nucleic acid molecule
cccgugaggcagaggcugc

SEQ ID NO: 89 - RNA, 19 nt, Artificial Sequence
  synthetic nucleic acid molecule
ccgugaggcagaggcugcc

SEQ ID NO: 90 - RNA, 19 nt, Artificial Sequence
  synthetic nucleic acid molecule
cgugaggcagaggcugcca

SEQ ID NO: 91 - RNA, 19 nt, Artificial Sequence
  synthetic nucleic acid molecule
gugaggcagaggcugccau

SEQ ID NO: 92 - RNA, 19 nt, Artificial Sequence
  synthetic nucleic acid molecule
ugaggcagaggcugccauc

SEQ ID NO: 93 - RNA, 19 nt, Artificial Sequence
  synthetic nucleic acid molecule
gaggcagaggcugccaucu

SEQ ID NO: 94 - RNA, 19 nt, Artificial Sequence
  synthetic nucleic acid molecule
aggcagaggcugccaucua

SEQ ID NO: 95 - RNA, 19 nt, Artificial Sequence
  synthetic nucleic acid molecule
ggcagaggcugccaucuac

SEQ ID NO: 96 - RNA, 19 nt, Artificial Sequence
  synthetic nucleic acid molecule
gcagaggcugccaucuacc

SEQ ID NO: 97 - RNA, 19 nt, Artificial Sequence
  synthetic nucleic acid molecule
cagaggcugccaucuacca

SEQ ID NO: 98 - RNA, 19 nt, Artificial Sequence
  synthetic nucleic acid molecule
agaggcugccaucuaccac

SEQ ID NO: 99 - RNA, 19 nt, Artificial Sequence
  synthetic nucleic acid molecule
gaggcugccaucuaccacu

SEQ ID NO: 100 - RNA, 19 nt, Artificial Sequence
  synthetic nucleic acid molecule
aggcugccaucuaccacuu

SEQ ID NO: 101 - RNA, 19 nt, Artificial Sequence
  synthetic nucleic acid molecule
ggcugccaucuaccacuug

SEQ ID NO: 102 - RNA, 19 nt, Artificial Sequence
  synthetic nucleic acid molecule
gcugccaucuaccacuugc

SEQ ID NO: 103 - RNA, 19 nt, Artificial Sequence
  synthetic nucleic acid molecule
cugccaucuaccacuugca

SEQ ID NO: 104 - RNA, 19 nt, Artificial Sequence
  synthetic nucleic acid molecule
ugccaucuaccacuugcaa

SEQ ID NO: 105 - RNA, 19 nt, Artificial Sequence
  synthetic nucleic acid molecule
gccaucuaccacuugcaau

SEQ ID NO: 106 - RNA, 19 nt, Artificial Sequence
  synthetic nucleic acid molecule
ccaucuaccacuugcaauu

SEQ ID NO: 107 - RNA, 19 nt, Artificial Sequence
  synthetic nucleic acid molecule
caucuaccacuugcaauua

SEQ ID NO: 108 - RNA, 19 nt, Artificial Sequence
  synthetic nucleic acid molecule
aucuaccacuugcaauuau

SEQ ID NO: 109 - RNA, 19 nt, Artificial Sequence
  synthetic nucleic acid molecule
ucuaccacuugcaauuauu

SEQ ID NO: 110 - RNA, 19 nt, Artificial Sequence
  synthetic nucleic acid molecule
cuaccacuugcaauuauuu

SEQ ID NO: 111 - RNA, 19 nt, Artificial Sequence
  synthetic nucleic acid molecule
uaccacuugcaauuauuug

SEQ ID NO: 112 - RNA, 19 nt, Artificial Sequence
  synthetic nucleic acid molecule
accacuugcaauuauuuga

SEQ ID NO: 113 - RNA, 19 nt, Artificial Sequence
  synthetic nucleic acid molecule
ccacuugcaauuauuugag

SEQ ID NO: 114 - RNA, 19 nt, Artificial Sequence
  synthetic nucleic acid molecule
cacuugcaauuauuugagg

SEQ ID NO: 115 - RNA, 19 nt, Artificial Sequence
  synthetic nucleic acid molecule
acuugcaauuauuugagga

SEQ ID NO: 116 - RNA, 19 nt, Artificial Sequence
  synthetic nucleic acid molecule
cuugcaauuauuugaggaa

SEQ ID NO: 117 - RNA, 19 nt, Artificial Sequence
  synthetic nucleic acid molecule
uugcaauuauuugaggaac

SEQ ID NO: 118 - RNA, 19 nt, Artificial Sequence
  synthetic nucleic acid molecule
ugcaauuauuugaggaacu

SEQ ID NO: 119 - RNA, 19 nt, Artificial Sequence
  synthetic nucleic acid molecule
gcaauuauuugaggaacuc

SEQ ID NO: 120 - RNA, 19 nt, Artificial Sequence
  synthetic nucleic acid molecule
caauuauuugaggaacucc

SEQ ID NO: 121 - RNA, 19 nt, Artificial Sequence
  synthetic nucleic acid molecule
aauuauuugaggaacuccg

SEQ ID NO: 122 - RNA, 19 nt, Artificial Sequence
  synthetic nucleic acid molecule
auuauuugaggaacuccgc

SEQ ID NO: 123 - RNA, 19 nt, Artificial Sequence
  synthetic nucleic acid molecule
uuauuugaggaacuccgcc

SEQ ID NO: 124 - RNA, 19 nt, Artificial Sequence
  synthetic nucleic acid molecule
uauuugaggaacuccgccg

SEQ ID NO: 125 - RNA, 19 nt, Artificial Sequence
  synthetic nucleic acid molecule
auuugaggaacuccgccgc

SEQ ID NO: 126 - RNA, 19 nt, Artificial Sequence
  synthetic nucleic acid molecule
uuugaggaacuccgccgcc

SEQ ID NO: 127 - RNA, 19 nt, Artificial Sequence
  synthetic nucleic acid molecule
uugaggaacuccgccgccu

SEQ ID NO: 128 - RNA, 19 nt, Artificial Sequence
  synthetic nucleic acid molecule
ugaggaacuccgccgccug

SEQ ID NO: 129 - RNA, 19 nt, Artificial Sequence
  synthetic nucleic acid molecule
gaggaacuccgccgccugg

SEQ ID NO: 130 - RNA, 19 nt, Artificial Sequence
  synthetic nucleic acid molecule
aggaacuccgccgccuggc

SEQ ID NO: 131 - RNA, 19 nt, Artificial Sequence
  synthetic nucleic acid molecule
ggaacuccgccgccuggcg

SEQ ID NO: 132 - RNA, 19 nt, Artificial Sequence
  synthetic nucleic acid molecule
gaacuccgccgccuggcgc

SEQ ID NO: 133 - RNA, 19 nt, Artificial Sequence
  synthetic nucleic acid molecule
aacuccgccgccuggcgcc

SEQ ID NO: 134 - RNA, 19 nt, Artificial Sequence
  synthetic nucleic acid molecule
acuccgccgccuggcgccc

SEQ ID NO: 135 - RNA, 19 nt, Artificial Sequence
  synthetic nucleic acid molecule
cuccgccgccuggcgccca

SEQ ID NO: 136 - RNA, 19 nt, Artificial Sequence
  synthetic nucleic acid molecule
uccgccgccuggcgcccau

SEQ ID NO: 137 - RNA, 19 nt, Artificial Sequence
  synthetic nucleic acid molecule
ccgccgccuggcgcccauu

SEQ ID NO: 138 - RNA, 19 nt, Artificial Sequence
  synthetic nucleic acid molecule
cgccgccuggcgcccauua

SEQ ID NO: 139 - RNA, 19 nt, Artificial Sequence
  synthetic nucleic acid molecule
gccgccuggcgcccauuac

SEQ ID NO: 140 - RNA, 19 nt, Artificial Sequence
  synthetic nucleic acid molecule
ccgccuggcgcccauuacc

SEQ ID NO: 141 - RNA, 19 nt, Artificial Sequence
  synthetic nucleic acid molecule
cgccuggcgcccauuacca

SEQ ID NO: 142 - RNA, 19 nt, Artificial Sequence
  synthetic nucleic acid molecule
gccuggcgcccauuaccag

SEQ ID NO: 143 - RNA, 19 nt, Artificial Sequence
  synthetic nucleic acid molecule
ccuggcgcccauuaccagc

SEQ ID NO: 144 - RNA, 19 nt, Artificial Sequence
  synthetic nucleic acid molecule
cuggcgcccauuaccagcg

SEQ ID NO: 145 - RNA, 19 nt, Artificial Sequence
  synthetic nucleic acid molecule
uggcgcccauuaccagcga

SEQ ID NO: 146 - RNA, 19 nt, Artificial Sequence
  synthetic nucleic acid molecule
ggcgcccauuaccagcgac

SEQ ID NO: 147 - RNA, 19 nt, Artificial Sequence
  synthetic nucleic acid molecule
gcgcccauuaccagcgacc

SEQ ID NO: 148 - RNA, 19 nt, Artificial Sequence
  synthetic nucleic acid molecule
cgcccauuaccagcgaccc

SEQ ID NO: 149 - RNA, 19 nt, Artificial Sequence
  synthetic nucleic acid molecule
gcccauuaccagcgacccc

SEQ ID NO: 150 - RNA, 19 nt, Artificial Sequence
  synthetic nucleic acid molecule
cccauuaccagcgacccca

SEQ ID NO: 151 - RNA, 19 nt, Artificial Sequence
  synthetic nucleic acid molecule
ccauuaccagcgaccccac

SEQ ID NO: 152 - RNA, 19 nt, Artificial Sequence
  synthetic nucleic acid molecule
cauuaccagcgaccccaca

SEQ ID NO: 153 - RNA, 19 nt, Artificial Sequence
  synthetic nucleic acid molecule
auuaccagcgaccccacag

SEQ ID NO: 154 - RNA, 19 nt, Artificial Sequence
  synthetic nucleic acid molecule
uuaccagcgaccccacaga

SEQ ID NO: 155 - RNA, 19 nt, Artificial Sequence
  synthetic nucleic acid molecule
uaccagcgaccccacagaa

SEQ ID NO: 156 - RNA, 19 nt, Artificial Sequence
  synthetic nucleic acid molecule
accagcgaccccacagaag

SEQ ID NO: 157 - RNA, 19 nt, Artificial Sequence
  synthetic nucleic acid molecule
ccagcgaccccacagaagc

SEQ ID NO: 158 - RNA, 19 nt, Artificial Sequence
  synthetic nucleic acid molecule
cagcgaccccacagaagcc

SEQ ID NO: 159 - RNA, 19 nt, Artificial Sequence
  synthetic nucleic acid molecule
agcgaccccacagaagcca

SEQ ID NO: 160 - RNA, 19 nt, Artificial Sequence
  synthetic nucleic acid molecule
gcgaccccacagaagccac

SEQ ID NO: 161 - RNA, 19 nt, Artificial Sequence
  synthetic nucleic acid molecule
cgaccccacagaagccacc

SEQ ID NO: 162 - RNA, 19 nt, Artificial Sequence
  synthetic nucleic acid molecule
gaccccacagaagccaccg

SEQ ID NO: 163 - RNA, 19 nt, Artificial Sequence
  synthetic nucleic acid molecule
accccacagaagccaccgc

SEQ ID NO: 164 - RNA, 19 nt, Artificial Sequence
  synthetic nucleic acid molecule
ccccacagaagccaccgcc

SEQ ID NO: 165 - RNA, 19 nt, Artificial Sequence
  synthetic nucleic acid molecule
cccacagaagccaccgccg

SEQ ID NO: 166 - RNA, 19 nt, Artificial Sequence
  synthetic nucleic acid molecule
ccacagaagccaccgccgu

SEQ ID NO: 167 - RNA, 19 nt, Artificial Sequence
  synthetic nucleic acid molecule
cacagaagccaccgccgug

SEQ ID NO: 168 - RNA, 19 nt, Artificial Sequence
  synthetic nucleic acid molecule
acagaagccaccgccgugg

SEQ ID NO: 169 - RNA, 19 nt, Artificial Sequence
  synthetic nucleic acid molecule
cagaagccaccgccguggg

SEQ ID NO: 170 - RNA, 19 nt, Artificial Sequence
  synthetic nucleic acid molecule
agaagccaccgccgugggu

SEQ ID NO: 171 - RNA, 19 nt, Artificial Sequence
  synthetic nucleic acid molecule
gaagccaccgccgugggug

SEQ ID NO: 172 - RNA, 19 nt, Artificial Sequence
  synthetic nucleic acid molecule
aagccaccgccgugggugc

SEQ ID NO: 173 - RNA, 19 nt, Artificial Sequence
  synthetic nucleic acid molecule
agccaccgccgugggugcc

SEQ ID NO: 174 - RNA, 19 nt, Artificial Sequence
  synthetic nucleic acid molecule
gccaccgccgugggugccg

SEQ ID NO: 175 - RNA, 19 nt, Artificial Sequence
  synthetic nucleic acid molecule
ccaccgccgugggugccgu

SEQ ID NO: 176 - RNA, 19 nt, Artificial Sequence
  synthetic nucleic acid molecule
caccgccgugggugccgug

SEQ ID NO: 177 - RNA, 19 nt, Artificial Sequence
  synthetic nucleic acid molecule
accgccgugggugccgugg

SEQ ID NO: 178 - RNA, 19 nt, Artificial Sequence
  synthetic nucleic acid molecule
ccgccgugggugccgugga

SEQ ID NO: 179 - RNA, 19 nt, Artificial Sequence
  synthetic nucleic acid molecule
cgccgugggugccguggag

SEQ ID NO: 180 - RNA, 19 nt, Artificial Sequence
  synthetic nucleic acid molecule
gccgugggugccguggagg

SEQ ID NO: 181 - RNA, 19 nt, Artificial Sequence
  synthetic nucleic acid molecule
ccgugggugccguggaggc

SEQ ID NO: 182 - RNA, 19 nt, Artificial Sequence
  synthetic nucleic acid molecule
cgugggugccguggaggcc

SEQ ID NO: 183 - RNA, 19 nt, Artificial Sequence
  synthetic nucleic acid molecule
gugggugccguggaggccu

SEQ ID NO: 184 - RNA, 19 nt, Artificial Sequence
  synthetic nucleic acid molecule
ugggugccguggaggccuc

SEQ ID NO: 185 - RNA, 19 nt, Artificial Sequence
  synthetic nucleic acid molecule
gggugccguggaggccucc

SEQ ID NO: 186 - RNA, 19 nt, Artificial Sequence
  synthetic nucleic acid molecule
ggugccguggaggccuccu

SEQ ID NO: 187 - RNA, 19 nt, Artificial Sequence
  synthetic nucleic acid molecule
gugccguggaggccuccuu

SEQ ID NO: 188 - RNA, 19 nt, Artificial Sequence
  synthetic nucleic acid molecule
ugccguggaggccuccuuc

SEQ ID NO: 189 - RNA, 19 nt, Artificial Sequence
  synthetic nucleic acid molecule
gccguggaggccuccuuca

SEQ ID NO: 190 - RNA, 19 nt, Artificial Sequence
  synthetic nucleic acid molecule
ccguggaggccuccuucaa

SEQ ID NO: 191 - RNA, 19 nt, Artificial Sequence
  synthetic nucleic acid molecule
cguggaggccuccuucaag

SEQ ID NO: 192 - RNA, 19 nt, Artificial Sequence
  synthetic nucleic acid molecule
guggaggccuccuucaagu

SEQ ID NO: 193 - RNA, 19 nt, Artificial Sequence
  synthetic nucleic acid molecule
uggaggccuccuucaagug

SEQ ID NO: 194 - RNA, 19 nt, Artificial Sequence
  synthetic nucleic acid molecule
ggaggccuccuucaagugc

SEQ ID NO: 195 - RNA, 19 nt, Artificial Sequence
  synthetic nucleic acid molecule
gaggccuccuucaagugcu

SEQ ID NO: 196 - RNA, 19 nt, Artificial Sequence
  synthetic nucleic acid molecule
aggccuccuucaagugcug

SEQ ID NO: 197 - RNA, 19 nt, Artificial Sequence
  synthetic nucleic acid molecule
ggccuccuucaagugcugc

SEQ ID NO: 198 - RNA, 19 nt, Artificial Sequence
  synthetic nucleic acid molecule
gccuccuucaagugcugca

SEQ ID NO: 199 - RNA, 19 nt, Artificial Sequence
  synthetic nucleic acid molecule
ccuccuucaagugcugcag

SEQ ID NO: 200 - RNA, 19 nt, Artificial Sequence
  synthetic nucleic acid molecule
cuccuucaagugcugcagu

SEQ ID NO: 201 - RNA, 19 nt, Artificial Sequence
  synthetic nucleic acid molecule
uccuucaagugcugcagug

SEQ ID NO: 202 - RNA, 19 nt, Artificial Sequence
  synthetic nucleic acid molecule
ccuucaagugcugcagugg

SEQ ID NO: 203 - RNA, 19 nt, Artificial Sequence
  synthetic nucleic acid molecule
cuucaagugcugcaguggg

SEQ ID NO: 204 - RNA, 19 nt, Artificial Sequence
  synthetic nucleic acid molecule
uucaagugcugcagugggg

SEQ ID NO: 205 - RNA, 19 nt, Artificial Sequence
  synthetic nucleic acid molecule
ucaagugcugcaguggggc

SEQ ID NO: 206 - RNA, 19 nt, Artificial Sequence
  synthetic nucleic acid molecule
caagugcugcaguggggcc

SEQ ID NO: 207 - RNA, 19 nt, Artificial Sequence
  synthetic nucleic acid molecule
aagugcugcaguggggcca

SEQ ID NO: 208 - RNA, 19 nt, Artificial Sequence
  synthetic nucleic acid molecule
agugcugcaguggggccau

SEQ ID NO: 209 - RNA, 19 nt, Artificial Sequence
  synthetic nucleic acid molecule
gugcugcaguggggccaua

SEQ ID NO: 210 - RNA, 19 nt, Artificial Sequence
  synthetic nucleic acid molecule
ugcugcaguggggccauaa

SEQ ID NO: 211 - RNA, 19 nt, Artificial Sequence
  synthetic nucleic acid molecule
gcugcaguggggccauaau

SEQ ID NO: 212 - RNA, 19 nt, Artificial Sequence
  synthetic nucleic acid molecule
cugcaguggggccauaauc

SEQ ID NO: 213 - RNA, 19 nt, Artificial Sequence
  synthetic nucleic acid molecule
ugcaguggggccauaaucg

SEQ ID NO: 214 - RNA, 19 nt, Artificial Sequence
  synthetic nucleic acid molecule
gcaguggggccauaaucgu

SEQ ID NO: 215 - RNA, 19 nt, Artificial Sequence
  synthetic nucleic acid molecule
caguggggccauaaucguc

SEQ ID NO: 216 - RNA, 19 nt, Artificial Sequence
  synthetic nucleic acid molecule
aguggggccauaaucgucc

SEQ ID NO: 217 - RNA, 19 nt, Artificial Sequence
  synthetic nucleic acid molecule
guggggccauaaucguccu

SEQ ID NO: 218 - RNA, 19 nt, Artificial Sequence
  synthetic nucleic acid molecule
uggggccauaaucguccuc

SEQ ID NO: 219 - RNA, 19 nt, Artificial Sequence
  synthetic nucleic acid molecule
ggggccauaaucguccuca

SEQ ID NO: 220 - RNA, 19 nt, Artificial Sequence
  synthetic nucleic acid molecule
gggccauaaucguccucac

SEQ ID NO: 221 - RNA, 19 nt, Artificial Sequence
  synthetic nucleic acid molecule
ggccauaaucguccucacc

SEQ ID NO: 222 - RNA, 19 nt, Artificial Sequence
  synthetic nucleic acid molecule
gccauaaucguccucacca

SEQ ID NO: 223 - RNA, 19 nt, Artificial Sequence
  synthetic nucleic acid molecule
ccauaaucguccucaccaa

SEQ ID NO: 224 - RNA, 19 nt, Artificial Sequence
  synthetic nucleic acid molecule
cauaaucguccucaccaag

SEQ ID NO: 225 - RNA, 19 nt, Artificial Sequence
  synthetic nucleic acid molecule
auaaucguccucaccaagu

SEQ ID NO: 226 - RNA, 19 nt, Artificial Sequence
  synthetic nucleic acid molecule
uaaucguccucaccaaguc

SEQ ID NO: 227 - RNA, 19 nt, Artificial Sequence
  synthetic nucleic acid molecule
aaucguccucaccaagucu

SEQ ID NO: 228 - RNA, 19 nt, Artificial Sequence
  synthetic nucleic acid molecule
aucguccucaccaagucug

SEQ ID NO: 229 - RNA, 19 nt, Artificial Sequence
  synthetic nucleic acid molecule
ucguccucaccaagucugg

SEQ ID NO: 230 - RNA, 19 nt, Artificial Sequence
  synthetic nucleic acid molecule
cguccucaccaagucuggc

SEQ ID NO: 231 - RNA, 19 nt, Artificial Sequence
  synthetic nucleic acid molecule
guccucaccaagucuggca

SEQ ID NO: 232 - RNA, 19 nt, Artificial Sequence
  synthetic nucleic acid molecule
uccucaccaagucuggcag

SEQ ID NO: 233 - RNA, 19 nt, Artificial Sequence
  synthetic nucleic acid molecule
ccucaccaagucuggcagg

SEQ ID NO: 234 - RNA, 19 nt, Artificial Sequence
  synthetic nucleic acid molecule
cucaccaagucuggcaggu

SEQ ID NO: 235 - RNA, 19 nt, Artificial Sequence
  synthetic nucleic acid molecule
ucaccaagucuggcagguc

SEQ ID NO: 236 - RNA, 19 nt, Artificial Sequence
  synthetic nucleic acid molecule
caccaagucuggcaggucu

SEQ ID NO: 237 - RNA, 19 nt, Artificial Sequence
  synthetic nucleic acid molecule
accaagucuggcaggucug

SEQ ID NO: 238 - RNA, 19 nt, Artificial Sequence
  synthetic nucleic acid molecule
ccaagucuggcaggucugc

SEQ ID NO: 239 - RNA, 19 nt, Artificial Sequence
  synthetic nucleic acid molecule
caagucuggcaggucugcu

SEQ ID NO: 240 - RNA, 19 nt, Artificial Sequence
  synthetic nucleic acid molecule
aagucuggcaggucugcuc

SEQ ID NO: 241 - RNA, 19 nt, Artificial Sequence
  synthetic nucleic acid molecule
cuuacgcugaguacuucga

SEQ ID NO: 242 - RNA, 19 nt, Artificial Sequence
  synthetic nucleic acid molecule
ggaccugagauccgaacug

SEQ ID NO: 243 - DNA, 19 bp, Artificial Sequence
  synthetic nucleic acid molecule
cttgcaattatttgaggaa

SEQ ID NO: 244 - DNA, 19 bp, Artificial Sequence
  synthetic nucleic acid molecule
tgcaattatttgaggaact

SEQ ID NO: 245 - DNA, 19 bp, Artificial Sequence
  synthetic nucleic acid molecule
gcagtggggccataatcgt

SEQ ID NO: 246 - DNA, 19 bp, Artificial Sequence
  synthetic nucleic acid molecule
gtgaggcagaggctgccat

SEQ ID NO: 247 - DNA, 19 bp, Artificial Sequence
  synthetic nucleic acid molecule
tctaccacttgcaattatt

SEQ ID NO: 248 - DNA, 19 bp, Artificial Sequence
  synthetic nucleic acid molecule
ggagatgattaagtctgga

SEQ ID NO: 249 - DNA, 19 bp, Artificial Sequence
  synthetic nucleic acid molecule
ggacctgagatccgaactg

(19) United States
(12) Patent Application Publication
(10) Pub. No.: US 2013/0018086 A1
(43) Pub. Date: Jan. 17, 2013

(54) SIRNAS TARGETING EXON 10 OF PYRUVATE KINASE M2

(71) Applicant: Michael Solomon Goldberg, Cambridge, MA (US)

(72) Inventor: Michael Solomon Goldberg, Cambridge, MA (US)

(73) Assignee: Massachusette Institute of Technology, Cambridge, MA (US)

(21) Appl. No.: 13/634,789

(22) PCT Filed: Mar. 10, 2011
(86) PCT No.: PCT/US11/27886

§ 371 Date: Sep. 13, 2012

Related U.S. Application Data
(60) Provisional application No. 61/314,551, filed on Mar. 16, 2010.

(51) Int. Cl. A61K 31/7105; C12N 5/07; A61P 35/04; C12N 15/113

(52) U.S. Cl. 514/44 A; 536/24.5; 435/375

(57) ABSTRACT

The invention relates to nucleic acid molecules and compositions for specific post-transcriptional inhibition of PKM2 expression. Methods for specific inhibition of PKM2 expression in a target cell, for example a cancer cell, are also provided.

### RELATED APPLICATIONS

This application claims priority under 35 U.S.C. §119(e) to United States provisional patent application, U.S. Ser. No. 61/314,551, filed Mar. 16, 2010, the entire contents of which are incorporated herein by reference.

### GOVERNMENT SUPPORT

This work was supported by grants U54 CA119349 from the National Cancer Institute. The U.S. government has certain rights in this invention.

### FIELD OF THE INVENTION

Some aspects of this invention provide nucleic acid molecules and compositions for specific post-transcriptional inhibition of PKM2 expression. Methods for specific inhibition of PKM2 expression in a target cell, for example a cancer cell, are also provided.

### BACKGROUND OF THE INVENTION

Malignant cells exhibit a metabolic state different from that of non-malignant somatic cells, characterized by elevated glucose uptake and reduced rates of oxidative phosphorylation. The altered metabolic state of tumor cells results in elevated lactate production by tumor cells in the presence of oxygen, a phenomenon known as “aerobic glycolysis” or “Warburg effect”.

It has been demonstrated that expression of the M2 isoform of the glycolytic enzyme pyruvate kinase (PKM2) is necessary for the altered metabolic state found in tumor cells. Pyruvate kinase regulates a rate-limiting step in glycolysis and is normally expressed in four isoforms: red blood cells and liver cells express the R and L isoforms, respectively; the M1 isoform is expressed in most somatic cells; and the M2 isoform is expressed during embryonic development. It has been suggested that reexpression of PKM2 in somatic cells might itself not be the transforming event, but the resulting metabolic environment may be a prerequisite for tumor cell proliferation.L

It has been reported that malignant cells exclusively express the M2 isoform and that ablation of PKM2 expression in malignant cells results in decreased cell viability and proliferation.

While it is broadly accepted that PKM2 expression is necessary for malignant cell proliferation, the molecular mechanism effecting the switch from oxidative phosphorylation to aerobic glycolysis is not clearly understood. PKM2 has been reported to be a low-activity enzyme that relies on allosteric activation by the upstream metabolite fructose-1,6-bisphosphate, while PKM1 is constitutively active. Based on this evidence, a feedback loop regulation has been suggested in which downregulation of PKM2 leads to reduced levels of fructose-1,6-bisphosphate resulting in a stabilized shutdown of oxidative phosphorylation. An alternative explanation has been suggested to be that PKM1 efficiently shuttles pyruvate to mitochondria, while PKM2 preferably shuttles pyruvate to lactate dehydrogenase, thus channeling substrate away from oxidative phosphorylation and into the generation of lactate.

### SUMMARY

In some aspects, the present invention provides nucleic acid molecules, nucleic acid constructs, compositions, and methods related to inhibition of PKM2 gene expression in malignant cells or cell populations and/or inhibition of proliferation and/or reduction of cell viability in malignant cells or cell populations. The nucleic acid molecules, nucleic acid constructs, compositions, and methods provided by some aspects of this invention are useful for research and clinical/therapeutic applications.

The present invention, in some aspects, is directed to a nucleic acid molecule useful for inhibiting PKM2 gene expression, also referred to herein as a PKM2-inhibitory nucleic acid molecule. In some aspects, the invention is directed to a PKM2-inhibitory nucleic acid molecule useful for specifically inhibiting expression of PKM2. In some aspects, the invention is directed to a PKM2-inhibitory nucleic acid molecule useful for inhibiting expression of PKM2 while not substantially affecting expression of another PK isoform, for example PKM1. In some aspects, the invention is directed to a nucleic acid molecule useful for an inhibition of proliferation and/or a reduction in viability of a cell or cell population expressing or suspected to express PKM2. In some aspects, the invention is directed to a nucleic acid molecule useful for an inhibition of proliferation and/or a reduction in viability of a malignant cell or cell population. In some aspects, the invention is directed to a nucleic acid molecule useful for an inhibition of proliferation and/or a reduction in viability of a malignant cell or cell population.

In some aspects, the invention is directed to a composition comprising a nucleic acid molecule provided by the invention. In some aspects, the invention is directed to a pharmaceutical composition comprising a nucleic acid molecule provided by the invention. In some aspects, the invention is directed to a use of a nucleic acid molecule provided by the invention in the manufacture of a medicament. In some aspects, the invention is directed to a use of a nucleic acid molecule provided by the invention in the manufacture of a medicament for the treatment of a proliferative disease, for example a cancer.

In some aspects, the invention is directed to a method of contacting a cell expressing or suspected to express PKM2 with a nucleic acid molecule provided by the invention. In some aspects, the invention is directed to a method of contacting a cell expressing or suspected to express PKM2 with a nucleic acid molecule provided by the invention to inhibit expression of PKM2. In some aspects, the invention is directed to a method of contacting a cell expressing or suspected to express PKM2 with a nucleic acid molecule provided by the invention to inhibit proliferation and/or decrease cell viability of the cell and/or its progeny.

In some aspects, the invention is directed to a method of treating a subject for a disease characterized by over expression of a target gene. In some aspects, the invention is directed to a method of treating a subject for a disease characterized by overexpression of PKM2. In some aspects, the invention is directed to a method of treating a subject for a cancer characterized or suspected to be characterized by overexpression of PKM2.

Accordingly, in some embodiments, a nucleic acid molecule inhibiting gene expression is provided. In some embodiments, a nucleic acid molecule inhibiting expression of PKM2 is provided. In some embodiments, a nucleic acid molecule specifically inhibiting expression of PKM2 is provided. In some embodiments, a nucleic acid molecule inhibiting expression of PKM2 while not substantially affecting expression of another PK isoform, for example PKM1, is provided. In some embodiments, a nucleic acid molecule inhibiting of proliferation and/or reducing viability of a cell or cell population expressing or suspected to express PKM2 is provided. In some embodiments, a nucleic acid molecule inhibiting proliferation and/or reducing viability of a malignant cell or cell population is provided. In some embodiments, a nucleic acid molecule inhibiting proliferation and/or a reducing viability of a malignant cell or cell population is provided.

In some embodiments a PKM2 inhibitory nucleic acid is provided that targets a sequence of PKM2 with at least one nucleotide mismatch as compared to different PKM isoforms, for example, PKM1. In some embodiments, the PKM2 inhibitory nucleic acid targets a sequence that comprises or consists of one of the following sequences:

(SEQ ID NO: 1)
5′-GCGCATGCAGCACCTGATT-3′
(SEQ ID NO: 2)
5′-AGGCAGAGGCTGCCATCTA-3′
(SEQ ID NO: 3)
5′-GAGGCTGCCATCTACCACT-3′
(SEQ ID NO: 4)
5′-CATCTACCACTTGCAATTA-3′
(SEQ ID NO: 5)
5′-GCAATTATTTGAGGAACTC-3′
(SEQ ID NO: 6)
5′-ATTTGAGGAACTCCGCCGC-3′
(SEQ ID NO: 7)
5′-TGGCGCCCATTACCAGCGA-3′
(SEQ ID NO: 8)
5′-CCCATTACCAGCGACCCCA-3′
(SEQ ID NO: 9)
5′-CATTACCAGCGACCCCACA-3′
(SEQ ID NO: 10)
5′-TTACCAGCGACCCCACAGA-3′
(SEQ ID NO: 11)
5′-GCCGTGGAGGCCTCCTTCA-3′
(SEQ ID NO: 12)
5′-TGCAGTGGGGCCATAATCG-3′
(SEQ ID NO: 13)
5′-GCCATAATCGTCCTCACCA-3′
(SEQ ID NO: 14)
5′-CCATAATCGTCCTCACCAA-3′

In some embodiments, a PKM2 inhibitory sequence that targets a given DNA sequence also targets a related RNA sequence, for example, the same sequence as the DNA sequence, but consisting of ribonucleotides instead of deoxyribonucleotides and with U substituted for T residues.

In some embodiments, the nucleic acid molecule provided comprises or consists of one of the following nucleotide sequences:

(SEQ ID NO: 15)
5′-UUCCUCAAAUAAUUGCAAG-3′
(SEQ ID NO: 16)
5′-UUCCUCAAAUAAUUGCAAGUU-3′
(SEQ ID NO: 17)
5′-UUGGUGAGGACGAUUAUGG-3′
(SEQ ID NO: 18)
5′-UUGGUGAGGACGAUUAUGGUU-3′
(SEQ ID NO: 19)
5′-AUGGCAGCCUCUGCCUCAC-3′
(SEQ ID NO: 20)
5′-AUGGCAGCCUCUGCCUCACUU-3′
(SEQ ID NO: 21)
5′-AAUCAGGUGCUGCAUGCGC-3′
(SEQ ID NO: 22)
5′-AAUCAGGUGCUGCAUGCGCUU-3′
(SEQ ID NO: 23)
5′-UAGAUGGCAGCCUCUGCCU-3′
(SEQ ID NO: 24)
5′-UAGAUGGCAGCCUCUGCCUUU-3′
(SEQ ID NO: 25)
5′-AGUGGUAGAUGGCAGCCUC-3′
(SEQ ID NO: 26)
5′-AGUGGUAGAUGGCAGCCUCUU-3′
(SEQ ID NO: 27)
5′-UAAUUGCAAGUGGUAGAUG-3′
(SEQ ID NO: 28)
5′-UAAUUGCAAGUGGUAGAUGUU-3′
(SEQ ID NO: 29)
5′-GAGUUCCUCAAAUAAUUGC-3′
(SEQ ID NO: 30)
5′-GAGUUCCUCAAAUAAUUGCUU-3′

(SEQ ID NO: 31)
5′-GCGGCGGAGUUCCUCAAAU-3′
(SEQ ID NO: 32)
5′-GCGGCGGAGUUCCUCAAAUUU-3′
(SEQ ID NO: 33)
5′-UCGCUGGUAAUGGGCGCCA-3′
(SEQ ID NO: 34)
5′-UCGCUGGUAAUGGGCGCCAUU-3′
(SEQ ID NO: 35)
5′-UGGGGUCGCUGGUAAUGGG-3′
(SEQ ID NO: 36)
5′-UGGGGUCGCUGGUAAUGGGUU-3′
(SEQ ID NO: 37)
5′-UGUGGGGUCGCUGGUAAUG-3′
(SEQ ID NO: 38)
5′-UGUGGGGUCGCUGGUAAUGUU-3′
(SEQ ID NO: 39)
5′-UCUGUGGGGUCGCUGGUAA-3′
(SEQ ID NO: 40)
5′-UCUGUGGGGUCGCUGGUAAUU-3′
(SEQ ID NO: 41)
5′-UGAAGGAGGCCUCCACGGC-3′
(SEQ ID NO: 42)
5′-UGAAGGAGGCCUCCACGGCUU-3′
(SEQ ID NO: 43)
5′-UGGUGAGGACGAUUAUGGC-3′
(SEQ ID NO: 44)
5′-UGGUGAGGACGAUUAUGGCUU-3′
(SEQ ID NO: 45)
5′-UGGUGAGGACGAUUAUGGC-3′
(SEQ ID NO: 46)
5′-UGGUGAGGACGAUUAUGGCUU-3′
(SEQ ID NO: 47)
5′-UUGGUGAGGACGAUUAUGG-3′
(SEQ ID NO: 48)
5′-UUGGUGAGGACGAUUAUGGUU-3′

In some embodiments, the nucleic acid molecule is a ribonucleic acid molecule. In some embodiments, the nucleic acid molecule is a deoxyribonucleic acid molecule. In some embodiments, the nucleic acid molecule comprises a modified nucleotide, for example a nucleotide comprising a base other than A, T, C, G, or U. In some embodiments, the nucleic acid molecule comprises a backbone modification, for example a non-hydrolysable nucleotide-nucleotide bond. In some embodiments, the nucleic acid molecule comprises other modifications.

In some embodiments, a composition comprising a nucleic acid molecule comprising or having the structure provided in any of SEQ ID NOs 15-48 is provided. In some embodiments, a RNA duplex comprising an antisense strand as provided in any of SEQ ID NOs 15-48 and a sense strand that is complementary to the antisense strand is provided. In some embodiments, a composition comprising a nucleic acid molecule complementary to any of the structures given in SEQ ID NOs 1-14 is provided.

In some embodiments, a small interfering RNA (siRNA) specifically targeting isoform M2 of pyruvate kinase (PKM2) is provided, the siRNA comprising a duplex stem region, wherein the duplex stem region comprises a nucleotide sequence corresponding to a target sequence of exon 10 of pyruvate kinase (PK), and wherein the target sequence is selected from the group consisting of the structures given in SEQ ID NOs 1-14. In some embodiments, the siRNA does not substantially inhibit the expression of isoform M1 of pyruvate kinase (PKM1). In some embodiments, the pyruvate kinase (PK) is human PK. In some embodiments, the siRNA target sequence is between 12 and 40 nucleotides long. In some embodiments, a pharmaceutical composition comprising a siRNA described herein is provided.

In some embodiments, a method of decreasing expression of PKM2 in a cell is provided. In some embodiments, the method comprises contacting a cell expressing PKM2 with a siRNA specifically targeting PKM2. In some embodiments, the siRNA does not substantially inhibit the expression of PKM1. In some embodiments, the PK is human PK. In some embodiments, the siRNA targets a sequence within exon 10 of PK. In some embodiments, the siRNA target sequence is between 12 and 40 nucleotides long. In some embodiments, the siRNA target sequence is 19 nucleotides long. In some embodiments, the siRNA comprises a nucleotide sequence corresponding to a target sequence selected from the group consisting of the structures given in SEQ ID NOs 1-14.

In some embodiments, a method of treating a subject having a tumor expressing PKM2 or suspected to express PKM2 is provided. In some embodiments, the method comprises administering to the subject having the tumor a siRNA specifically targeting PKM2. In some embodiments, the siRNA does not substantially inhibit the expression of PKM1. In some embodiments, the PK is human PK. In some embodiments, the siRNA targets a sequence within exon 10 of PK. In some embodiments, the siRNA target sequence is between 12 and 40 nucleotides long. In some embodiments, the siRNA target sequence is 19 nucleotides long. In some embodiments, the siRNA comprises a nucleotide sequence corresponding to a target sequence selected from the group consisting of the structures given in SEQ ID NOs 1-14.

These and other aspects and embodiments of the invention, as well as various advantages and utilities will be more apparent with respect to the drawings and detailed description of the invention.

### BRIEF DESCRIPTION OF THE DRAWINGS

The figures are illustrative only and are not required for enablement of the invention disclosed herein.

FIG. 1. The specific and potent knockdown of the M2 isoform of pyruvate kinase by si1 and si2 is confirmed by real-time PCR. Primers were designed to amplify total PK or PKM1 only or PKM2 only for HepG2, SKOV3, A549, and KB cells. Duplicate biological samples were collected 48 h after transfection with 5 nM siRNA and assayed in technical triplicate.

FIG. 2. Immunoblotting of total PKM and of PKM2 in HepG2 and SKOV3 lysate. si1 and si2 specifically knock down the M2 isoform at the protein level. Duplicate biological samples were collected 48 h after transfection with 5 nM siRNA.

FIG. 3. si6 induces the greatest specific knockdown of PKM2 mRNA. si1, si5 also induce potent and specific knockdown. As expected, si7 and si8 do not confer M2-specific knockdown.

FIG. 4. si1 yielded a significant effect on cell viability in HepG2 (a), and si5 had a significant effect on cell viability in SKOV3 (b). Along with si3, si7 and si8 (the two positive controls that target sequences that are common to both isoforms) also decrease proliferation, though none of these induces apoptosis (at 96 h) to the same extent as si1 or si5 in HepG2 (c) and SKOV3 (d), respectively.

FIG. 5. Administration of lipidoid-formulated si1 results in a reduction in tumor volume, eradicating established tumors entirely in more than half of the treated animals after less than three weeks.

FIG. 6. Transfection of si1 or si2 results in decreased cell viability relative to siControl in both HepG2 and SKOV3.

FIG. 7. Real-time PCR of RNA collected from tumors that could be extracted confirm knockdown of the M2 isoform (SKOV3) or compensatory upregulation of the M1 isoform (HepG2).

FIG. 8. Cell viability and apoptosis after transfection of siRNAs in four cell lines.

FIG. 9. Relative viability of HCT116 cells after six days for each member of the siPKM2 library. Cells were transfected with 5 nM siRNA on days 0, 2, 4. Transfections were performed in technical quadruplicate on two separate occasions. Viability was determined using CellTiterGlo® reagent. NT, no treatment.

FIG. 10. The specific and potent knockdown of the M2 isoform of pyruvate kinase by si27, si155, and si156 is examined by real-time PCR. Primers were designed to amplify total PK or PKM1 only or PKM2 only from HCT116 total RNA. Duplicate biological samples were collected 48 h after transfection with 5 nM siRNA and assayed in technical triplicate on two separate occasions. Error bars denote standard deviation.

FIG. 11. Viability and apoptosis of HCT116, HepG2, and SKOV3 cells after six days for the three most active siRNA sequences and controls. Cells were transfected with 5 nM siRNA at 0 h, 48 h, and 96 h. Transfections were performed in technical triplicate on two separate occasions. Viability and apoptosis were determined using the ApotoxGlo™ kit. Error bars denote standard deviation.

FIG. 12. Immunoblotting of total PKM and of PKM2 in HepG2 and SKOV3 lysate. si156 specifically knocks down the M2 isoform at the protein level. Duplicate biological samples were collected 48 h after transfection with 5 nM siRNA. NT, no treatment. Vinculin was used as a loading control.

FIG. 13. Administration of lipidoid-formulated si156 results in a reduction in tumor volume, eradicating established tumors entirely in more than half of the treated animals after less than three weeks. The size difference between the treatment and control groups is significant by ANOVA at the 95% confidence interval. a) HepG2, b) SKOV3. Error bars denote s.e.m. (n=4).

FIG. 14. The specific and potent knockdown of the M2 isoform of pyruvate kinase by si156 is examined by real-time PCR. Primers were designed to amplify total PK or PKM1 only or PKM2 only from HepG2, SKOV3, A549, or KB total RNA. Duplicate biological samples were collected 48 h after transfection with 5 nM siRNA, and each sample was assayed in technical triplicate. NT, no treatment.

FIG. 15. The specific and potent knockdown of the M2 isoform of pyruvate kinase by si156 is confirmed by real-time PCR. Primers were designed to amplify total PK or PKM1 only or PKM2 only from HepG2, SKOV3, A549, or KB total RNA. Duplicate biological samples were collected 48 h after transfection with 5 nM siRNA, and each sample was assayed in technical triplicate. NT, no treatment.

FIG. 16. Real-time PCR of RNA collected from the extracted SKOV3 tumor confirms specific knockdown of the M2 isoform. Interestingly, there appears to be compensatory upregulation of the M1 isoform. Error bars denote standard deviation.

FIG. 17. si156 does not reduce the viability of SF372 adult skin fibroblasts. Transfections were performed in technical quadruplicate on two separate occasions. NT, no treatment. Error bars denote standard deviation.

### DETAILED DESCRIPTION

In some instances, an ideal treatment for cancer is one which would i) target a molecule that is expressed by cancer cells but not by healthy cells, ii) target a molecule that is common to all cancers, iii) target only that molecule, and iv) induce cell death rather than simply decrease proliferation. The specific knockdown of the M2 isoform of pyruvate kinase, the reactivation of which in cancer cells results in aerobic glycolysis and confers a selective growth advantage is demonstrated herein for the first time, according to some aspects of the invention. siRNAs designed to target mismatches between the M2 and M1 isoforms are identified herein. It is also demonstrated according to the invention that such siRNAs confer knockdown of the former but not the latter at the mRNA level and the protein level. The data described herein demonstrate that siRNA directed against PKM2 results in decreased cell viability in multiple cell lines but that, interestingly, the extent of cell death does not directly correlate with knockdown and is seemingly sequence dependent. It is also demonstrated that knockdown of PKM2 results in caspase-mediated apoptosis and that siPKM2 results in substantial tumor regression in xenografts of human cancer cell lines, with complete regression in more than half of the animals.

While much effort in the field of oncology has focused on canonical oncogenes and tumor suppresor genes, the importance of cancer cell metabolism is growing in appreciation. Pyruvate kinase catalyzes the conversion of phosphoenolpyruvate (PEP) to pyruvate and ATP, thereby regulating carbon flux in the cell. Here, we demonstrate the specific knockdown of the M2 isoform of pyruvate kinase, whose normal expression pattern is restricted to embryonic tissue but is reactivated in cancer cells.

In addition to the liver (L) and red blood cell (R) isoforms, two isoforms of pyruvate kinase are found in mammals: M1, which is expressed in adult tissues, and M2, which is expressed in embryonic tissue and tumours³. PKM1 and PKM2 differ by only 23 amino acids within a 56-residue alternatively-spliced exon (9 or 10, respectively)⁴. These amino acids constitute an allosteric pocket unique to PKM2 that allows it to bind its activator, fructose 1,6-bisphosphate (FBP)⁵.

PKM2 is important for the shift in cellular metabolism to aerobic glycolysis, which promotes tumorigenesis, and its expression provides a selective growth advantage for tumor cells in vivo⁶. Known as the Warburg effect⁷, this reprogramming of metabolic gene expression enables cells to suppress apoptotic signaling, to grow in hypoxic environments, and to utilize more glucose for anabolic processes rather than oxidative phosphorylation even when oxygen is not limiting⁸.

Pyruvate kinase activity can be modulated not only by alternative splicing but also by allostery. PKM2 exists as either an active tetramer and an inactive dimer. The latter form is found predominantly in tumor cells⁹, and this conformation is favored by phophotyrosinated oncogenic growth factors, which displace FBP, the glycolytic metabolite that induces formation of the tetrameric form¹⁰. The low activity of the dimer form causes an accumulation of PEP and other glycolytic intermediates, resulting in the redirection of glucose to serve as a carbon source for macromolecular biosynthesis, which is important for cell division.

RNA interference (RNAi) represents a potential revolution in the realm of therapeutics, enabling the drugging of previously-intractable targets in a highly-specific manner¹⁴. It has been discovered that the knockdown of pyruvate kinase by shRNA targeting sequences common to both M1 and M2 isoforms resulted in decreased rates of glucose metabolism and reduced cell proliferation⁶. The ability to target PKM2 specifically is important because it presumably causes knockdown in cancer cells without the same effect on healthy cells, which only express PKM1. To this end, we designed siRNAs that are specific to PKM2 and examined their ability to confer specific knockdown, to decrease cell proliferation, to induce apoptosis, and to eradicate tumor xenografts.

Some aspects of this invention relate to decreasing or inhibiting, also referred to as “knocking down”, PKM2 expression in somatic cells via a cellular RNAi pathway. Some aspects of the invention provide nucleic acid molecules able to inhibit PKM2 expression. Some aspects of the invention provide compositions comprising such PKM2-inhibitory nucleic acid molecules.

Some aspects of the invention provide methods of decreasing PKM2 expression in a target cell. Some aspects of the invention provide methods of decreasing proliferation, reduction of cell viability, and/or induction of apoptosis in a target cell by contacting the cell with a PKM2-inhibitory nucleic acid molecule as provided herein. Some aspects of the invention provide methods for administering a nucleic acid targeted at PKM2 to a subject. Some aspects of this invention provide methods for treating a subject, for example a subject having a tumor expressing PKM2, by administering a nucleic acid molecule, or a composition, provided herein.

### PKM2 Target Sequences

Some aspects of this invention provide nucleic acid molecules that target PKM2 and are useful for decreasing PKM2 expression in vitro and/or in vivo. In some embodiments, nucleic acid molecules are provided that comprise an antisense strand to a target PKM2 coding sequence. In some embodiments, the target PKM2 coding sequence is a sequence comprised in exon 10 of human PK. Exon 10 of human PK is known in the art and described herein. In some embodiments, the target PKM2 sequence is a sequence of exon 10 of human PK that is not identical to a coding sequence of PKM1.

The nucleic acid molecule may be a single-stranded or double stranded nucleic acid molecule. For example at a minimum the nucleic acid molecule may be an antisense sequence corresponding to a target sequence comprised, at least partially, in exon 10 of the human pyruvate kinase gene. Alternatively, the nucleic acid molecule may be a double-stranded nucleic acid molecule that includes a sense and an antisense strand corresponding to a target sequence comprised, at least in part, in exon 10 of the human PK gene. In some embodiments, the antisense strand comprises a nucleotide sequence that is complementary to a nucleotide sequence of exon 10 of the human pyruvate kinase gene. Although the antisense, or guide sequence may be substantially identical to at least a portion of the target sequence, the sequence need not be perfectly identical to be useful, for example, to inhibit expression of PKM2. Without wishing to be bound by theory, it is believed that higher homology of guide and target sequence results in stronger, and more specific knockdown while less homology results in weaker, less specific knockdown.

In some embodiments, the sense strand comprises a nucleotide sequence identical to a nucleotide sequence of exon 10 of the human pyruvate kinase gene. In some embodiments, the target sequence comprises a minimum of a 15 nucleotide sequence within any of the sequences given in SEQ ID NOs 1-14. In some embodiments, the target sequence is the sequence 5′-GCGCATGCAGCACCTGATT-3′ (SEQ ID NO: 1), 5′-AGGCAGAGGCTGCCATCTA-3′, (SEQ ID NO: 2), 5′-GAGGCTGCCATCTACCACT-3′, (SEQ ID NO: 3), 5′-CATCTACCACTTGCAATTA-3′ (SEQ ID NO: 4), 5′-GCAATTATTTGAGGAACTC-3′ (SEQ ID NO: 5), 5′-ATTTGAGGAACTCCGCCGC-3′ (SEQ ID NO: 6), 5′-TGGCGCCCATTACCAGCGA-3′ (SEQ ID NO: 7), 5′-CCCATTACCAGCGACCCCA-3′ (SEQ ID NO: 8), 5′-CATTACCAGCGACCCCACA-3′(SEQ ID NO: 9), 5′-TTACCAGCGACCCCACAGA-3′ (SEQ ID NO: 10), 5′-GCCGTGGAGGCCTCCTTCA-3′ (SEQ ID NO: 11), 5′-TGCAGTGGGGCCATAATCG-3′ (SEQ ID NO: 12), 5′-GCCATAATCGTCCTCACCA-3′ (SEQ ID NO: 13), or 5′-CCATAATCGTCCTCACCAA-3′ (SEQ ID NO: 14).

Given below is an alignment of a representative sequence of exon 9 of human PK, as found in the coding sequence for the M1 isoform, and a representative sequence of exon 10 of human PK, as found in the coding sequence for the M2 isoform. Non-identical nucleotides are highlighted in the exon 10 sequence, and target sequences to which siRNA27, siRNA155 correspond are underlined. The target sequence to which si156 corresponds (given in SEQ ID NO: 14) is a target sequence one nucleotide downstream from the si155 target sequence.

M1: >gi|33286419|ref|NM_{—}182470.1| M1-isoform, exon 9 of human PK
M2: >gi|33286417|ref|NM_{—}002654.3| M2-isoform, exon 10 of human PK

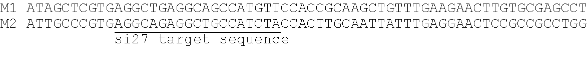

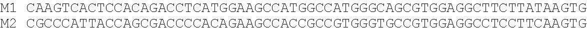

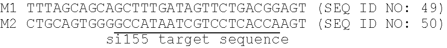

In some embodiments, the target sequence is a sequence comprised, at least partially, in exon 10 of the human pyruvate kinase gene, and comprises at least 1, at least 2, at least 3, at least 4, at least 5, at least 6, at least 7, at least 8, at least 9, at least 10, least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, or at least 39 nucleotide mismatches as compared to the corresponding sequence of exon 9 of the human pyruvate kinase gene. For example, the target sequence of si1, as described in Table 4, comprises 1 nucleotide mismatch, the target sequence of si27, as described herein, comprises 4 nucleotide mismatches, the target sequence of si155, as described herein, comprises 8 nucleotide mismatches, and the target sequence of si156, as described herein, comprises 8 nucleotide mismatches as compared to the corresponding sequence of exon 9.

As used herein, the term “pyruvate kinase” (“PK”) refers to an enzyme, catalyzing the transfer of a phosphate group from phosphoenolpyruvate to adenosine diphosphate, resulting in ATP and pyruvate. The reaction catalyzed by PK is believed to be the rate-limiting step in glycolysis. In humans, PK is expressed in four isoforms. The amino acid sequence of the four isoforms, the genomic sequence of the PK gene, and the coding sequence of each isoform is known in the art and representative human protein and nucleotide sequences are provided below. In the nucleotide sequences, the differentially spliced exon sequence is underlined. In the amino acid sequences, the sequence encoded by the differentially expressed exon is underlined.

TABLE 1
M1 isoform-related sequences:

| TRANSCRIPT | |
|---|---|
| TYPE | SEQUENCE |
| gi\|33286419\|ref\| | CGCCGCGCTTCCTCCTGAAGGTGACTGCGCCCGCGGGGACGCAGGGGGCGGGG |
| NM_182470.1\| | CCCGGGTCGCCCGGAGCCGGGATTGGGCAGAGGGCGGGGCGGCGGAGGGATT |
| Homo sapiens | GCGGCGGCCCGCAGCGGGATAACCTTGAGGCTGAGGCAGTGGCTCCTTGCACA |
| pyruvate kinase, | GCAGCTGCACGCGCCGTGGCTCCGGATCTCTTCGTCTTTGCAGCGTAGCCCGAG |
| muscle (PKM2), | TCGGTCAGCGCCGGAGGTGAGCGGTGCAGGAGGCTACGCCATCAGTCCCCACC |
| transcript variant | AAGGGCCAGTCGCCCGGCTAGTGCGGAATCCCGGCGCGCCGGCCGGCCCCGGG |
| 2, mRNA. | CACGCAGGCAGGGCGGCGCAGGATCCAGGGCGTCTGGGATGCAGTGGAGCTC |
| Transcript | AGAGAGAGGAGAACGGCTCCTCACGCCTGGGGCCTGCTCTTCAGAAGTCCCCA |
| Variant: This | GCGCCGTTCCTTCCAGATCAGGACCTCAGCAGCCATGTCGAAGCCCCATAGTG |
| variant (2) | AAGCCGGGACTGCCTTCATTCAGACCCAGCAGCTGCACGCAGCCATGGCTGAC |
| encodes an | ACATTCCTGGAGCACATGTGCCGCCTGGACATTGATTCACCACCCATCACAGCC |
| isoform (M1) that | CGGAACACTGGCATCATCTGTACCATTGGCCCAGCTTCCCGATCAGTGGAGAC |
| is the same length | GTTGAAGGAGATGATTAAGTCTGGAATGAATGTGGCTCGTCTGAACTTCTCTCA |
| as isoform M2, | TGGAACTCATGAGTACCATGCGGAGACCATCAAGAATGTGCGCACAGCCACGG |
| but contains a | AAAGCTTTGCTTCTGACCCCATCCTCTACCGGCCCGTTGCTGTGGCTCTAGACA |
| different internal | CTAAAGGACCTGAGATCCGAACTGGGCTCATCAAGGGCAGCGGCACTGCAGAG |
| segment. | GTGGAGCTGAAGAAGGGAGCCACTCTCAAAATCACGCTGGATAACGCCTACAT |
| | GGAAAAGTGTGACGAGAACATCCTGTGGCTGGACTACAAGAACATCTGCAAGG |
| | TGGTGGAAGTGGGCAGCAAGATCTACGTGGATGATGGGCTTATTTCTCTCCAG |
| | GTGAAGCAGAAAGGTGCCGACTTCCTGGTGACGGAGGTGGAAAATGGTGGCTC |
| | CTTGGGCAGCAAGAAGGGTGTGAACCTTCCTGGGGCTGCTGTGGACTTGCCTG |
| | CTGTGTCGGAGAAGGACATCCAGGATCTGAAGTTTGGGGTCGAGCAGGATGTT |
| | GATATGGTGTTTGCGTCATTCATCCGCAAGGCATCTGATGTCCATGAAGTTAGG |
| | AAGGTCCTGGGAGAGAAGGGAAAGAACATCAAGATTATCAGCAAAATCGAGA |
| | ATCATGAGGGGGTTCGGAGGTTTGATGAAATCCTGGAGGCCAGTGATGGGATC |
| | ATGGTGGCTCGTGGTGATCTAGGCATTGAGATTCCTGCAGAGAAGGTCTTCCTT |
| | GCTCAGAAGATGATGATTGGACGGTGCAACCGAGCTGGGAAGCCTGTCATCTG |
| | TGCTACTCAGATGCTGGAGAGCATGATCAAGAAGCCCCGCCCCACTCGGGCTG |
| | AAGGCAGTGATGTGGCCAATGCAGTCCTGGATGGAGCCGACTGCATCATGCTG |
| | TCTGGAGAAACAGCCAAAGGGGACTATCCTCTGGAGGCTGTGCGCATGCAGCA |
| | CCTGATAGCTCGTGAGGCTGAGGCAGCCATGTTCCACCGCAAGCTGTTTGAAG |
| | AACTTGTGCGAGCCTCAAGTCACTCCACAGACCTCATGGAAGCCATGGCCATG |
| | GGCAGCGTGGAGGCTTCTTATAAGTGTTTAGCAGCAGCTTTGATAGTTCTGACG |
| | GAGTCTGGCAGGTCTGCTCACCAGGTGGCCAGATACCGCCCACGTGCCCCCAT |
| | CATTGCTGTGACCCGGAATCCCCAGACAGCTCGTCAGGCCCACCTGTACCGTGG |
| | CATCTTCCCTGTGCTGTGCAAGGACCCAGTCCAGGAGGCCTGGGCTGAGGACG |
| | TGGACCTCCGGGTGAACTTTGCCATGAATGTTGGCAAGGCCCGAGGCTTCTTCA |
| | AGAAGGGAGATGTGGTCATTGTGCTGACCGGATGGCGCCCTGGCTCCGGCTTC |
| | ACCAACACCATGCGTGTTGTTCCTGTGCCGTGATGGACCCCAGAGCCCCTCCTC |
| | CAGCCCCTGTCCCACCCCCTTCCCCCAGCCCATCCATTAGGCCAGCAACGCTTG |
| | TAGAACTCACTCTGGGCTGTAACGTGGCACTGGTAGGTTGGGACACCAGGGAA |
| | GAAGATCAACGCCTCACTGAAACATGGCTGTGTTTGCAGCCTGCTCTAGTGGG |
| | ACAGCCCAGAGCCTGGCTGCCCATCATGTGGCCCCACCCAATCAAGGGAAGAA |
| | GGAGGAATGCTGGACTGGAGGCCCCTGGAGCCAGATGGCAAGAGGGTGACAG |
| | CTTCCTTTCCTGTGTGTACTCTGTCCAGTTCCTTTAGAAAAAATGGATGCCCAG |
| | AGGACTCCCAACCCTGGCTTGGGGTCAAGAAACAGCCAGCAAGAGTTAGGGGC |
| | CTTAGGGCACTGGGCTGTTGTTCCATTGAAGCCGACTCTGGCCCTGGCCCTTAC |
| | TTGCTTCTCTAGCTCTCTAGGCCTCTCCAGTTTGCACCTGTCCCCACCCTCCACT |
| | CAGCTGTCCTGCAGCAAACACTCCACCCTCCACCTTCCATTTTCCCCCACTACT |
| | GCAGCACCTCCAGGCCTGTTGCTATAGAGCCTACCTGTATGTCAATAAACAACA |
| | GCTGAAGCACC(SEQ ID NO: 51) |
| gi\|33286421\|ref\|N | CGCCGCGCTTCCTCCTGAAGGTGACTGCGCCCGCGGGGACGCAGGGGGCGGGG |
| NM_182471.1\| | CCCGGGTCGCCCGGAGCCGGGATTGGGCAGAGGGCGGGGCGGCGGAGGGATT |
| Homo sapiens | GCGGCGGCCCGCAGCGGGATAACCTTGAGGCTGAGGCAGTGGCTCCTTGCACA |
| pyruvate kinase, | GCAGCTGCACGCGCCGTGGCTCCGGATCTCTTCGTCTTTGCAGCGTAGCCCGAG |
| muscle (PKM2), | TCGGTCAGCGCCGGAGGACCTCAGCAGCCATGTCGAAGCCCCATAGTGAAGCC |
| transcript variant | GGGACTGCCTTCATTCAGACCCAGCAGCTGCACGCAGCCATGGCTGACACATT |
| 3, mRNA | CCTGGAGCACATGTGCCGCCTGGACATTGATTCACCACCCATCACAGCCCGGA |
| Transcript | ACACTGGCATCATCTGTACCATTGGCCCAGCTTCCCGATCAGTGGAGACGTTGA |
| Variant: This | AGGAGATGATTAAGTCTGGAATGAATGTGGCTCGTCTGAACTTCTCTCATGGAA |
| transcript variant | CTCATGAGTACCATGCGGAGACCATCAAGAATGTGCGCACAGCCACGGAAAGC |
| (3) lacks a | TTTGCTTCTGACCCCATCCTCTACCGGCCCGTTGCTGTGGCTCTAGACACTAAA |
| segment within | GGACCTGAGATCCGAACTGGGCTCATCAAGGGCAGCGGCACTGCAGAGGTGGA |
| the 5′ UTR, as | GCTGAAGAAGGGAGCCACTCTCAAAATCACGCTGGATAACGCCTACATGGAAA |
| compared to | AGTGTGACGAGAACATCCTGTGGCTGGACTACAAGAACATCTGCAAGGTGGTG |
| transcript variant | GAAGTGGGCAGCAAGATCTACGTGGATGATGGGCTTATTTCTCTCCAGGTGAA |
| 2. Both variants | GCAGAAAGGTGCCGACTTCCTGGTGACGGAGGTGGAAAATGGTGGCTCCTTGG |
| encode the same | GCAGCAAGAAGGGTGTGAACCTTCCTGGGGCTGCTGTGGACTTGCCTGCTGTGT |
| isoform (M1). | CGGAGAAGGACATCCAGGATCTGAAGTTTGGGGTCGAGCAGGATGTTGATATG |
| | GTGTTTGCGTCATTCATCCGCAAGGCATCTGATGTCCATGAAGTTAGGAAGGTC |
| | CTGGGAGAGAAGGGAAAGAACATCAAGATTATCAGCAAAATCGAGAATCATG |
| | AGGGGGTTCGGAGGTTTGATGAAATCCTGGAGGCCAGTGATGGGATCATGGTG |
| | GCTCGTGGTGATCTAGGCATTGAGATTCCTGCAGAGAAGGTCTTCCTTGCTCAG |
| | AAGATGATGATTGGACGGTGCAACCGAGCTGGGAAGCCTGTCATCTGTGCTAC |
| | TCAGATGCTGGAGAGCATGATCAAGAAGCCCCGCCCCACTCGGGCTGAAGGCA |
| | GTGATGTGGCCAATGCAGTCCTGGATGGAGCCGACTGCATCATGCTGTCTGGA |
| | GAAACAGCCAAAGGGGACTATCCTCTGGAGGCTGTGCGCATGCAGCACCTGAT |
| | AGCTCGTGAGGCTGAGGCAGCCATGTTCCACCGCAAGCTGTTTGAAGAACTTG |
| | TGCGAGCCTCAAGTCACTCCACAGACCTCATGGAAGCCATGGCCATGGGCAGC |
| | GTGGAGGCTTCTTATAAGTGTTTAGCAGCAGCTTTGATAGTTCTGACGGAGTCT |
| | GGCAGGTCTGCTCACCAGGTGGCCAGATACCGCCCACGTGCCCCCATCATTGCT |
| | GTGACCCGGAATCCCCAGACAGCTCGTCAGGCCCACCTGTACCGTGGCATCTTC |
| | CCTGTGCTGTGCAAGGACCCAGTCCAGGAGGCCTGGGCTGAGGACGTGGACCT |
| | CCGGGTGAACTTTGCCATGAATGTTGGCAAGGCCCGAGGCTTCTTCAAGAAGG |
| | GAGATGTGGTCATTGTGCTGACCGGATGGCGCCCTGGCTCCGGCTTCACCAACA |
| | CCATGCGTGTTGTTCCTGTGCCGTGATGGACCCCAGAGCCCCTCCTCCAGCCCC |
| | TGTCCCACCCCCTTCCCCCAGCCCATCCATTAGGCCAGCAACGCTTGTAGAACT |
| | CACTCTGGGCTGTAACGTGGCACTGGTAGGTTGGGACACCAGGGAAGAAGATC |
| | AACGCCTCACTGAAACATGGCTGTGTTTGCAGCCTGCTCTAGTGGGACAGCCCA |
| | GAGCCTGGCTGCCCATCATGTGGCCCCACCCAATCAAGGGAAGAAGGAGGAAT |
| | GCTGGACTGGAGGCCCCTGGAGCCAGATGGCAAGAGGGTGACAGCTTCCTTTC |
| | CTGTGTGTACTCTGTCCAGTTCCTTTAGAAAAAATGGATGCCCAGAGGACTCCC |
| | AACCCTGGCTTGGGGTCAAGAAACAGCCAGCAAGAGTTAGGGGCCTTAGGGCA |
| | CTGGGCTGTTGTTCCATTGAAGCCGACTCTGGCCCTGGCCCTTACTTGCTTCTCT |
| | AGCTCTCTAGGCCTCTCCAGTTTGCACCTGTCCCCACCCTCCACTCAGCTGTCCT |
| | GCAGCAAACACTCCACCCTCCACCTTCCATTTTCCCCCACTACTGCAGCACCTC |
| | CAGGCCTGTTGCTATAGAGCCTACCTGTATGTCAATAAACAACAGCTGAAGCA |
| | CCAAAAAAAAAAAAAAAAAAAAAAAAAAAAAAAAAAAAAAAA(SEQ ID NO: |
| | 52) |
| gi\|33286420\|ref\| | MSKPHSEAGTAFIQTQQLHAAMADTFLEHMCRLDIDSPPITARNTGIICTIGPASRSV |
| NP_872270.1\| | ETLKEMIKSGMNVARLNFSHGTHEYHAETIKNVRTATESFASDPILYRPVAVALDT |
| pyruvate kinase, | KGPEIRTGLIKGSGTAEVELKKGATLKITLDNAYMEKCDENILWLDYKNICKVVEV |
| muscle isoform | GSKIYVDDGLISLQVKQKGADFLVTEVENGGSLGSKKGVNLPGAAVDLPAVSEKD |
| M1 [Homo | IQDLKFGVEQDVDMVFASFIRKASDVHEVRKVLGEKGKNIKIISKIENHEGVRRFDE |
| sapiens] | ILEASDGIMVARGDLGIEIPAEKVFLAQKMMIGRCNRAGKPVICATQMLESMIKKP |
| | RPTRAEGSDVANAVLDGADCIMLSGETAKGDYPLEAVRMQHLIAREAEAAMFHR |
| | KLFEELVRASSHSTDLMEAMAMGSVEASYKCLAAALIVLTESGRSAHQVARYRPR |
| | APIIAVTRNPQTARQAHLYRGIFPVLCKDPVQEAWAEDVDLRVNFAMNVGKARGF |
| | FKKGDVVIVLTGWRPGSGFTNTMRVVPVP (SEQ ID NO: 53) |
| gi\|33286422\|ref\| | MSKPHSEAGTAFIQTQQLHAAMADTFLEHMCRLDIDSPPITARNTGIICTIGPASRSV |
| NP_872271.1\| | ETLKEMIKSGMNVARLNFSHGTHEYHAETIKNVRTATESFASDPILYRPVAVALDT |
| pyruvate kinase, | KGPEIRTGLIKGSGTAEVELKKGATLKITLDNAYMEKCDENILWLDYKNICKVVEV |
| muscle isoform | GSKIYVDDGLISLQVKQKGADFLVTEVENGGSLGSKKGVNLPGAAVDLPAVSEKD |
| M1 [Homo | IQDLKFGVEQDVDMVFASFIRKASDVHEVRKVLGEKGKNIKIISKIENHEGVRRFDE |
| sapiens] | ILEASDGIMVARGDLGIEIPAEKVFLAQKMMIGRCNRAGKPVICATQMLESMIKKP |
| | RPTRAEGSDVANAVLDGADCIMLSGETAKGDYPLEAVRMQHLIAREAEAAMFHR |
| | KLFEELVRASSHSTDLMEAMAMGSVEASYKCLAAALIVLTESGRSAHQVARYRPR |
| | APIIAVTRNPQTARQAHLYRGIFPVLCKDPVQEAWAEDVDLRVNFAMNVGKARGF |
| | FKKGDVVIVLTGWRPGSGFTNTMRVVPVP (SEQ ID NO: 54) |

TABLE 2
M2 Isoform-related sequences:

| TRANSCRIPT | |
|---|---|
| TYPE | SEQUENCE |
| gi\|33286417\|ref\| | CGCCGCGCTTCCTCCTGAAGGTGACTGCGCCCGCGGGGACGCAGGGGGCGGGG |
| NM_002654.3\| | CCCGGGTCGCCCGGAGCCGGGATTGGGCAGAGGGCGGGGCGGCGGAGGGATT |
| Homo sapiens | GCGGCGGCCCGCAGCGGGATAACCTTGAGGCTGAGGCAGTGGCTCCTTGCACA |
| pyruvate kinase, | GCAGCTGCACGCGCCGTGGCTCCGGATCTCTTCGTCTTTGCAGCGTAGCCCGAG |
| muscle (PKM2), | TCGGTCAGCGCCGGAGGACCTCAGCAGCCATGTCGAAGCCCCATAGTGAAGCC |
| transcript variant | GGGACTGCCTTCATTCAGACCCAGCAGCTGCACGCAGCCATGGCTGACACATT |
| 1, mRNA | CCTGGAGCACATGTGCCGCCTGGACATTGATTCACCACCCATCACAGCCCGGA |
| Transcript | ACACTGGCATCATCTGTACCATTGGCCCAGCTTCCCGATCAGTGGAGACGTTGA |
| Variant: This | AGGAGATGATTAAGTCTGGAATGAATGTGGCTCGTCTGAACTTCTCTCATGGAA |
| variant (1) lacks a | CTCATGAGTACCATGCGGAGACCATCAAGAATGTGCGCACAGCCACGGAAAGC |
| segment in the 5′ | TTTGCTTCTGACCCCATCCTCTACCGGCCCGTTGCTGTGGCTCTAGACACTAAA |
| UTR and has an | GGACCTGAGATCCGAACTGGGCTCATCAAGGGCAGCGGCACTGCAGAGGTGGA |
| alternate in-frame | GCTGAAGAAGGGAGCCACTCTCAAAATCACGCTGGATAACGCCTACATGGAAA |
| coding exon, as | AGTGTGACGAGAACATCCTGTGGCTGGACTACAAGAACATCTGCAAGGTGGTG |
| compared to | GAAGTGGGCAGCAAGATCTACGTGGATGATGGGCTTATTTCTCTCCAGGTGAA |
| variant 2. The | GCAGAAAGGTGCCGACTTCCTGGTGACGGAGGTGGAAAATGGTGGCTCCTTGG |
| resulting isoform | GCAGCAAGAAGGGTGTGAACCTTCCTGGGGCTGCTGTGGACTTGCCTGCTGTGT |
| (M2) has a | CGGAGAAGGACATCCAGGATCTGAAGTTTGGGGTCGAGCAGGATGTTGATATG |
| different internal | GTGTTTGCGTCATTCATCCGCAAGGCATCTGATGTCCATGAAGTTAGGAAGGTC |
| segment than | CTGGGAGAGAAGGGAAAGAACATCAAGATTATCAGCAAAATCGAGAATCATG |
| isoform M1, but | AGGGGGTTCGGAGGTTTGATGAAATCCTGGAGGCCAGTGATGGGATCATGGTG |
| is the same | GCTCGTGGTGATCTAGGCATTGAGATTCCTGCAGAGAAGGTCTTCCTTGCTCAG |
| length. | AAGATGATGATTGGACGGTGCAACCGAGCTGGGAAGCCTGTCATCTGTGCTAC |
| | TCAGATGCTGGAGAGCATGATCAAGAAGCCCCGCCCCACTCGGGCTGAAGGCA |
| | GTGATGTGGCCAATGCAGTCCTGGATGGAGCCGACTGCATCATGCTGTCTGGA |
| | GAAACAGCCAAAGGGGACTATCCTCTGGAGGCTGTGCGCATGCAGCACCTGAT |
| | TGCCCGTGAGGCAGAGGCTGCCATCTACCACTTGCAATTATTTGAGGAACTCCG |
| | CCGCCTGGCGCCCATTACCAGCGACCCCACAGAAGCCACCGCCGTGGGTGCCG |
| | TGGAGGCCTCCTTCAAGTGCTGCAGTGGGGCCATAATCGTCCTCACCAAGTCTG |
| | GCAGGTCTGCTCACCAGGTGGCCAGATACCGCCCACGTGCCCCCATCATTGCTG |
| | TGACCCGGAATCCCCAGACAGCTCGTCAGGCCCACCTGTACCGTGGCATCTTCC |
| | CTGTGCTGTGCAAGGACCCAGTCCAGGAGGCCTGGGCTGAGGACGTGGACCTC |
| | CGGGTGAACTTTGCCATGAATGTTGGCAAGGCCCGAGGCTTCTTCAAGAAGGG |
| | AGATGTGGTCATTGTGCTGACCGGATGGCGCCCTGGCTCCGGCTTCACCAACAC |
| | CATGCGTGTTGTTCCTGTGCCGTGATGGACCCCAGAGCCCCTCCTCCAGCCCCT |
| | GTCCCACCCCCTTCCCCCAGCCCATCCATTAGGCCAGCAACGCTTGTAGAACTC |
| | ACTCTGGGCTGTAACGTGGCACTGGTAGGTTGGGACACCAGGGAAGAAGATCA |
| | ACGCCTCACTGAAACATGGCTGTGTTTGCAGCCTGCTCTAGTGGGACAGCCCAG |
| | AGCCTGGCTGCCCATCATGTGGCCCCACCCAATCAAGGGAAGAAGGAGGAATG |
| | CTGGACTGGAGGCCCCTGGAGCCAGATGGCAAGAGGGTGACAGCTTCCTTTCC |
| | TGTGTGTACTCTGTCCAGTTCCTTTAGAAAAAATGGATGCCCAGAGGACTCCCA |
| | ACCCTGGCTTGGGGTCAAGAAACAGCCAGCAAGAGTTAGGGGCCTTAGGGCAC |
| | TGGGCTGTTGTTCCATTGAAGCCGACTCTGGCCCTGGCCCTTACTTGCTTCTCTA |
| | GCTCTCTAGGCCTCTCCAGTTTGCACCTGTCCCCACCCTCCACTCAGCTGTCCTG |
| | CAGCAAACACTCCACCCTCCACCTTCCATTTTCCCCCACTACTGCAGCACCTCC |
| | AGGCCTGTTGCTATAGAGCCTACCTGTATGTCAATAAACAACAGCTGAAGCAC |
| | CAAAAAAAAAAAAAAAAAAAAA (SEQ ID NO: 56) |
| gi\|33286418\|ref\| | MSKPHSEAGTAFIQTQQLHAAMADTFLEHMCRLDIDSPPITARNTGIICTIGPASRSV |
| NP_002645.3\| | ETLKEMIKSGMNVARLNFSHGTHEYHAETIKNVRTATESFASDPILYRPVAVALDT |
| pyruvate kinase, | KGPEIRTGLIKGSGTAEVELKKGATLKITLDNAYMEKCDENILWLDYKNICKVVEV |
| muscle isoform | GSKIYVDDGLISLQVKQKGADFLVTEVENGGSLGSKKGVNLPGAAVDLPAVSEKD |
| M2 [Homo | IQDLKFGVEQDVDMVFASFIRKASDVHEVRKVLGEKGKNIKIISKIENHEGVRRFDE |
| sapiens] | ILEASDGIMVARGDLGIEIPAEKVFLAQKMMIGRCNRAGKPVICATQMLESMIKKP |
| | RPTRAEGSDVANAVLDGADCIMLSGETAKGDYPLEAVRMQHLIAREAEAAIYHLQ |
| | LFEELRRLAPITSDPTEATAVGAVEASFKCCSGAIIVLTKSGRSAHQVARYRPRAPII |
| | AVTRNPQTARQAHLYRGIFPVLCKDPVQEAWAEDVDLRVNFAMNVGKARGFFKK |
| | GDVVIVLTGWRPGSGFTNTMRVVPVP (SEQ ID NO: 57) |

TABLE 3
R and L isoform-related sequences:

| TRANSCRIPT | |
|---|---|
| TYPE | SEQUENCE |
| gi\|10835121\|ref\| | MSIQENISSLQLRSWVSKSQRDLAKSILIGAPGGPAGYLRRASVAQLTQELGTAFFQ |
| NP_000289.1\| | QQQLPAAMADTFLEHLCLLDIDSEPVAARSTSIIATIGPASRSVERLKEMIKAGMNI |
| pyruvate kinase, | ARLNFSHGSHEYHAESIANVREAVESFAGSPLSYRPVAIALDTKGPEIRTGILQGGPE |
| liver and RBC | SEVELVKGSQVLVTVDPAFRTRGNANTVWVDYPNIVRVVPVGGRIYIDDGLISLVV |
| isoform 1 [Homo | QKIGPEGLVTQVENGGVLGSRKGVNLPGAQVDLPGLSEQDVRDLRFGVEHGVDIV |
| sapiens] | FASFVRKASDVAAVRAALGPEGHGIKIISKIENHEGVKRFDEILEVSDGIMVARGDL |
| | GIEIPAEKVFLAQKMMIGRCNLAGKPVVCATQMLESMITKPRPTRAETSDVANAVL |
| | DGADCIMLSGETAKGNFPVEAVKMQHAIAREAEAAVYHRQLFEELRRAAPLSRDP |
| | TEVTAIGAVEAAFKCCAAAIIVLTTTGRSAQLLSRYRPRAAVIAVTRSAQAARQVH |
| | LCRGVFPLLYREPPEAIWADDVDRRVQFGIESGKLRGFLRVGDLVIVVTGWRPGSG |
| | YTNIMRVLSIS (SEQ ID NO: 58) |
| gi\|32967597\|ref\| | MEGPAGYLRRASVAQLTQELGTAFFQQQQLPAAMADTFLEHLCLLDIDSEPVAAR |
| NP_870986.1\| | STSIIATIGPASRSVERLKEMIKAGMNIARLNFSHGSHEYHAESIANVREAVESFAGS |
| pyruvate kinase, | PLSYRPVAIALDTKGPEIRTGILQGGPESEVELVKGSQVLVTVDPAFRTRGNANTV |
| liver and RBC | WVDYPNIVRVVPVGGRIYIDDGLISLVVQKIGPEGLVTQVENGGVLGSRKGVNLPG |
| isoform 2 [Homo | AQVDLPGLSEQDVRDLRFGVEHGVDIVFASFVRKASDVAAVRAALGPEGHGIKIIS |
| sapiens] | KIENHEGVKRFDEILEVSDGIMVARGDLGIEIPAEKVFLAQKMMIGRCNLAGKPVV |
| | CATQMLESMITKPRPTRAETSDVANAVLDGADCIMLSGETAKGNFPVEAVKMQH |
| | AIAREAEAAVYHRQLFEELRRAAPLSRDPTEVTAIGAVEAAFKCCAAAIIVLTTTGR |
| | SAQLLSRYRPRAAVIAVTRSAQAARQVHLCRGVFPLLYREPPEAIWADDVDRRVQ |
| | FGIESGKLRGFLRVGDLVIVVTGWRPGSGYTNIMRVLSIS (SEQ ID NO: 59) |

As used herein, the term “exon 10 of pyruvate kinase” refers to the nucleotide sequence

(SEQ ID NO:50)
ATTGCCCGTGAGGCAGAGGCTGCCATCTACCACTTGCAATTATTTGAGGA
ACTCCGCCGCCTGGCGCCCATTACCAGCGACCCCACAGAAGCCACCGCCG
TGGGTGCCGTGGAGGCCTCCTTCAAGTGCTGCAGTGGGGCCATAATCGTC
CTCACCAAGT

Structure of PKM2-Inhibitory Nucleic Acid Molecules

In some embodiments, a PKM2-inhibitory nucleic acid molecule is provided that is an RNA molecule. In some embodiments, a PKM2-inhibitory nucleic acid molecule provided herein comprises a DNA molecule, or a DNA/RNA hybrid molecule. In some embodiments, a PKM2-inhibitory nucleic acid molecule is provided that comprises a modified nucleotide and/or a modified internucleotide linkage. In some embodiments, a double-stranded PKM2-inhibitory nucleic acid molecule is provided that is a siRNA molecule, comprising a sense and an antisense RNA strand. In some embodiments, a PKM2-inhibitory nucleic acid molecule provided is a shRNA, comprising a sense and an antisense strand connected by a linker sequence forming a loop, for example, a hairpin loop. In some embodiments, a PKM2-inhibitory nucleic acid molecule is a microRNA. In some embodiments, a PKM2-inhibitory nucleic acid molecule provided is an antisense RNA. In some embodiments, a PKM2-inhibitory nucleic acid provided is an expression construct comprising a promoter directing transcription of a primary transcript that can be processed by a target cell into a substrate for a member of the RNAi pathway, or a related pathway, for example a Dicer substrate.

In some embodiments, a nucleic acid molecule provided by aspects of this invention, comprises about 10 to about 50 contiguous nucleotides corresponding to a target sequence comprised, at least partially, in exon 10 of the human pyruvate kinase gene. In some embodiments, a nucleic acid molecule provided by aspects of this invention, comprises about 50 to about 100 contiguous nucleotides corresponding to a target sequence comprised, at least partially, in exon 10 of the human pyruvate kinase gene. In some embodiments, a nucleic acid molecule provided by aspects of this invention, comprises about 100 to about 160 contiguous nucleotides corresponding to a target sequence comprised, at least partially, in exon 10 of the human pyruvate kinase gene. In some embodiments, the nucleic acid molecule comprises at least about 19 to about 24 contiguous nucleotides corresponding to such a target sequence. In some embodiments, the nucleic acid molecule comprises at least about 25 to about 30 contiguous nucleotides corresponding to such a target sequence. In some embodiments, the nucleic acid molecule comprises at least about 30 to about 50 contiguous nucleotides corresponding to such a target sequence. In some embodiments, the nucleic acid molecule comprises at least about 50 to about 100 contiguous nucleotides corresponding to such a target sequence. In some embodiments, the nucleic acid molecule comprises at least about 100 to about 160 contiguous nucleotides corresponding to such a target sequence. In some embodiments, each strand of a double-stranded nucleic acid molecule provided by aspects of this invention comprises at least about 19 nucleotides that are complementary to the nucleotides of the other strand of the double-stranded nucleic acid molecule.

In some embodiments, a double-stranded siRNA molecule provided by the invention comprises a 3′ overhang of 1-4 nucleotides, and preferably of 2 nucleotides, on one or both strands. In some embodiments, the nucleotide overhangs are of the sequence 5′-UU-3′.

In some embodiments, a nucleic acid molecule provided by some aspects of this invention is a siRNA encoded by and expressed from a genomically integrated transgene or a plasmid-based expression vector. In some embodiments, the siRNA is encoded by an integrated transgene or a plasmid-based expression vector able to express a shRNA or microRNA. Methods of generating and expressing shRNA or microRNA constructs that yield functional siRNA molecules after processing by Dicer and other members of the RNAi machinery are well known in the art (see, e.g., Brummelkamp, T. R., Bernards, R. & Agami, R. A system for stable expression of short interfering RNAs in mammalian cells. Science 296, 550-553 (2002); Paddison, P. J., Caudy, A. A., Bernstein, E., Hannon, G. J. & Conklin, D. S. Short hairpin RNAs (shRNAs) induce sequence-specific silencing in mammalian cells. Genes Dev. 16, 948-958 (2002); Paul, C. P., Good, P. D., Winer, I. & Engelke, D. R. Effective expression of small interfering RNA in human cells. Nature Biotechnol. 20, 505-508 (2002); Miyagishi, M. & Taira, K. U6 promoter-driven siRNAs with four uridine 3′ overhangs efficiently suppress targeted gene expression in mammalian cells. Nature Biotechnol. 20, 497-500 (2002); Lee, N. S. et al. Expression of small interfering RNAs targeted against HIV-1 rev transcripts in human cells. Nature Biotechnol. 20, 500-505 (2002); Paddison, P. J. & Hannon, G. J. RNA interference: the new somatic cell genetics? Cancer Cell 2, 17-23 (2002); Brummelkamp, T. R., Bernards, R. & Agami, R. Stable suppression of tumorigenicity by virus-mediated RNA interference. Cancer Cell 2, 243-247 (2002); Dirac, A. M. & Bernards, R. Reversal of senescence in mouse fibroblasts through lentiviral suppression of p53. J. Biol. Chem. 278, 11731-11734 (2003); Abbas-Terki, T., Blanco-Bose, W., Deglon, N., Pralong, W. & Aebischer, P. Lentiviral-mediated RNA interference. Hum. Gene Ther. 13, 2197-2201 (2002); Rubinson, D. A. et al. A lentivirus-based system to functionally silence genes in primary mammalian cells, stem cells and transgenic mice by RNA interference. Nature Genet. 33, 401-406 (2003); Scherr, M., Battmer, K., Ganser, A. & Eder, M. Modulation of gene expression by lentiviral-mediated delivery of small interfering RNA. Cell Cycle 2, 251-257 (2003); and Qin, X. F., An, D. S., Chen, I. S. & Baltimore, D. Inhibiting HIV-1 infection in human T cells by lentiviral-mediated delivery of small interfering RNA against CCR5. Proc. Natl. Acad. Sci. USA 100, 183-188 (2003); all of which are incorporated in their entirety herein by reference for methods of generating and expressing shRNA or microRNA constructs).

Both RNA-polymerase II and RNA-polymerase III promoters have been reported to efficiently drive expression of shRNAs and microRNAs and expression constructs driving expression from either type of promoter are within the scope of some embodiments of the current invention. The use of a RNA-polymerase II promoter allows the application of a wide array of regulatory elements well known in the art of protein expression, including inducible and tissue-specific expression systems. In some embodiments, transgenes and expression vectors are controlled by tissue specific promoters, for example tumor-specific promoters. In other embodiments transgenes and expression vectors are controlled by inducible promoters, such as tetracycline inducible expression systems.

As used herein, the term “sense strand” refers to the strand of a nucleic acid molecule that is complementary to a given nucleotide sequence, for example a DNA sequence that can be transcribed into RNA. In natural transcription, the RNA strand transcribed from the sense DNA strand is a sense RNA strand and has the same nucleotide sequence as the sense DNA strand, except that it contains ribonucleotides instead of deoxyribonucleotides. An antisense strand is a nucleic acid molecule strand that is complementary to a sense strand. Accordingly, the sense strand of the PKM2 DNA includes the siRNA target sequence, for example, any of the exemplary target sequences given in SEQ ID NOs 1-14. For example, the sequence given in SEQ ID NO: 13, 5′-GCCATAATCGTCCTCACCA-3′, is the PKM2 exon 10 target sequence to which si155 corresponds. The complementary antisense siRNA strand to this target sequence is given in SEQ ID NO: 43 (5′-UGGUGAGGACGAUUAUGGC-3′) and the complementary siRNA sense strand is 5′-GCCAUAAUCGUCCUCACCA-3′ (SEQ ID NO: 60).

As used herein, the term “a nucleotide sequence corresponding to a target sequence” refers to a nucleotide sequence that is at least highly similar, but not necessarily identical to, a given target sequence, or the antisense sequence of the target sequence. Highly similar sequences in this context are sequences of nucleic acid molecules that can hybridize under physiological conditions to a nucleic acid comprising the target sequence or the antisense sequence of the target sequence. Accordingly, the term includes nucleotide sequences that are identical to the sense or the antisense strand of a given target sequence, and sequences that are highly similar, for example sequences that share about 99%, about 98%, about 97%, about 96%, about 95%, about 94%, about 93%, about 92%, about 91%, about 90%, about 89%, about 88%, about 87%, about 86%, about 85%, about 84%, about 83%, about 82%, about 81, or about 80% sequence identity with the target sequence. Both the sense and the antisense strand of a given siRNA targeting PK mRNA, would, accordingly, correspond to their sense target sequence on the PK mRNA, even though none of them might be identical to the target sequence.

As used herein, the terms “decreasing expression” and “inhibiting expression” refer to decreasing expression at a transcriptional, post-transcriptional, and post-translational level or any combination thereof. While not wishing to be bound by theory, it is believed that the nucleic acid molecules provided herein regulate the expression of genes related to their target sequences on a post-transcriptional level, either by destabilizing gene transcripts or by inhibiting translation of transcripts via RNA interference mechanisms. However, other mechanisms of decreasing expression effected by nucleic acid molecules provided herein are expressly included in the scope of some aspects of this invention.

As used herein, the term “substantially inhibiting expression of a gene” refers to an inhibition of gene expression at a level of biological significance. For example, a nucleic acid that substantially inhibits expression of a gene is one that causes a detectable phenotypic change. In some embodiments, a detectable phenotypic change is a detectable change in cell viability, cell proliferation rate, cell shape, cell size, cell motility, and/or cell mobility. Similarly, a nucleic acid as provided herein that “does not substantially inhibit the expression of isoform M1 of pyruvate kinase (PKM1)” may not affect the expression of PKM1 at all, but may also inhibit the expression of PKM1 as long as the inhibition does not result in a biologically significant alteration of the affected biological system. A biological system is any living organism or cellular portion thereof, such as a cell, organ, tissue or the intact organism. Examples of a biologically significant alteration of a target cell would be a change in cell viability, cell mortality, cell metabolism, or cell differentiation state, such as tumor proliferation. Examples of a biologically significant alteration of a subject would be a significant change in life expectancy or in any function of any body part or organ of the subject. In some embodiments, a nucleic acid molecule that “does not substantially inhibit the function of an isoform M1” does not decrease wild type PKM1 protein levels by more than about 5%, more than about 10%, more than about 20%, more than about 25%, more than about 30%, more than about 40%, or more than about 50%. In some embodiments, a nucleic acid molecule that “does not substantially inhibit the function of an isoform M1” does not decrease wild type PKM1 protein levels to an extent that it limits a vital function of normal cells.

In some embodiments, a siRNA is provided that substantially inhibits expression of PKM2, but does not substantially inhibit expression of PKM1 in a cell. Such inhibition of one isoform without substantial inhibition of the other isoform is also referred to as “isoform-specific knockdown” or “specific knockdown” herein. In some embodiments, a siRNA is provided targeting a sequence that is comprised, at least partially, in exon 10 of the human pyruvate kinase gene, and that comprises at least 1, at least 2, at least 3, at least 4, at least 5, at least 6, at least 7, at least 8, at least 9, at least 10, least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, or at least 39 nucleotide mismatches as compared to the corresponding sequence of exon 9 of the human pyruvate kinase gene. For example, sit, as described in Table 4, targets a sequence comprising 1 nucleotide mismatch, si27, as described herein, targets a sequence that comprises 4 nucleotide mismatches, si155, as described herein, targets a sequence that comprises 8 nucleotide mismatches, and si156, as described herein, targets a sequence that comprises 8 nucleotide mismatches as compared to the corresponding sequence of exon 9.

As used herein, the term “small interfering RNA” (“siRNA”), also sometimes referred to in the art as “short interfering RNA” or “silencing RNA”, refers to a class of double-stranded RNA molecules that are preferably about 10-40 nucleotides in length, and preferably about 19-25 nucleotides in length, and are able to mediate RNA interference (RNAi). In preferred embodiments, an siRNA provided herein interferes with the expression of a specific gene, for example PKM2.

In general, siRNAs comprise an “RNA duplex stem”, referring to a double stranded structure of two annealed complementary RNA strands (sense and antisense). In some embodiments, sense and antisense strand are not 100% complementary and the duplex may comprise 1, 2, 3, 4, 5, 6, 7, 8, 9, or 10 mismatched nucleotides. Further, siRNAs optionally comprise 3′-overhangs of 2 nucleotides in length on either strand. Generally, each strand in an siRNA comprises a 5′ phosphate group and a 3′ hydroxyl (—OH) group. However, siRNAs with strand end modifications are also included in the scope of this invention.

An siRNA can be produced synthetically by methods well known to those in the art. In general, the sense and antisense strand of a synthetic siRNA are synthesized separately and annealed under conditions well known in the art to form the complete, double-stranded siRNA. An siRNA can also be produced within a cell by expressing a small hairpin RNA (shRNA) as a single transcript comprising a sense strand, a hairpin loop, and an antisense strand. Without wishing to be bound by theory, such a transcript is thought to form an RNA duplex in which sense and antisense strand are connected by the hairpin loop. Such small hairpin RNAs are believed to be processed by Dicer, an enzyme converting small hairpin RNAs and long double-stranded RNAs into siRNAs of the structure described above. Synthetic siRNAs, endogenous or exogenous shRNAs, as well as any artificially expressed double-stranded RNA or RNA derivative specifically targeting PKM2 are within the scope of this invention.

Without wishing to be bound by theory, it is believed that a key cellular pathway that uses small RNAs, such as siRNAs as provided herein, as sequence-specific regulators is the RNA interference (RNAi) pathway. RNAi has been proposed as a cellular response to the presence of double-stranded RNA (dsRNA) in the cell. The dsRNAs are cleaved into ˜20-base pair (bp) duplexes of small-interfering RNAs (siRNAs) by Dicer. It is further believed that siRNAs, either exogenously introduced into a cell or generated by Dicer from shRNA, microRNA, or other substrates bind to the RNA-induces silencing complex (“RISC”), where they are unwound and the sense strand, also called the “passenger strand” is discarded. The antisense strand of the siRNA, also referred to as the “guide strand”, complexed with RISC then binds to a complementary target sequence, for example, a target sequence comprised in an mRNA, which is subsequently cleaved by Slicer, resulting in inactivation of the nucleic acid molecule comprising the target sequence. As a result, the expression of mRNAs containing the target sequence is reduced. Target sequence recognition is, however, not completely stringent, as it is known in the art that the expression of mRNAs containing sequences highly similar to the target sequence are also sometimes affected by some siRNAs. The down-regulation of expression of mRNAs containing sequences that are similar but not identical to the target sequence of a given siRNA are refereed to as “off-target effects” and much effort has been undertaken in minimizing off-target effects of siRNA technology. These off-target effects can become problematic in cases where the mRNA targeted for downregulation is highly similar to another mRNA the downregulation of which is undesirable. It is believed that some aspects of biogenesis, protein complexes, and function are shared between the siRNA pathway and the miRNA pathway. In some embodiments of the instant invention, a nucleic acid molecule is provided that mimics the dsRNA in the siRNA mechanism, or the microRNA in the miRNA mechanism. siRNAs that can distinguish between splice variants M1 and M2 without producing off target effects have been discovered herein. The discovery of siRNAs having these properties was quite unexpected.

In some embodiments, a nucleic acid molecule is provided that is capable of associating with Dicer or a RISC complex. In some embodiments, a nucleic acid molecule is provided that is not a substrate for Dicer.

As used herein, the term “target sequence” refers to a nucleotide sequence targeted by a nucleic acid molecule, for example a nucleic acid molecule provided herein, as part of the RNAi mechanism. For example, a PKM2 target sequence would be a sequence of the mRNA of PKM2 which a given nucleic acid molecule, for example a nucleic acid molecule complexed with RISC would bind. In some embodiments, a target sequence is a sequence that is specific for the transcript to be targeted. For example, in some embodiments, a PKM2 target sequence is a sequence unique to the PKM2 mRNA.

As used herein, the term “nucleic acid molecule”, refers to a polymer of nucleotides. The term includes, but is not limited to, oligonucleotides and polynucleotides, and single-stranded and double-stranded forms, including hybrids, for example of DNA and RNA strands, or of strands comprising ribonucleotides, deoxyribonucleotides, and/or modified nucleotides. The polymer may include natural nucleosides (i.e., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyl adenosine, 5-methylcytidine, C5-bromouridine, C5-fluorouridine, C5-iodouridine, C5-propynyl-uridine, C5-propynyl-cytidine, C5-methylcytidine, 7-deazaadenosine, 7-deazaguanosine, 8-oxoadenosine, 8-oxoguanosine, O(6)-methylguanine, 4-acetylcytidine, 5-(carboxyhydroxymethyl)uridine, dihydrouridine, methylpseudouridine, 1-methyl adenosine, 1-methyl guanosine, N6-methyl adenosine, and 2-thiocytidine), chemically modified bases, biologically modified bases (e.g., methylated bases), intercalated bases, modified sugars (e.g., 2′-fluororibose, ribose, 2′-deoxyribose, 2′-O-methylcytidine, arabinose, and hexose), or modified phosphate groups (e.g., phosphorothioates and 5′-N-phosphoramidite linkages).

As used herein, the term “PKM2-inhibitory nucleic acid molecule” refers to a nucleic acid molecule that is able to inhibit PKM2 expression. In a clinical context, such a molecule is also sometimes referred to as a “therapeutic nucleic acid molecule”. In preferred embodiments a “PKM2-inhibitory nucleic acid molecule” is specific for PKM2 in that it does not inhibit expression of PKM1.

As used herein, the term “nucleotide” refers to a molecule comprising a nucleoside and one to three phosphate groups. The term “nucleoside” refers to a nucleobase covalently bound to a sugar moiety, for example, a ribose or deoxyribose. Naturally occurring nucleosides comprise one of the nucleobases adenine, thymine, guanine, cytosine, uracil and a ribose or a deoxyribose. As used herein, the term nucleotide also includes modified nucleotides, for example nucleotides comprising non-natural nucleosides (e.g., comprising a base or a sugar not naturally found in a nucleoside) and/or modified phosphate groups, for example a phosphorothioate group or other reactive group for internucleotide linkage. Nucleosides include nucleobases linked to amino acids or amino acid analogs which may comprise free carboxyl groups, free amino groups, or protecting groups. Suitable protecting groups are well known in the art (see P. G. M. Wuts and T. W. Greene, “Protective Groups in Organic Synthesis”, 2nd Ed., Wiley-Interscience, New York, 1999).

As used herein, the term “modified linkage” includes any analog or derivative of a phosphodiester linkage group that covalently couples adjacent nucleomonomers. Modified linkages include phosphodiester analogs, e.g., phosphorothioate, phosphorodithioate, and P-ethyoxyphosphodiester, P-ethoxyphosphodiester, P-alkyloxyphosphotriester, methylphosphonate, and nonphosphorus containing linkages, e.g., acetals and amides. Modified internucleotide linkages are known in the art (e.g., Bjergarde et al. 1991. Nucleic Acids Res. 19:5843; Caruthers et al. 1991. Nucleosides Nucleotides. 10:47). In certain embodiments, non-hydrolizable linkages are preferred, such as phosphorothioate linkages.

### PKM2-Inhibitory Nucleic Acid Molecule Modifications

PKM2-inhibitory nucleic acid molecules as provided by some aspects of the invention can be unmodified or modified, chemically synthesized, expressed from a vector or enzymatically synthesized. For example, in some embodiments that involve the expression of a nucleic acid molecule provided by this invention from an expression construct in a target cell, the nucleic acid molecule will generally be unmodified. In some embodiments, for example, embodiments providing a synthetic nucleic acid molecule, the nucleic acid molecule provided may comprise at least one modification. In some embodiments, synthetic, chemically-modified PKM2-inhibitory nucleic acid molecules are provided that are characterized by increased resistance to nuclease degradation in vivo, by improved cellular uptake, and/or by improved RNAi activity compared to that of unmodified nucleic acid molecules.

As used herein, the term “oligonucleotide” refers to a polymer of nucleotides that is between about 5 and about 250 nucleotides in length.

As used herein, the terms “linkage” and “internucleotide linkage” refers a naturally occurring, unmodified phosphodiester moiety (—O—(PO²⁻)—O—) that covalently couples adjacent nucleomonomers.

Some aspects of this invention provide PKM2-inhibitory nucleic acid molecules with a modification of a base, a sugar and/or a phosphate group that inhibits or prevents degradation by nucleases and/or increases potency in inhibiting expression of PKM2. Such modifications are well known to those of skill in the art (see e.g., Eckstein et al., International Publication No. WO 92/07065; Perrault et al, 1990 Nature 344, 565; Pieken et al., 1991, Science 253, 314; Usman and Cedergren, 1992, Trends in Biochem. Sci. 17, 334; Usman et al., International Publication No. WO 93/15187; and Rossi et al., International Publication No. WO 91/03162; Sproat, U.S. Pat. No. 5,334,711; and Burgin et al., Biochemistry 1996 Nov. 12; 35(45):14090-7; all of which are incorporated by reference herein).

Nucleic acid molecule modifications that result in enhanced stability and/or enhanced biological potency are known in the art and include, for example, 2′ amino, 2′-C-allyl, 2′-fluoro, 2′-O-methyl, 2′-H, nucleotide base modifications (for a review see Usman and Cedergren, 1992, TIBS. 17, 34; Usman et al., 1994, Nucleic Acids Symp. Ser. 31, 163; Burgin et al., 1996, Biochemistry, 35, 14090, all of which are incorporated by reference herein). Advantageous sugar modification of nucleic acid molecules have been described in the art (see, e.g., Eckstein et al., International Publication PCT No. WO 92/07065; Perrault et al. Nature, 1990, 344, 565 568; Pieken et al. Science, 1991, 253, 314317; Usman and Cedergren, Trends in Biochem. Sci., 1992, 17, 334 339; Usman et al. International Publication PCT No. WO 93/15187; Sproat, U.S. Pat. No. 5,334,711 and Beigelman et al., 1995, J. Biol. Chem., 270, 25702; Beigelman et al., International PCT publication No. WO 97/26270; Beigelman et al., U.S. Pat. No. 5,716,824, all of which are incorporated herein by reference).

Modified nucleic acid molecules as provided herein comprise, in some embodiments, a modified sugar moiety, including, for example, replacement of one or more of the hydroxyl groups with a halogen, a heteroatom, an aliphatic group, or the functionalization of the hydroxyl group as an ether, an amine, a thiol, or the like.

Administration of nucleic acid molecules, for example siRNA molecules as provided herein, are known to elicit cellular stress responses, for example, an interferon response, under certain circumstances, and modifications of inhibitory nucleic acid molecules decreasing or eliminating an undesired interferon response are known in the art. Without wishing to be bound by theory, it is believed that nucleic acid molecules as provided by aspects of this invention, for example, siRNA molecules, comprising a 2′-modification at the sugar moiety are not recognized by cellular machinery that is thought to recognize unmodified nucleic acid molecules. The use of 2′-modified or partially 2′-modified nucleic acid molecules, for example PKM2-inhibitory siRNA molecules, can decrease a cellular stress response, for example, an interferon response, to double-stranded nucleic acid molecules, while maintaining RNAi-mediated inhibition of the expression of a protein target, for example PKM2.

In some embodiments, a PKM2-inhibitory nucleic acid molecule is provided in which one, some or all pyrimidine nucleotides comprise a 2′-O-alkyl modification, for example a 2′-O-methyl modification or a 2′-O-allyl modification. In some embodiments, a nucleic acid molecule is provided comprising a 2′-modified ribose sugar, such as 2′-O-methyl modified nucleotide, for example at position 1, 2, 3, 4, 5, or 6, from the 5′-end of the nucleic acid molecule. In some embodiments, each strand of a provided double-stranded PKM2-inhibitory nucleic acid molecule comprises a single ribose modification and no other nucleotide modifications. In some embodiments, the modification comprises 2′-O-methyl modifications at alternative nucleotides, starting from either the first or the second nucleotide from the 5′-end.

Other sugar modifications comprised by nucleic acid molecules, for example, double-stranded PKM2-inhibitory siRNA molecules, provided herein include, for example, D-ribose, 2′-O-alkyl (including 2′-O-methyl and 2′-O-ethyl), i.e., 2′-alkoxy, 2′-amino, 2′-S-alkyl, 2′-halo (including 2′-fluoro), 2′-methoxyethoxy, 2′-allyloxy (—OCH₂CH═CH₂), 2′-propargyl, 2′-propyl, ethynyl, ethenyl, propenyl, and cyano. In some embodiments, PKM2-inhibitory nucleic acid molecules are provided comprising a nucleotide in which the sugar moiety is a hexose (Augustyns, K., et al., Nucl. Acids. Res. 18:4711 (1992)). Exemplary nucleomonomers can be found, e.g., in U.S. Pat. No. 5,849,902, incorporated by reference herein.

In some embodiments, a PKM2-inhibitory nucleic acid molecule is provided that comprises a modified internucleotide linkage group, for example, a phosphate analog linkage group, or a phosphorothioate linkage group. In some embodiments, nucleotides on both strands of a double-stranded nucleic acid molecule comprise a modified internucleotide linkage group. In some embodiments, modified internucleotide linkage is limited to 1, 2, 3, 4, 5, 6 or more nucleotides within the guide strand of the nucleic acid molecule. In some embodiments, 1, 2, 3, 4, 5, 6 or more nucleotide(s) of the guide strand within the double-stranded region of a PKM2-inhibitory nucleic acid molecule provided comprise a modified linkage group. In certain embodiments, the total number of nucleotides within a double-stranded nucleic acid molecule provided herein that comprise a modified linkage group, for example a phosphorothioate linkage group, is about 12-14. In some embodiments, the nucleotides comprising a modified internucleotide linkage group are not contiguous.

In certain embodiments, a double-stranded nucleic acid molecule able to inhibit PKM2 expression is provided that comprises one or more universal base-pairing nucleotides. Universal base-pairing nucleotides include extendable nucleotides that can be incorporated into a polynucleotide strand (either by chemical synthesis or by a polymerase), and pair with more than one pairing type of specific canonical nucleotide. Universal base pairing nucleotides are known in the art, see, for example, Berger et al., “Universal bases for hybridization, replication and chain termination,” Nucleic Acids Research 28(15): 2911-2914, 2000; Loakes et al., “Survey and Summary: The applications of universal DNA base analogues,” Nucleic Acid Research 29(12): 2437-2447, 2001; Nichols et al., “A universal nucleoside for use at ambiguous sites in DNA primers,” Nature 369:492-493, 1994; Fasman, 1989, Practical Handbook of Biochemistry and Molecular Biology, pp. 385 394, CRC Press, Boca Raton, Fla., and the references cited therein; U.S. Pat. No. 7,169,557 (all incorporated herein by reference).

Exemplary universal nucleotides include, but are not limited to, inosine-based nucleotide, 2′-deoxy-7-azaindole-5′-triphosphate (d7AITP), 2′-deoxy-isocarbostyril-5′-triphosphate (dICSTP), 2′-deoxy-propynylisocarbostyril-5′-triphosphate (dPICSTP), 2′-deoxy-6-methyl-7-azaindole-5′-triphosphate (dM7AITP), 2′-deoxy-imidizopyridine-5′-triphosphate (dlmPyTp), 2′-deoxy-pyrrollpyrizine-5′-triphosphate (dPPTP), 2′-deoxy-propynyl-7-azaindole-5′-triphosphate (dP7AITP), and 2′-deoxy-allenyl-7-azaindole-5′-triphosphate (dA7AITP).

Some modified nucleic acid molecules provided by aspects of this invention comprise nucleomonomers that contain a non-naturally occurring base (instead of a naturally occurring base), such as uridines or cytidines modified at the 5′-position, e.g., 5′-(2-amino)propyl uridine and 5′-bromo uridine; adenosines and guanosines modified at the 8-position, e.g., 8-bromo guanosine; deaza nucleotides, e.g., 7-deaza-adenosine; and N-alkylated nucleotides, e.g., N6-methyl adenosine.

In some embodiments, nucleic acid molecules provided herein comprise a 3′ and/or a 5′ terminus that is substantially protected from nucleases e.g., by modifying the 3′ or 5′ linkages (see, e.g., U.S. Pat. No. 5,849,902 and WO 98/13526), for example, by inclusion of a “blocking group”. In some embodiments, a nucleic acid molecule is provided, comprising a substituent (e.g., other than a hydroxy or phosphate group), for example, as a protecting group or coupling group for synthesis (e.g., FITC, propyl (CH₂—CH₂—CH₃), glycol (—O—CH₂—CH₂—O—) phosphate (PO₃²⁻), hydrogen phosphonate, or phosphoramidite). In some embodiments, a nucleic acid molecule is provided that comprises a blocking group which protects the 5′ and/or the 3′ terminus. End-blocking groups are known in the art and include, for example, cap structures (e.g., a 7-methylguanosine cap), inverted nucleomonomers, e.g., with 3′-3′ or 5′-5′ end inversions (see, e.g., Ortiagao et al. 1992. Antisense Res. Dev. 2:129), methylphosphonate, phosphoramidite, non-nucleotide groups (e.g., non-nucleotide linkers, amino linkers, conjugates). In some embodiments, a PKM2-inhibitory nucleic acid molecule is provided in which the 3′ terminal nucleomonomer comprises a modified sugar moiety, for example, a modified sugar moiety, in which the 3′-O is substituted by a blocking group that prevents 3′-exonuclease degradation of the oligonucleotide. For example, the 3′-hydroxyl can be esterified to a nucleotide through a 3′→3′ internucleotide linkage. Optionally, the 3′→3′ linked nucleotide at the 3′ terminus can be linked by a substitute linkage. To reduce nuclease degradation, the 5′ most 3′→5′ linkage can be a modified linkage, e.g., a phosphorothioate or a P-alkyloxyphosphotriester linkage. Preferably, the two 5′ most 3′→5′ linkages are modified linkages. Optionally, the 5′ terminal hydroxy moiety can be esterified with a phosphorus containing moiety, e.g., phosphate, phosphorothioate, or P-ethoxyphosphate.

It will be understood by those of skill in the art that the modifications described in detail herein are only exemplary in nature and that the invention includes molecules comprising any advantageous modification of nucleic acid molecules able to inhibit target gene expression known to those of skill in the art. Further, various nucleotide modifications may be combined.

### Synthesis of PKM2-Inhibitory Nucleic Acid Molecules

PKM2-inhibitory nucleic acid molecules as provided by some embodiments of the invention can be synthesized by any method known in the art, e.g., using enzymatic synthesis and/or chemical synthesis. In general, chemical synthesis can be used for the synthesis of unmodified or modified nucleic acid molecules provided herein. Chemical synthesis of linear nucleic acid molecules is well known in the art and can be achieved by solution or solid phase techniques. Nucleic acid molecules provided herein can be generated by any of several different synthetic procedures known in the art, including, but not limited to, the phosphoramidite, phosphite triester, H-phosphonate, and phosphotriester methods, typically by automated synthesis methods.

Nucleic acid molecule synthesis protocols are well known in the art and can be found, e.g., in U.S. Pat. No. 5,830,653; WO 98/13526; Stec et al. 1984. J. Am. Chem. Soc. 106:6077; Stec et al. 1985. J. Org. Chem. 50:3908; Stec et al. J. Chromatog. 1985. 326:263; LaPlanche et al. 1986. Nucl. Acid. Res. 1986. 14:9081; Fasman G. D., 1989. Practical Handbook of Biochemistry and Molecular Biology. 1989. CRC Press, Boca Raton, Fla.; Lamone. 1993. Biochem. Soc. Trans. 21:1; U.S. Pat. No. 5,013,830; U.S. Pat. No. 5,214,135; U.S. Pat. No. 5,525,719; Kawasaki et al. 1993. J. Med. Chem. 36:831; WO 92/03568; U.S. Pat. No. 5,276,019; and U.S. Pat. No. 5,264,423.

The synthesis method selected can depend on the length of the nucleic acid molecule to be produced. For example, the phosphoramidite and phosphite triester method can produce nucleic acid molecules having 175 or more nucleotides, while the H-phosphonate method works well for nucleic acid molecules of less than 100 nucleotides. Synthesis methods for the generation of modified nucleic acid molecules, as provided in some embodiments of this invention, are known in the art. For example, Uhlmann et al. (1990, Chemical Reviews 90:543-584) provide references and outline procedures for making oligonucleotides with modified bases and modified phosphodiester linkages. Other exemplary methods for making oligonucleotides are taught in Sonveaux. 1994. “Protecting Groups in Oligonucleotide Synthesis”; Agrawal. Methods in Molecular Biology 26:1. Exemplary synthesis methods are also taught in “Oligonucleotide Synthesis—A Practical Approach” (Gait, M. J. IRL Press at Oxford University Press. 1984).

Other exemplary synthesis techniques are well known in the art c; DNA Cloning, Volumes I and II (D N Glover Ed. 1985); Oligonucleotide Synthesis (M J Gait Ed, 1984; Nucleic Acid Hybridisation (B D Hames and S J Higgins eds. 1984); A Practical Guide to Molecular Cloning (1984); or the series, Methods in Enzymology (Academic Press, Inc.)).

Moreover, linear nucleic acid molecules of defined sequence, including nucleic acid molecules provided by some embodiments of this invention, and modified nucleic acid molecules, are readily available from commercial sources.

Generally, nucleic acid molecules are synthesized as single-stranded molecules. In some embodiments, antisense and sense strand of a PKM2-inhibitory nucleic acid molecule as provided herein are synthesized separately and combined to form a double-stranded nucleic acid molecule. Suitable conditions and methods for generating double-stranded nucleic acid molecules from separately synthesized strands are well known to those in the art.

In some embodiments, a nucleic acid molecule able to inhibit PKM2 expression is provided that is transcribed from an expression vector. Methods and reagents for the expression of the nucleic acid molecules provided herein in a target cell or in vitro are well known to those of skill in the art. The transcribed nucleic acid molecules may be isolated and purified, before desired modifications (such as replacing an unmodified sense strand with a modified one, etc.) are carried out.

In some embodiments, a nucleic acid molecule provided by the invention is expressed in a mammalian target cell, for example, a human cancer cell, using a mammalian expression vector. The mammalian expression vector may be capable of directing expression of the nucleic acid molecule, for example, in a particular cell type (e.g., tissue-specific regulatory elements are used to express the nucleic acid). Constitutive, ubiquitously expressed promoters and conditional, for example tissue-specific or drug-inducible promoters and regulatory elements are known in the art. Non-limiting examples of suitable promoters include CAGGS promoter, CMV promoter, ubiquitin promoter, H1 promoter, U6 promoter, tetracycline-inducible promoters, tissue-specific promoters, such as myosin heavy chain promoter, albumin promoter, lymphoid-specific promoters, neuron specific promoters, pancreas specific promoters, and mammary gland specific promoters. Developmentally-regulated promoters are also encompassed, for example the murine hox promoters and the alpha-fetoprotein promoter.

### Methods for Inhibiting PKM2 Expression in Target Cells

Some aspects of the invention provide a method for inhibiting the expression of PKM2 in a target cell, comprising contacting the target cell with a PKM2 inhibitory nucleic acid molecule provided by some aspects of this invention. The method may be carried out in vitro, ex vivo, or in vivo. The target cell (e.g., a human cancer cell) may be contacted with a PKM2 inhibitory nucleic acid molecule as provided herein in the presence of a delivery reagent, for example, a transfection reagent, such as a lipid (e.g., a cationic lipid) or a liposome. Suitable delivery reagents, for example, transfection reagents, as well as methods of formulating nucleic acid molecules with such delivery reagents are well known in the art.

Some aspects of the invention provide a method for inhibiting the expression of PKM2 in a target cell, comprising contacting the target cell with a vector expressing a PKM2 inhibitory nucleic acid.

As used herein, the term “contacting a cell” with a nucleic acid molecule provided by aspects of this invention described herein refers to any method of placing a cell in contact with such a nucleic acid. In preferred embodiments, a nucleic acid provided herein is shuttled into the cell either during or as a result of the contacting. In some embodiments, the nucleic acid molecule provided herein is accessible to dicer and the Risc complex as a result of the contacting. In some embodiments, contacting may comprise bringing a target cell into contact with a nucleic acid molecule provided herein under conditions suitable for cellular uptake of the nucleic acid molecule by the cell. In some embodiments, the nucleic acid is complexed with a transfection reagent, for example a lipid cation, a dendrimer, a polymer, or a nanoparticle to facilitate cellular uptake of the nucleic acid. Various methods of contacting a cell with a nucleic acid provided herein are well known to those of skill in the related arts. Such methods may comprise the use of a transfection reagent, for example a commercially available transfection reagent, or a chemical or physical procedure, for example an electroporation. Contacting a cell with a nucleic acid molecule provided herein also includes any method of artificially expressing a shRNA or other double-stranded RNA in a target cell by any method known in the art of transgene expression, resulting in the production of any nucleic acid provided herein, for example by processing of the shRNA or other double-stranded RNA to a siRNA by dicer.

As used herein, the term “composition” refers to an admixture of at least two components. A composition, accordingly, may comprise, for example, a nucleic acid molecule provided herein, or a pharmaceutically acceptable salt thereof, and a solvent (e.g., water). A composition may further comprise other components, for example, a buffer, a diluent, an exipient, a carrier, a transfection reagent, an emulsifier, and adjuvant, etc.

As used herein, the term “subject” refers to a human, non-human primate, or other mammal, for example a cow, horse, pig, sheep, goat, dog, cat or rodent.

As used herein, the term “pharmaceutical composition” refers to a composition comprising a pharmaceutically active ingredient, for example a nucleic acid molecule provided herein, and is suitable for administration to a mammalian subject. A pharmaceutical composition may include a nucleic acid molecule provided herein in a pharmaceutically acceptable form, for example in a form characterized by a certain bioavailability, half-life, toxicity to malignant cells, and/or lack of toxicity to non-malignant cells. A pharmaceutical composition may include an active ingredient, for example an ingredient that prevents or reduces the symptoms of a particular disease, and/or inhibits its progression. For example a pharmaceutical composition may be a composition that prevents and/or reduces the symptoms of a proliferative disease or disorder or that attenuates the progression of such a disorder (e.g., tumor growth and/or metastasis formation). It is contemplated that a pharmaceutical composition of the present invention will be provided in a form suitable for administration to a subject. The specific formulation of a pharmaceutical composition will depend on a number of factors, including the mode of administration. A pharmaceutical composition may contain diluents, adjuvants and excipients, among other ingredients.

In some embodiments, a pharmaceutical composition of this invention comprises a nucleic acid molecule as provided herein. In some embodiments, a pharmaceutical composition of this invention comprises a pharmaceutically acceptable salt of a nucleic acid molecule as provided herein. As used herein, the term “pharmaceutically acceptable salt” refers to those salts which are, within the scope of sound medical judgment, suitable for use in contact with the tissues of human or non-human subjects without undue toxicity, irritation, immunological response, and the like, and are commensurate with a reasonable benefit/risk ratio. Pharmaceutically acceptable salts are well known in the art. For example, Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences (1990), incorporated herein by reference). The use of any carrier compatible with the biological activity of a nucleic acid molecule as provided herein in the therapeutic or pharmaceutical compositions is contemplated.

As used herein, the terms treat, treated, or treating when used with respect to a disorder such as cancer refers to a prophylactic treatment which increases the resistance of a subject to development of the disease or, in other words, decreases the likelihood that the subject will develop the disease as well as a treatment after the subject has developed the disease in order to fight the disease, prevent the disease from becoming worse, or slow the progression of the disease compared to in the absence of the therapy. In the context of some aspects of this invention, the term includes, but is not limited to, prevention or alleviation of symptoms, diminishment of extent of disease, stabilized (e.g., not worsening) state of disease, delay or slowing of disease progression, amelioration or palliation of the disease state, and remission (whether partial or total), whether detectable or undetectable. One of skill in the art realizes that a treatment may improve the disease condition in comparison to no treatment, but may not be a complete cure for the disease.

As used herein, the term “tumor” refers to a population of cells that displays aberrant proliferation. A tumor is generally the manifestation of a cancer. Both solid and non-solid tumors are included in the scope of the term as used herein.

In some embodiments, a target cell, for example a human cancer cell, is contacted with a PKM2 inhibitory nucleic acid molecule or a composition including such a molecule as provided herein.

In some embodiments, delivery of a PKM2 inhibitory nucleic acid molecule into a target cell, for example a human cancer cell, is enhanced by formulating the molecule with a reagent that enhances cellular uptake using suitable art recognized methods, including, for example calcium phosphate, DMSO, glycerol or dextran, or by electroporation, or by transfection, e.g., using cationic, anionic, or neutral lipid compositions or liposomes using methods known in the art (see e.g., WO 90/14074; WO 91/16024; WO 91/17424; U.S. Pat. No. 4,897,355; Bergan et al. 1993. Nucleic Acids Research. 21:3567). Enhanced delivery of nucleic acid molecules can also be mediated by the use of vectors (See e.g., Shi, Y. 2003. Trends Genet. 2003 Jan. 19:9; Reichhart J M et al. Genesis. 2002. 34(1-2):1604, Yu et al. 2002. Proc. Natl. Acad. Sci. USA 99:6047; Sui et al. 2002. Proc. Natl. Acad. Sci. USA 99:5515) viruses, polyamine or polycation conjugates using compounds such as polylysine, protamine, or Ni, N12-bis(ethyl) spermine (see, e.g., Bartzatt, R. et al. 1989. Biotechnol. Appl. Biochem. 11:133; Wagner E. et al. 1992. Proc. Natl. Acad. Sci. 88:4255).

In some embodiments, a PKM2 inhibitory nucleic acid molecule provided by the invention is delivered to a target cell by using a beta-glucan containing particles, such as those described in US 2005/0281781 A1, WO 2006/007372, and WO 2007/050643 (all incorporated herein by reference). In some embodiments, the beta-glucan particle is derived from yeast. In some embodiments, the payload trapping molecule is a polymer, such as those with a molecular weight of at least about 1000 Da, 10,000 Da, 50,000 Da, 100 kDa, 500 kDa, etc. Preferred polymers include (without limitation) cationic polymers, chitosans, or PEI (polyethylenimine). In some embodiments, a beta-glucan based delivery system is provided that is formulated for oral delivery. In some embodiments, the orally delivered beta-glucan construct may be engulfed by macrophages or other related phagocytic cells, which may in turn release the subject constructs in selected in vivo sites.

The specific method for delivery of a PKM2 inhibitory nucleic acid molecule as provided herein to a target cell will depend upon a number of factors, for example, the type of cells that are being targeted, the nature and concentration/dosage of the nucleic acid molecule to be delivered, the delivery setting (e.g., in vivo, ex vivo, in vitro), the route of administration, and, for in vitro applications, the type of culture the cells are in (e.g., a suspension culture or plated) and the type of media in which the cells are grown.

In some embodiments, a PKM2 inhibitory nucleic acid molecule as provided by this invention is conjugated, for example, covalently bound, to a conjugating agent. In some embodiments, a PKM2 inhibitory nucleic acid molecule is provided that is derivatized or chemically modified by binding to a conjugating agent to facilitate cellular uptake. For example, covalent linkage of a cholesterol moiety to a nucleic acid molecule can improve cellular uptake by 5- to 10-fold which in turn improves DNA binding by about 10-fold (Boutorin et al., 1989, FEBS Letters 254:129-132). Conjugation of octyl, dodecyl, and octadecyl residues enhances cellular uptake by 3-, 4-, and 10-fold as compared to unmodified nucleic acid molecules (Vlassov et al., 1994, Biochimica et Biophysica Acta 1197:95-108). Similarly, derivatization of nucleic acid molecules with poly-L-lysine can aid oligonucleotide uptake by cells (Schell, 1974, Biochem. Biophys. Acta 340:323, and Lemaitre et al., 1987, Proc. Natl. Acad. Sci. USA 84:648).

Certain protein carriers can also facilitate cellular uptake of PKM2 inhibitory nucleic acid molecules, including, for example, serum albumin, nuclear proteins possessing signals for transport to the nucleus, and viral or bacterial proteins capable of cell membrane penetration. Therefore, protein carriers are useful when associated with or linked to PKM2 inhibitory nucleic acid molecules as provided herein. Accordingly, the present invention provides for derivatization of PKM2 inhibitory nucleic acid molecules with groups capable of facilitating cellular uptake, including hydrocarbons and non-polar groups, cholesterol, long chain alcohols (i.e., hexanol), poly-L-lysine, and proteins, as well as other aryl or steroid groups and polycations having analogous beneficial effects, such as phenyl or naphthyl groups, quinoline, anthracene or phenanthracene groups, fatty acids, fatty alcohols and sesquiterpenes, diterpenes, and steroids. A major advantage of using conjugating agents is to increase the initial membrane interaction that leads to a greater cellular accumulation of oligonucleotides.

Conjugating agents that may be used with the instantly provided PKM2 inhibitory nucleic acid molecules include those described in WO04048545A2 and US20040204377A1, such as a Tat peptide, a sequence substantially similar to the sequence of SEQ ID NO: 12 of WO04048545A2 and US20040204377A1, a homeobox (hox) peptide, a MTS, VP22, MPG, and at least one dendrimer (such as PAMAM).

Other conjugating agents that may be used with the instantly provided PKM2 inhibitory nucleic acid molecules include those described in WO07089607A2, which describes various nanotransporters and delivery complexes for use in delivery of nucleic acid molecules in vivo and in vivo. In some embodiments, a nucleic acid molecule as provided herein is conjugated to or associated with a nanotransporter comprising a core conjugated with at least one functional surface group. The core may be a nanoparticle, such as a dendrimer (e.g., a polylysine dendrimer). The core may also be a nanotube, such as a single walled nanotube or a multi-walled nanotube. The surface group may be a lipid, or a cell type specific targeting moiety, for example, a peptide binding to a cell-type specific antigen. The lipid may be an oleoyl lipid or derivative thereof. Exemplary nanotransporters include NOP-7 or HBOLD.

In some embodiments, an encapsulating agent that entraps a PKM2 inhibitory nucleic acid molecule as provided herein within a vesicle is employed for cellular delivery of a nucleic acid molecule to a target cell, for example a cancer cell. In some embodiments, a PKM2 inhibitory nucleic acid molecule is associated with a carrier or vehicle, e.g., a liposome or micelle, although other carriers could be used, as would be appreciated by one skilled in the art. Liposomes are vesicles made of a lipid bilayer having a structure similar to biological membranes. Such carriers are used to facilitate the cellular uptake or targeting of a PKM2-inhibitory nucleic acid molecule, or improve the pharmacokinetic or toxicologic properties of such a molecule.

In some embodiments, a nucleic acid molecule as provided herein is administered encapsulated in a liposome. A nucleic acid molecule as provided herein, depending upon solubility, may be present both in the aqueous layer and in the lipidic layer, or in what is generally termed a liposomic suspension. The hydrophobic layer, generally but not exclusively, comprises phopholipids such as lecithin and sphingomyelin, steroids such as cholesterol, more or less ionic surfactants such as diacetylphosphate, stearylamine, or phosphatidic acid, or other materials of a hydrophobic nature. In some embodiments, the diameters of the liposomes range from about 15 nm to about 5 micrometers.

The use of liposomes as drug delivery vehicles offers several advantages. Liposomes increase intracellular stability, increase uptake efficiency and improve biological activity. Liposomes are hollow spherical vesicles composed of lipids arranged in a similar fashion as those lipids which make up the cell membrane. They have an internal aqueous space for entrapping water soluble compounds and range in size from 0.05 to several microns in diameter. Several studies have shown that liposomes can deliver nucleic acids to cells and that the nucleic acids remain biologically active. For example, a lipid delivery vehicle originally designed as a research tool, such as Lipofectin or LIPOFECTAMINE™ 2000, can deliver intact nucleic acid molecules to cells.

Specific advantages of using liposomes for the delivery of PKM2 inhibitory nucleic acid molecules include the following: they are non-toxic and biodegradable, they display long circulation half-lives, and recognition molecules can be readily attached to their surface for targeting to tissues. For example, if the nature of a target cell, for example a cancer cell expressing PKM2, has been determined, it is possible to attach a binding agent specifically recognizing a surface antigen characteristic for the target cell. Surface antigens have been characterized for various types of cancer cells and suitable binding agents for such cancer-cell specific antigens are known in the art. (See e.g., Hood J D, Bednarski M, Frausto R, Guccione S, Reisfeld R A, Xiang R, Cheresh D A. Tumor regression by targeted gene delivery to the neovasculature. Science. 2002 Jun. 28; 296(5577):2404-7; Teesalu T, Sugahara K N, Kotamraju V R, Ruoslahti E. C-end rule peptides mediate neuropilin-1-dependent cell, vascular, and tissue penetration. Proc Natl Acad Sci USA. 2009 Sep. 22; 106(38):16157-62; Patra C R, Bhattacharya R, Wang E, Katarya A, Lau J S, Dutta S, Muders M, Wang S, Buhrow S A, Safgren S L, Yaszemski M J, Reid J M, Ames M M, Mukherjee P, Mukhopadhyay D. Targeted delivery of gemcitabine to pancreatic adenocarcinoma using cetuximab as a targeting agent. Cancer Res. 2008 Mar. 15; 68(6):1970-8; McNamara J O 2nd, Andrechek E R, Wang Y, Viles K D, Rempel R E, Gilboa E, Sullenger B A, Giangrande P H. Cell type-specific delivery of siRNAs with aptamer-siRNA chimeras. Nat. Biotechnol. 2006 August; 24(8):1005-15; Conaghan P, Ashraf S, Tytherleigh M, Wilding J, Tchilian E, Bicknell D, Mortensen N J, Bodmer W. Targeted killing of colorectal cancer cell lines by a humanized IgG1 monoclonal antibody that binds to membrane-bound carcinoembryonic antigen. Br J. Cancer. 2008 Apr. 8; 98(7):1217-25; Cirstoiu-Hapca A, Bossy-Nobs L, Buchegger F, Gurny R, Delie F. Differential tumor cell targeting of anti-HER2 (Herceptin) and anti-CD20 (Mabthera) coupled nanoparticles. Int J. Pharm. 2007 Mar. 1; 331(2):190-6; Chowdhury P S, Viner J L, Beers R, Pastan I. Isolation of a high-affinity stable single-chain Fv specific for mesothelin from DNA-immunized mice by phage display and construction of a recombinant immunotoxin with anti-tumor activity. Proc Natl Acad Sci USA. 1998 Jan. 20; 95(2):669-74; Daniels D A, Chen H, Hicke B J, Swiderek K M, Gold L. A tenascin-C aptamer identified by tumor cell SELEX: systematic evolution of ligands by exponential enrichment. Proc Natl Acad Sci U S A. 2003 Dec. 23; 100(26):15416-21; and Hu-Lieskovan S, Heidel J D, Bartlett D W, Davis M E, Triche T J. Sequence-specific knockdown of EWS-FLI1 by targeted, nonviral delivery of small interfering RNA inhibits tumor growth in a murine model of metastatic Ewing's sarcoma. Cancer Res. 2005 Oct. 1; 65(19):8984-92; all of which are incorporated herein in their entirety by reference for disclosure of cancer-cell specific antigens and suitable binding agents).

In some embodiments, a PKM2-inhibitory nucleic acid molecule as provided herein is formulated into a composition with a complexing agent, for example, a cationic lipid. Complexing agents bind to nucleic acid molecule, for example those provided herein, via non-covalent interaction (e.g., an electrostatic, van der Waals, pi-stacking, etc. interaction) and such complexes are efficiently taken up by target cells. Cationic lipids useful for delivery of nucleic acid molecules to a target cell are known in the art, and, generally comprise both polar and non-polar domains, are positively charged at physiological pH, bind to polyanions, such as nucleic acid molecules, and facilitate the delivery of nucleic acids into cells. Examples of useful cationic lipids include polyethylenimine, polyamidoamine (PAMAM) starburst dendrimers, Lipofectin (a combination of DOTMA and DOPE), Lipofectase, LIPOFECTAMINE™ (e.g., LIPOFECTAMINE™ 2000), DOPE, Cytofectin (Gilead Sciences, Foster City, Calif.), and Eufectins (JBL, San Luis Obispo, Calif.). Exemplary cationic liposomes can be made from N-[1-(2,3-dioleoloxy)-propyl]-N,N,N-trimethylammonium chloride (DOTMA), N-[1-(2,3-dioleoloxy)-propyl]-N,N,N-trimethylammonium methylsulfate (DOTAP), 3β-[N—(N′,N′-dimethylaminoethane)carbamoyl] cholesterol (DC-Chol), 2,3,-dioleyloxy-N-[2(sperminecarboxamido)ethyl]-N,N-dimethyl-1-propanaminium trifluoroacetate (DOSPA), 1,2-dimyristyloxypropyl-3-dimethyl-hydroxyethyl ammonium bromide; and dimethyldioctadecylammonium bromide (DDAB). The cationic lipid N-(1-(2,3-dioleyloxy)propyl)-N,N,N-trimethylammonium chloride (DOTMA), for example, was found to increase 1000-fold the antisense effect of a phosphorothioate oligonucleotide. (Vlassov et al., 1994, Biochimica et Biophysica Acta 1197:95-108). In some embodiments, PKM2 inhibitory nucleic acid molecules are complexed with, poly (L-lysine), or a derivative thereof, or avidin.

Cationic lipids have been used in the art to deliver nucleic acid molecules to cells (see, e.g., U.S. Pat. Nos. 5,855,910; 5,851,548; 5,830,430; 5,780,053; 5,767,099; Lewis et al. 1996. Proc. Natl. Acad. Sci. USA 93:3176; Hope et al. 1998. Molecular Membrane Biology 15:1). In addition, other lipid compositions are also known in the art and include, e.g., those taught in U.S. Pat. No. 4,235,871; U.S. Pat. Nos. 4,501,728; 4,837,028; 4,737,323.

In some embodiments, lipid compositions of the nucleic acid molecules provided herein further comprise other agents, e.g., viral proteins to enhance lipid-mediated transfections of oligonucleotides (Kamata, et al., 1994. Nucl. Acids. Res. 22:536). In some embodiments, a nucleic acid molecule is delivered to a target cell as part of a composition that also comprises a peptide and a lipid as taught, e.g., in U.S. Pat. No. 5,736,392. Improved lipids have also been described which are serum resistant (Lewis, et al., 1996. Proc. Natl. Acad. Sci. 93:3176).

In some embodiments, N-substituted glycine oligonucleotides (peptoids) is used to optimize uptake of oligonucleotides. Peptoids are well known in the art and have been used to create cationic lipid-like compounds for transfection (Murphy, et al., 1998. Proc. Natl. Acad. Sci. 95:1517). Peptoids can be synthesized using standard methods (e.g., Zuckermann, R. N., et al. 1992. J. Am. Chem. Soc. 114:10646; Zuckermann, R. N., et al. 1992. Int. J. Peptide Protein Res. 40:497). Combinations of cationic lipids and peptoids, liptoids, can also be used to optimize uptake of the nucleic acid molecules provided herein (Hunag, et al., 1998. Chemistry and Biology. 5:345). Liptoids can be synthesized by elaborating peptoid nucleic acid molecules and coupling the amino terminal submonomer to a lipid via its amino group (Hunag, et al., 1998. Chemistry and Biology. 5:345).

It is known in the art that positively charged amino acids can be used for creating highly active cationic lipids (Lewis et al. 1996. Proc. Natl. Acad. Sci. U.S.A. 93:3176). In some embodiments, a composition for delivering PKM2-inhibitory nucleic acid molecules to a target cell comprises a number of arginine, lysine, histidine or ornithine residues linked to a lipophilic moiety (see e.g., U.S. Pat. No. 5,777,153).

In some embodiments, a composition for delivering a PKM2-inhibitory nucleic acid as provided herein comprises a peptide having from between about one to about four basic residues. These basic residues can be located, e.g., on the amino terminal, C-terminal, or internal region of the peptide. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine (can also be considered non-polar), asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Apart from the basic amino acids, a majority or all of the other residues of the peptide can be selected from the non-basic amino acids, e.g., amino acids other than lysine, arginine, or histidine.

In some embodiments, a composition for delivering a PKM2 inhibitory nucleic acid molecule provided herein comprises a natural or synthetic polypeptide having one or more gamma carboxyglutamic acid residues, or γ-Gla residues. These gamma carboxyglutamic acid residues enable the polypeptide to bind to membrane surfaces. The gamma carboxyglutamic acid residues may exist in natural proteins (for example, prothrombin has 10γ-Gla residues). Alternatively, they can be introduced into purified, recombinantly produced, or chemically synthesized polypeptides by carboxylation using, for example, a vitamin K-dependent carboxylase. The gamma carboxyglutamic acid residues may be consecutive or non-consecutive, and the total number and location of such gamma carboxyglutamic acid residues in the polypeptide can be regulated to achieve different levels of “stickiness” of the polypeptide and the associated PKM2-inhibitory nucleic acid.

In some embodiments, a nucleic acid molecule as provided herein is modified by attaching a peptide sequence that transports the nucleic acid molecule into a target cell. Such transporting peptides are well known in the art, and examples of such transporting peptides include, e.g., HIV TAT proteins, lactoferrin, Herpes VP22 protein, and fibroblast growth factor 2 (Pooga et al. 1998. Nature Biotechnology. 16:857; and Derossi et al. 1998. Trends in Cell Biology. 8:84; Elliott and O'Hare. 1997. Cell 88:223). In some embodiments, PKM2-inhibitory nucleic acid molecules provided herein are attached to a transporting peptide using known techniques, e.g., (Prochiantz, A. 1996. Curr. Opin. Neurobiol. 6:629; Derossi et al. 1998. Trends Cell Biol. 8:84; Troy et al. 1996. J. Neurosci. 16:253), Vives et al. 1997. J. Biol. Chem. 272:16010). In some embodiments, a nucleic acid molecule provided herein bearing an activated thiol group is linked via that thiol group to a cysteine present in a transport peptide (e.g., to the cysteine present in the β turn between the second and the third helix of the antennapedia homeodomain as taught, e.g., in Derossi et al. 1998. Trends Cell Biol. 8:84; Prochiantz. 1996. Current Opinion in Neurobiol. 6:629; Allinquant et al. 1995. J. Cell Biol. 128:919). In some embodiments, a Boc-Cys-(Npys)OH group is coupled to the transport peptide as the last (N-terminal) amino acid and a PKM2-inhibitory nucleic acid molecule bearing an SH group is coupled to the peptide (Troy et al. 1996. J. Neurosci. 16:253).

In some embodiments, a PKM2-inhibitory nucleic acid molecule is provided as a molecular conjugate able to utilize receptor-mediated endocytotic mechanisms for entering a target cell (see, e.g., Bunnell et al. 1992. Somatic Cell and Molecular Genetics. 18:559, and the references cited therein). In some embodiments, the conjugated PKM2-inhibitory nucleic acid enters a target cell, for example a cancer cell, via a cellular receptor specifically or prominently expressed on the cell surface of the target cell. Cellular receptor-targeting moieties are well known in the art and can be conjugated directly to a PKM2-inhibitory nucleic acid molecule as provided herein or attached to a carrier group (e.g., poly(L-lysine) or liposomes) linked to such a nucleic acid molecule by methods well known in the art or described herein. This method is well suited to deliver PKM2-inhibitory nucleic acid molecules to target cells that display specific receptor-mediated endocytosis.

For example, nucleic acid molecules suitable for RNAi, which are conjugated to 6-phosphomannosylated proteins, are internalized 20-fold more efficiently by cells expressing mannose 6-phosphate specific receptors than unconjugated nucleic acid molecules. In some embodiments, a nucleic acid molecule as provided herein is coupled to a ligand for a cellular receptor using a biodegradable linker. In some embodiments, the delivery construct is mannosylated streptavidin which forms a tight complex with biotinylated nucleic acid molecules. Mannosylated streptavidin is known to increase 20-fold the internalization of biotinylated nucleic acid molecules by target cells. (Vlassov et al. 1994. Biochimica et Biophysica Acta 1197:95-108).

In addition, specific ligands can be conjugated to the polylysine component of polylysine-based delivery systems. For example, transferrin-polylysine, adenovirus-polylysine, and influenza virus hemagglutinin HA-2 N-terminal fusogenic peptides-polylysine conjugates greatly enhance receptor-mediated DNA delivery in eucaryotic cells. Mannosylated glycoprotein conjugated to poly(L-lysine) in aveolar macrophages has been employed to enhance the cellular uptake of nucleic acid molecules. Liang et al. 1999. Pharmazie 54:559-566.

Because malignant cells have an increased need for essential nutrients such as folic acid and transferrin, these nutrients can be used to target oligonucleotides to cancerous cells. For example, in some embodiments, folic acid may be linked to poly(L-lysine)-conjugated PKM2-inhibitory nucleic acid molecule to enhance uptake in target cancer cells. Such enhanced cellular uptake has been described in promyelocytic leukemia (HL-60) cells and human melanoma (M-14) cells (Ginobbi et al. 1997. Anticancer Res. 17:29). In some embodiments, PKM2-inhibitory nucleic acid molecules are conjugated with liposomes coated with maleylated bovine serum albumin, folic acid, or ferric protoporphyrin IX, to enhance cellular uptake. Such enhanced cellular uptake has been described in murine macrophages, KB cells, and 2.2.15 human hepatoma cells (Liang et al. 1999. Pharmazie 54:559-566).

Nucleic acid molecules in liposomes accumulate in the liver, spleen, and reticuloendothelial system in a process called passive targeting. By coupling liposomes to various ligands such as antibodies or protein A, they can be actively targeted to specific cell populations. For example, protein A-bearing liposomes may be pretreated with H-2K specific antibodies which are targeted to the mouse major histocompatibility complex-encoded H-2K protein expressed on L cells. (Vlassov et al. 1994. Biochimica et Biophysica Acta 1197:95-108).

Other in vitro and/or in vivo delivery of RNAi reagents are known in the art, and can be used to deliver the subject RNAi constructs. See, for example, U.S. patent application publications 20080152661, 20080112916, 20080107694, 20080038296, 20070231392, 20060240093, 20060178327, 20060008910, 20050265957, 20050064595, 20050042227, 20050037496, 20050026286, 20040162235, 20040072785, 20040063654, 20030157030, WO 2008/036825, WO04/065601, and AU2004206255B2 (all of which are incorporated herein by reference).

In some embodiments, a target cell is contacted with a nucleic acid molecule provided herein for between about 1 hour to about 24 hours. In some embodiments, a target cell is contacted with a nucleic acid molecule provided herein for between about 1 day to about 5 days. In some embodiments, a target cell is contacted with a nucleic acid molecule provided herein for between about 5 days to about 10 days. In some embodiments, a target cell is contacted with a nucleic acid molecule provided herein for between about 7 days to about 14 days. In some embodiments, a target cell is contacted with a nucleic acid molecule provided herein for between about 14 days to about a month. In some embodiments, a target cell is contacted with a nucleic acid molecule provided herein for between about 1 month to about three months. In some embodiments, a target cell is contacted with a nucleic acid molecule provided herein for longer than three months. In some embodiments, a target cell is contacted with a nucleic acid molecule provided herein as part of a chronic application schedule.

### Administration of PKM2 Inhibitory Nucleic Acid Molecules to a Subject

Some aspects of the invention relate to methods of administering PKM2 inhibitory nucleic acid molecules as described herein to a subject, for example, a human subject having a cancer. In some embodiments, a PKM2-inhibitory nucleic acid molecule is administered to a subject to reduce PKM2 expression, reduce cell viability, or induce apoptosis in a target cell or cell population, for example a malignant cell or cell population. In general, PKM2-inhibitory nucleic acid molecules provided herein are administered to a subject formulated into a pharmaceutically acceptable composition as described in more detail elsewhere herein.

The PKM2-inhibitory nucleic acid molecules provided herein can be administered to a subject, for example, a subject having cancer, via any route suitable for administering medications to a target cell or tissue. Depending upon the condition, for example, the type of cancer to be treated, a therapeutic nucleic acid molecule may be administered by a systemic route. Systemic routes include oral and parenteral routes, for example, intramuscular, intraperitoneal, and intravenous injection. In some embodiments, a more localized administration is preferred, for example via inhalation, topical application, vaginal, or rectal administration. In some embodiments, the PKM2-inhibitory nucleic acid molecules provided herein are administered directly to a target tissue or body fluid, for example a solid tumor, or the blood or bone marrow of a subject. Direct tissue administration may be achieved by direct injection.

In some embodiments, for example in embodiments where the target tissue is a tissue of the respiratory system, for example, in the treatment of a subject with a lung cancer, inhalation may be a preferred route for administration of a PKM2-inhibitory nucleic acid molecule. Several types of metered dose inhalers are regularly used for administration by inhalation. These types of devices include metered dose inhalers (MDI), breath-actuated MDI, dry powder inhaler (DPI), spacer/holding chambers in combination with MDI, and nebulizers.

In some embodiments, for example in embodiments where the target tissue is a skin tissue or a tissue underlying the skin, for example, in the treatment of a subject with a melanoma, or other form of skin cancer, topical administration may be a preferred route for administration of a PKM2-inhibitory nucleic acid molecule.

For oral administration, a therapeutic nucleic acid as provided herein can be formulated by combining it with pharmaceutically acceptable carriers well known in the art to be suitable for oral administration. Such carriers enable a PKM2-inhibitory nucleic acid to be formulated as tablets, pills, dragees, capsules, liquids, gels, syrups, slurries, suspensions and the like, for oral ingestion by a subject.

In some embodiments, a PKM2-inhibitory nucleic acid molecule, as provided herein is administered to a subject in the form of a liquid composition via an osmotic pump providing continuous infusion of, for example, as described in Rataiczak et al. (1992 Proc. Natl. Acad. Sci. USA 89:11823-11827). Such osmotic pumps are commercially available, e.g., from Alzet Inc. (Palo Alto, Calif.).

By inhibiting the expression of PKM2, the nucleic acid molecules and compositions provided by aspects of this invention can be used to treat any disease caused by or associated with overexpression of PKM2, for example, virtually any hyperproliferative disease, such as cancer, as described in more detail elsewhere herein.

In some embodiments, in vitro treatment of cells with a PKM2-inhibitory nucleic acid molecule as provided herein can be used for cells removed from a subject (e.g., for treatment of leukemia or viral infection) or for treatment of cells which did not originate in the subject, but are to be administered to the subject (e.g., to eliminate PKM2 expression in cells to be transplanted into a subject). In some embodiments, a cell from a subject is contacted in vitro or ex vivo with a siRNA as provided by aspects of this invention and the cell is returned to the subject as part of a cell replacement therapeutic approach. In some embodiments, a somatic cell is obtained from a subject and reprogrammed to a differentiation state different from the differentiation state of the original cell, for example, to a pluripotent state. In some embodiments, progeny of the reprogrammed cell, for example, a cell of a desired differentiation state, is administered to the subject the somatic cell was obtained from. In some embodiments, the somatic cell or its progeny is contacted with a siRNA according to aspects of this invention prior to administration of the cell or its progeny to the subject. In some embodiments, the cell is a pluripotent stem cell and the siRNA according to aspects of this invention is administered to mitigate the risk of tumorigenesis after administration of differentiated progeny of the pluripotent stem cell to a subject.

The dosage of a PKM2-inhibitory nucleic acid molecule to be administered to a subject and the particular mode of administration will vary depending upon such factors as the target cell type, the age, weight and health status of the particular subject, the particular nucleic acid molecule or compositions thereof, and the delivery method used. These are factors well known to those of ordinary skill in the art and can be addressed with no more than routine experimentation. It is preferred generally that a maximum dose be used, that is, the highest safe dose according to sound medical judgment.

In some embodiments, an initial dose is administered, the effect of the initial dose on the condition or disease of the subject is monitored, and the dose is adjusted according to observations made during the monitoring. The dosage of PKM2-inhibitory nucleic acids can be adjusted to optimally reduce expression of a protein translated from a target nucleic acid molecule, e.g., as measured by a readout of RNA stability or by a therapeutic response, without undue experimentation. For example, expression of PKM2 can be measured to determine whether or not the dosage regimen needs to be adjusted accordingly. In addition, an increase or decrease in PKM2 RNA or protein levels in a cell or produced by a cell can be measured using any art recognized technique. By determining whether expression has been decreased, the effectiveness of the oligonucleotide in inducing the cleavage of a target RNA can be determined. Assays for measuring expression, for example for assaying RNA or protein expression levels, are well known in the art.

In some embodiments, a PKM2-inhibitory nucleic acid molecule is administered to a subject diagnosed to bear a malignant cell population expressing, or suspected to express, PKM2, for example in the form of a solid or non-solid tumor. In some embodiments, the target cell population is quantified before administration of a therapeutic nucleic acid as provided herein, for example, by measuring the size of a solid tumor or by determining the number of malignant cells in a sample of tissue or body fluid from the subject. In some embodiments, an initial dose of a therapeutic, PKM2-inhibitory nucleic acid is then administered and, after a time sufficient for the therapeutic nucleic acid molecule to be delivered to the target cells and to effect a biological result, for example, induction of apoptosis or reduction of cell proliferation, the target cell population is quantified again. In some embodiments, the target cell quantity observed before administration is compared to the target cell quantity observed after administration of a therapeutic nucleic acid as provided herein and, if no desired effect is measured, for example, if the target cell quantity after administration is equal or higher than the quantity before administration, the dosage of therapeutic nucleic acid is increased.

Typically, an effective dosage, for example, a dosage effective to decrease cell viability or induce apoptosis in a target cancer cell population, is administered until a desired effect, for example, regression of a tumor, is achieved. In some embodiments, a single dose of a PKM2-inhibitory nucleic acid molecule is administered. In some embodiments, a plurality of doses of a PKM2-inhibitory nucleic acid molecule is administered. Dosage regimen may be adjusted to provide the optimum therapeutic response. For example, a PKM2-inhibitory nucleic acid molecule may be repeatedly administered, e.g., several doses may be administered, for example, once every 2-8 hours, once a day, once a week, or once a month, and the dose may be proportionally increased or reduced as indicated by the clinical situation. In some embodiments, a single administration of a PKM2-inhibitory nucleic acid may be sufficient to achieve a desired clinical effect. Examples of such embodiments are delivery of an expression construct expressing a PKM2-inhibitory nucleic acid molecule in a target cell, or administration of a composition resulting in the sustained release of a nucleic acid molecule as provided herein. One of ordinary skill in the art will readily be able to determine appropriate doses and schedules of administration of a nucleic acid molecule as provided herein, both for in vitro administration to cells in culture or to in vivo administration to a subject.

Doses of PKM2-inhibitory nucleic acids for in vivo administration typically range from about 1 nanogram to about 10,000 mg per kg subject body weight. Exemplary dosages, accordingly, range from about 1 nanogram, about 1 microgram, about 5 microgram, about 10 microgram, about 50 microgram, about 100 microgram, about 200 microgram, about 350 microgram, about 500 microgram, about 1 milligram, about 5 milligram, about 10 milligram, about 50 milligram, about 100 milligram, about 200 milligram, about 350 milligram, about 500 milligram, to about 1000 mg or more per kg subject body weight per administration, and any range derivable therein. In non-limiting examples of a derivable range from the numbers listed herein, a range of about 5 mg to about 100 mg, about 5 microgram to about 500 milligram, per kg subject body weight, etc., can be administered, based on the numbers described above.

In some embodiments, a composition of a PKM2-inhibitory nucleic acid molecule and a lipid, for example, a cationic lipid, is administered to a subject. In some such embodiments, the amount of cationic lipid compound administered is in the range from between about 0.1 microgram per kg body weight to about 1 gram per kg body weight.

In some embodiments, a PKM2-inhibitory nucleic acid molecule as provided herein is administered with another medicament, such as an anti-proliferative drug, for example a chemotherapeutic agent. In some such embodiments, a sub-therapeutic dosage of the nucleic acid molecule and/or the anti-neurological agent is administered, which is less than a dosage that would produce a therapeutic result in the subject if administered in the absence of the other agent. Therapeutic and sub-therapeutic doses of anti-proliferative drugs, for example chemotherapeutic agents, are well known in the art. These dosages have been extensively described in references such as Remington's Pharmaceutical Sciences, 18th ed., 1990; as well as many other medical references relied upon by the medical profession as guidance for the treatment of proliferative disorders.

Some aspects of this invention relate to compositions comprising a nucleic acid molecule able to inhibit expression of PKM2, as described herein. A PKM2-inhibitory nucleic acid molecule provided by this invention may be formulated into compositions in solid, semi-solid, liquid or gaseous forms, such as tablets, capsules, powders, granules, ointments, solutions, depositories, inhalants and injections. Some aspects of this invention also embrace pharmaceutical compositions which are formulated for local administration, for example, by sustained-release implants.

Some embodiments include the use of a PKM2-inhibitory nucleic acid molecule provided herein in the manufacture of a medicament. In some embodiments, a nucleic acid molecule provided herein is formulated into a pharmaceutical composition for administration to a subject. In some embodiments, a composition is provided that includes a nucleic acid able to inhibit PKM2 expression as described herein, and a pharmaceutically acceptable carrier. Pharmaceutically-acceptable carriers for nucleic acid molecules are well-known to those of skill in the art. As used herein, a pharmaceutically-acceptable carrier means a non-toxic material that does not interfere with the effectiveness of the biological activity of the active ingredients.

A composition according to some aspects of this invention may comprise different types of carriers depending on whether it is to be administered in solid, liquid or aerosol form, and whether it need to be sterile for such routes of administration as injection.

The PKM2-inhibitory nucleic acid molecules provided by aspects of this invention can be administered intravenously, intradermally, intraarterially, intralesionally, intratumorally, intracranially, intraarticularly, intraprostaticaly, intrapleurally, intratracheally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, orally, topically, locally, inhalation (e.g., aerosol inhalation), injection, infusion, continuous infusion, localized perfusion bathing target cells directly, via a catheter, via a lavage, in creams, in lipid compositions (e.g., liposomes), or by other method or any combination of the forgoing as would be known to one of ordinary skill in the art (see, for example, Remington's Pharmaceutical Sciences (1990), incorporated herein by reference).

Optionally the compositions may be sterile.

In some embodiments, the composition is in a liquid form, and the carrier can be a solvent or dispersion medium comprising but not limited to, water, ethanol, polyol (e.g., glycerol, propylene glycol, liquid polyethylene glycol, etc.), lipids (e.g., triglycerides, vegetable oils, liposomes) and combinations thereof. Proper fluidity of liquid compositions can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In some embodiments, a liquid composition is provided that includes an isotonic agent, for example, a sugar, a salt, e.g., sodium chloride, or combinations thereof.

In some embodiments, the nucleic acid molecules provided herein are delivered systemically to a subject. In some such embodiments, the nucleic acid molecules are formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

In some embodiments, in which the nucleic acid molecules are used therapeutically, a desirable route of administration may be by pulmonary aerosol. Techniques for preparing aerosol delivery systems containing compounds are well known to those of skill in the art.

Generally, such systems should utilize components which will not significantly impair the PKM2 expression inhibiting properties of a nucleic acid as provided herein (see, for example, Sciarra and Cutie, “Aerosols,” in Remington's Pharmaceutical Sciences, 18th edition, 1990, pp 1694-1712; incorporated by reference). Those of skill in the art can readily determine the various parameters and conditions for producing aerosols without resort to undue experimentation.

In some embodiments, a composition of a PKM2-inhibitory nucleic acid molecule is formulated as microemulsions. A microemulsion is a system of water, oil and amphiphile which is a single optically isotropic and thermodynamically stable liquid solution. Typically microemulsions are prepared by first dispersing an oil in an aqueous surfactant solution and then adding a sufficient amount of a 4th component, generally an intermediate chain-length alcohol to form a transparent system. Surfactants that may be used in the preparation of microemulsions include, but are not limited to, ionic surfactants, non-ionic surfactants, Brij 96, polyoxyethylene oleyl ethers, polyglycerol fatty acid esters, tetraglycerol monolaurate (ML310), tetraglycerol monooleate (MO310), hexaglycerol monooleate (PO310), hexaglycerol pentaoleate (PO500), decaglycerol monocaprate (MCA750), decaglycerol monooleate (MO750), decaglycerol sequioleate (S0750), decaglycerol decaoleate (DA0750), alone or in combination with cosurfactants. The cosurfactant, usually a short-chain alcohol such as ethanol, 1-propanol, and 1-butanol, serves to increase the interfacial fluidity by penetrating into the surfactant film and consequently creating a disordered film because of the void space generated among surfactant molecules. Microemulsions may be prepared without the use of cosurfactants and alcohol-free self-emulsifying microemulsion systems are known in the art. The aqueous phase may typically be, but is not limited to, water, an aqueous solution of the drug, glycerol, PEG300, PEG400, polyglycerols, propylene glycols, and derivatives of ethylene glycol. The oil phase may include, but is not limited to, materials such as Captex 300, Captex 355, Capmul MCM, fatty acid esters, medium chain (C₈-C₁₂) mono, di, and tri-glycerides, polyoxyethylated glyceryl fatty acid esters, fatty alcohols, polyglycolized glycerides, saturated polyglycolized C₈-C₁₀ glycerides, vegetable oils and silicone oil.

Microemulsions offer improved solubilization, protection from enzymatic hydrolysis, possible enhancement of absorption due to surfactant-induced alterations in membrane fluidity and permeability, ease of preparation, ease of oral administration over solid dosage forms, improved clinical potency, and decreased toxicity (Constantinides et al., Pharmaceutical Research, 1994, 11:1385; Ho et al., J. Pharm. Sci., 1996, 85:138-143). Microemulsions have also been effective in transdermal delivery in both cosmetic and pharmaceutical applications. It is expected that microemulsion compositions and formulations of nucleic acid molecules as provided herein will facilitate systemic absorption of oligonucleotides from the gastrointestinal tract, as well as improve local cellular uptake within the gastrointestinal tract, vagina, buccal cavity and other areas of administration.

In some embodiments, a composition of a PKM2-inhibitory nucleic acid molecule is formulated to effect efficient delivery of nucleic acid molecules as provided herein to the skin and underlying tissues of a subject. Generally, compositions for topical administration comprise a penetration enhance. Examples of penetration enhancers that may be used in the present invention include: surfactants, fatty acids, bile salts, chelating agents, and non-chelating non-surfactants. Other agents may be utilized to enhance the penetration of the administered PKM2-inhibitory nucleic acid molecules, including, for example: glycols such as ethylene glycol and propylene glycol, pyrrols such as 2-15 pyrrol, azones, and terpenes such as limonene, and menthone.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

Compositions suitable for oral administration may be presented as discrete units, such as capsules, tablets, lozenges, each containing a predetermined amount of the active agent. Other compositions include suspensions in aqueous liquids or non-aqueous liquids such as a syrup, elixir or an emulsion.

In some embodiments, the vehicle is a biocompatible microparticle or implant that is suitable for implantation into the mammalian recipient. Exemplary bioerodible implants that are useful in accordance with this method are described in PCT International Application No. PCT/US/03307 (Publication No. WO 95/24929, entitled “Polymeric Gene Delivery System”, claiming priority to U.S. patent application serial no. 213,668, filed Mar. 15, 1994). PCT/US/0307 describes a biocompatible, preferably biodegradable polymeric matrix for containing a biological macromolecule. The polymeric matrix may be used to achieve sustained release of a nucleic acid molecule as provided herein in a subject. In accordance with one aspect of the instant invention, a nucleic acid molecule as described herein may be encapsulated or dispersed within the biocompatible, preferably biodegradable polymeric matrix disclosed in PCT/US/03307. The polymeric matrix preferably is in the form of a microparticle such as a microsphere (wherein the nucleic acid molecule is dispersed throughout a solid polymeric matrix) or a microcapsule (wherein the nucleic acid molecule is stored in the core of a polymeric shell). Other forms of the polymeric matrix for containing the agent include films, coatings, gels, implants, and stents. The size and composition of the polymeric matrix device is selected to result in favorable release kinetics in the tissue into which the matrix device is implanted. The size of the polymeric matrix device further is selected according to the method of delivery which is to be used, typically injection into a tissue or administration of a suspension by aerosol into the nasal and/or pulmonary areas. The polymeric matrix composition can be selected to have both favorable degradation rates and also to be formed of a material which is bioadhesive, to further increase the effectiveness of transfer when the device is administered to a vascular, pulmonary, or other surface. The matrix composition also can be selected not to degrade, but rather, to release by diffusion over an extended period of time.

Both non-biodegradable and biodegradable polymeric matrices can be used to deliver the nucleic acid molecules described herein to the subject. Biodegradable matrices are preferred. Such polymers may be natural or synthetic polymers. Synthetic polymers are preferred. The polymer is selected based on the period of time over which release is desired, generally in the order of a few hours to a year or longer. Typically, release over a period ranging from between a few hours and three to twelve months is most desirable. The polymer optionally is in the form of a hydrogel that can absorb up to about 90% of its weight in water and further, optionally is cross-linked with multivalent ions or other polymers.

In general, the nucleic acid molecules of the invention may be delivered using the bioerodible implant by way of diffusion, or more preferably, by degradation of the polymeric matrix. Exemplary synthetic polymers which can be used to form the biodegradable delivery system include: polyamides, polycarbonates, polyalkylenes, polyalkylene glycols, polyalkylene oxides, polyalkylene terepthalates, polyvinyl alcohols, polyvinyl ethers, polyvinyl esters, poly-vinyl halides, polyvinylpyrrolidone, polyglycolides, polysiloxanes, polyurethanes and co-polymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), polyvinyl acetate, poly vinyl chloride, polystyrene and polyvinylpyrrolidone.

Examples of non-biodegradable polymers include ethylene vinyl acetate, poly(meth)acrylic acid, polyamides, copolymers and mixtures thereof.

Examples of biodegradable polymers include synthetic polymers such as polymers of lactic acid and glycolic acid, polyanhydrides, poly(ortho)esters, polyurethanes, poly(butic acid), poly(valeric acid), and poly(lactide-cocaprolactone), and natural polymers such as alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion.

Bioadhesive polymers of particular interest include bioerodible hydrogels described by H. S. Sawhney, C. P. Pathak and J. A. Hubell in Macromolecules, 1993, 26, 581-587, the teachings of which are incorporated herein, polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

Other delivery systems useful to administer the nucleic acids provided herein include time-release, delayed release or sustained release delivery systems. Such systems can avoid repeated administrations, increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono-di- and tri-glycerides; hydrogel release systems; silastic systems; peptide based systems; wax coatings; compressed tablets using conventional binders and excipients; and partially fused implants. Specific examples include, but are not limited to: (a) erosional systems in which the platelet reducing agent is contained in a form within a matrix such as those described in U.S. Pat. Nos. 4,452,775, 4,675,189, and 5,736,152 and (b) diffusional systems in which an active component permeates at a controlled rate from a polymer such as described in U.S. Pat. Nos. 3,854,480, 5,133,974 and 5,407,686. In addition, pump-based hardware delivery systems can be used, some of which are adapted for implantation.

Use of a long-term sustained release implant may be particularly suitable for treatment of chronic diseases. Long-term release, as used herein, means that the implant is constructed and arranged to delivery therapeutic levels of a nucleic acid provided by aspects of the invention for at least 30 days, and preferably 60 days. Long-term sustained release implants are well-known to those of ordinary skill in the art and include some of the release systems described above.

A composition comprising a nucleic acid provided herein may comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

### PKM-2 Inhibitory Nucleic Acid Molecule Kits

The PKM2-inhibitory nucleic acid molecules described herein may, in some embodiments, be assembled into pharmaceutical or diagnostic or research kits to facilitate their use in therapeutic, diagnostic or research applications. A kit may include one or more containers housing the components of the invention and instructions for use. Specifically, such kits may include one or more PKM2-inhibitory nucleic acid molecules described herein, along with instructions describing the intended therapeutic application and the proper administration of these molecules. In some embodiments, PKM2-inhibitory nucleic acid molecules in a kit may be in a pharmaceutical formulation and dosage suitable for a particular application and for a method of administration of the agents.

A kit comprising a PKM2-inhibitory nucleic acid molecule as provided herein may be designed to facilitate use of the methods described herein by physicians and can take many forms. Each of the compositions of the kit, where applicable, may be provided in liquid form (e.g., in solution), or in solid form, (e.g., a dry powder). In certain cases, some of the compositions may be constitutable or otherwise processable (e.g., to an active form), for example, by the addition of a suitable solvent or other species (for example, water or a cell culture medium), which may or may not be provided with the kit.

### EXAMPLES

### Materials and Methods

### Cell Culture.

Cells were maintained at 37° C. and 5% CO2 in Dulbecco's modified Eagle medium supplemented with L-glutamine, Penicillin/Streptomycin, and 10% fetal bovine serum (Hyclone).

siRNA Sequences.

Control siRNA (siLuciferase) target sequence:
(SEQ ID NO: 61)
5′-CTTACGCTGAGTACTTCGA-3′.

mRNA Quantitation.

15,000 cells per well were seeded in 96-well plates (Falcon) and transfected with 5 nM siRNA using HiPerFect Transfection Reagent (Qiagen). After 48 h, RNA was collected and reverse transcribed using the Power SYBR Green Cells-to-CT Kit (Ambion). Real-time PCR was performed on an Applied Biosystems 7300 using the following primer pairs (mismatches between M1 and M2 isoforms are underlined):

PKM1/M2 Forward Primer:
(SEQ ID NO: 62)
5′-CATTGATTCACCACCCATCA-3′.
PKM1/M2 Reverse Primer:
(SEQ ID NO: 63)
5′-AGACGAGCCACATTCATTCC-3′.
PKM1 Forward Primer:
(SEQ ID NO: 64)
5′-CGAGCCTCAAGTCACTCCAC-3′.
PKM1 Reverse Primer:
(SEQ ID NO: 65)
5′-GTGAGCAGACCTGCCAGACT-3′.
PKM2 Forward Primer:
(SEQ ID NO: 66)
5′-ATTATTTGAGGAACTCCGCCGCCT-3′.
PKM2 Reverse Primer:
(SEQ ID NO: 67)
5′-ATTCCGGGTCACAGCAATGATGG-3′.

### Western Blot Analysis.

250,000 cells per well were seeded in 6-well plates (Falcon) and transfected with 5 nM siRNA using HiPerFect Transfection Reagent (Qiagen). After 48 h, the cells were lysed, the supernatant was quantitated, and the protein was boiled in 4×SDS sample buffer, and run on 4%-12% gradient Bis-Tris gels (Invitrogen). Proteins were transferred to nitrocellulose, and Western blots performed using rabbit monoclonal anti-PKM1/M2 (Cell Signaling), rabbit polyclonal anti-PKM2 (Cell Signaling), and mouse monoclonal anti-Vinculin (Sigma).

Cell Viability Analysis. 5,000 cells per well were seeded in 96-well plates (Falcon) and transfected with 5 nM siRNA using HiPerFect Transfection Reagent (Qiagen). Cell viability was determined at 144 h by CellTiter-Glo® (Promega). To retain knockdown of PKM2, siRNA transfection was repeated at 48 h and 96 h.

### Assessment of Apoptosis.

5,000 cells per well were seeded in 96-well plates (Falcon) and transfected with 5 nM siRNA using HiPerFect Transfection Reagent (Qiagen). Relative apoptosis was determined by Caspase-Glo® 3/7 using the Apotox-Glo™ Assay (Promega). To retain knockdown of PKM2, siRNA transfection was repeated at 48 h and 96 h.

In Vivo siPKM2 Efficacy Study.

SCID mice (4 weeks old) were purchased from Taconic. All in vivo experimentation was carried out under the supervision of the Division of Comparative Medicine (DCM), Massachusetts Institute of Technology, and in compliance with the Principles of Laboratory Animal Care of the National Institutes of Health. HepG2 and SKOV3 were obtained from ATCC.

Anti-tumor efficacy was evaluated in SCID mice (3-4 weeks old, Taconic) induced with HepG2 and SKOV3 tumors on the left and right hind flanks, respectively, with a single s.c. injection of 2.5×10⁶ cells. Mice were randomized when the tumors were approximately ˜200 mm³ in volume and were subjected to treatment with either siControl or the indicated siRNA. The therapy was given in the form of an intratumoral injection twice a week at a dosage of ˜2 mg/kg/100 mm³ tumor volume. Measurements of tumor volume were made every 2 days using the equation: tumor volume=(width2×length)/2. The mice were monitored for up to 22 days and were euthanized when the tumor burden was deemed to be excessive by the DCM. One-way ANOVA tests at the 95% confidence interval were used for comparison of tumor sizes.

For histology, tumors were harvested from mice at the end of the trial (day 22), frozen in OCT (−80° C.) and cut into 5 μm sections for analysis. Sections were fixed (5% formaldehyde) and permeabilized with cold ethanol (70% volume). TUNEL assay was performed according to the manufacturer's instructions (Molecular Probes), and the nuclei were stained with Hoechst. Sections were examined with a Delta Vision confocal at 20×.

siRNA Nomenclature and Target Sequences for Examples 1-4

Si target sequences of siRNAs of Examples 1-4 are given below:

(SEQ ID NO: 14)
si1 target sequence: 5′-CCATAATCGTCCTCACCAA-3′.
(SEQ ID NO: 243)
si2 target sequence: 5′-CTTGCAATTATTTGAGGAA-3′.
(SEQ ID NO: 244)
si3 target sequence: 5′-TGCAATTATTTGAGGAACT-3′.
(SEQ ID NO: 245)
si4 target sequence: 5′-GCAGTGGGGCCATAATCGT-3′.
(SEQ ID NO: 246)
si5 target sequence: 5′-GTGAGGCAGAGGCTGCCAT-3′.
(SEQ ID NO: 247)
si6 target sequence: 5′-TCTACCACTTGCAATTATT-3′.
(SEQ ID NO: 248)
si7 target sequence: 5′-GGAGATGATTAAGTCTGGA-3′.
(SEQ ID NO: 249)
si8 target sequence: 5′-GGACCTGAGATCCGAACTG-3′.

### Example 1

### Identification of siRNAs that Confer Specific Knockdown of PKM2

Using every siRNA design algorithm that is publicly available, we attempted to identify sequences that would target the M2 isoform of pyruvate kinase specifically. Not one algorithm yielded an siRNA that targeted a sequence in exon 9 (M1) or exon 10 (M2) when given the entire pyruvate kinase muscle transcript as an input sequence. Upon narrowing down the input to exon 10 (M2), most algorithms did not yield a single hit. Only two sequences, designated si1 and si2, were suggested by more than one algorithm.

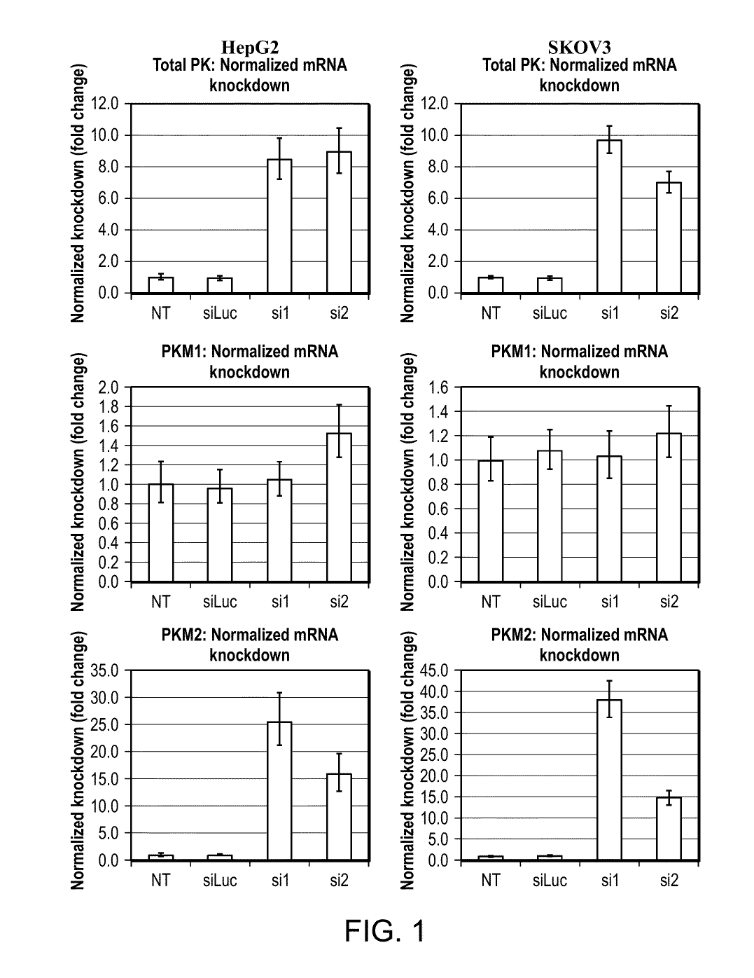

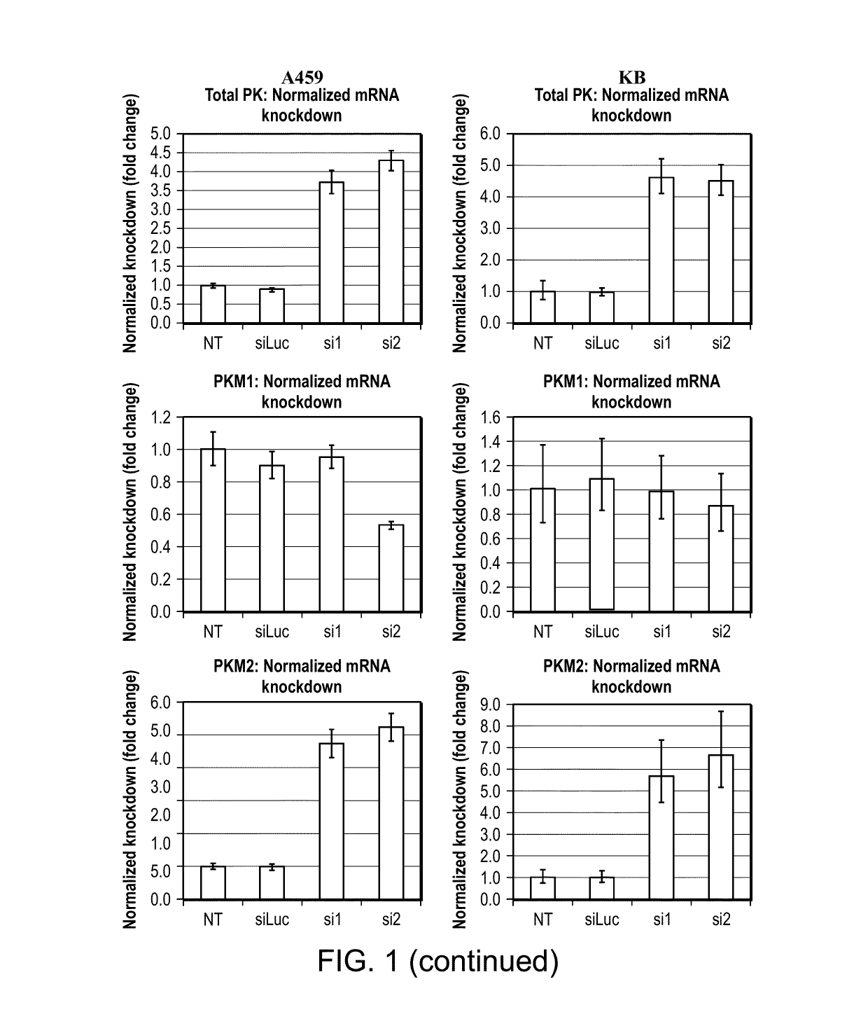

Four cell lines (HepG2, SKOV3, A549, and KB), representing four different cancer types (liver, ovarian, lung, and nasopharyngeal, respectively) were transfected with these two siRNAs. Real-time PCR was used to confirm that the knockdown was robust and specific (FIG. 1). With minimal effect on PKM1 levels, si1 conferred as much as 38-fold knockdown. si2 was moderately less specific and potent but still promising.

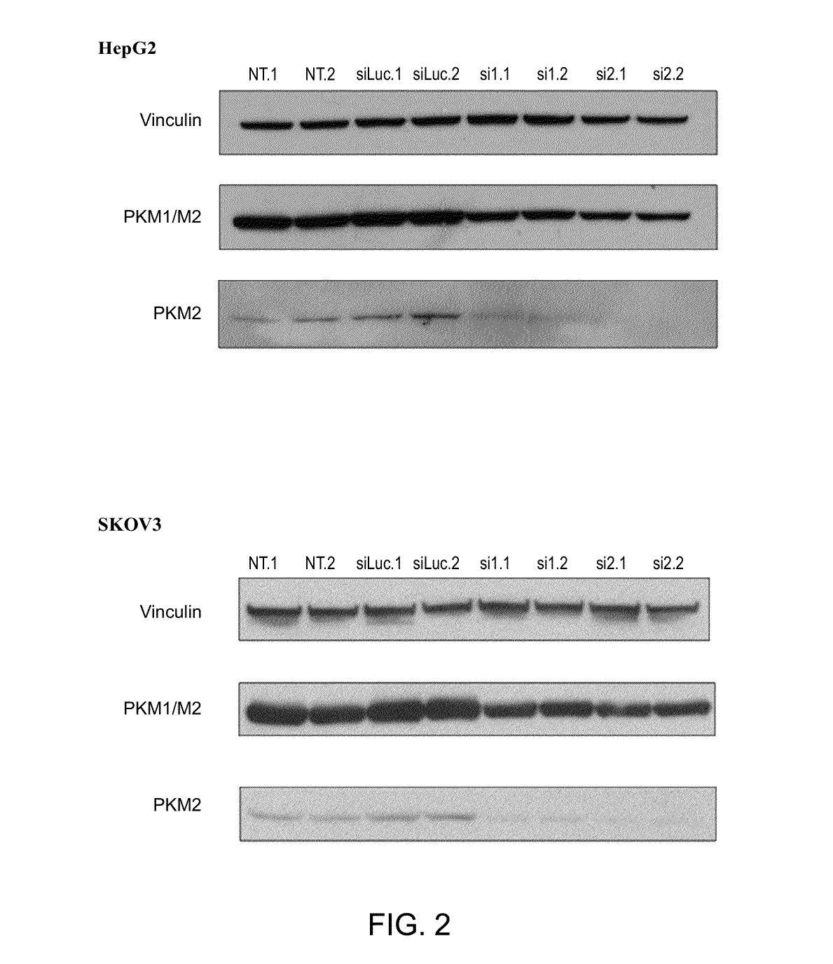

Because we had previously developed an siRNA delivery system that has been shown to be effective in the treatment of liver cancer and ovarian cancer, we elected to focus on HepG2 and SKOV3 for further analysis. Next, we confirmed that the knockdown was observable at the protein level (FIG. 2). Indeed, not only was total PK reduced but also PKM2 was specifically knocked down in both cell lines investigated.

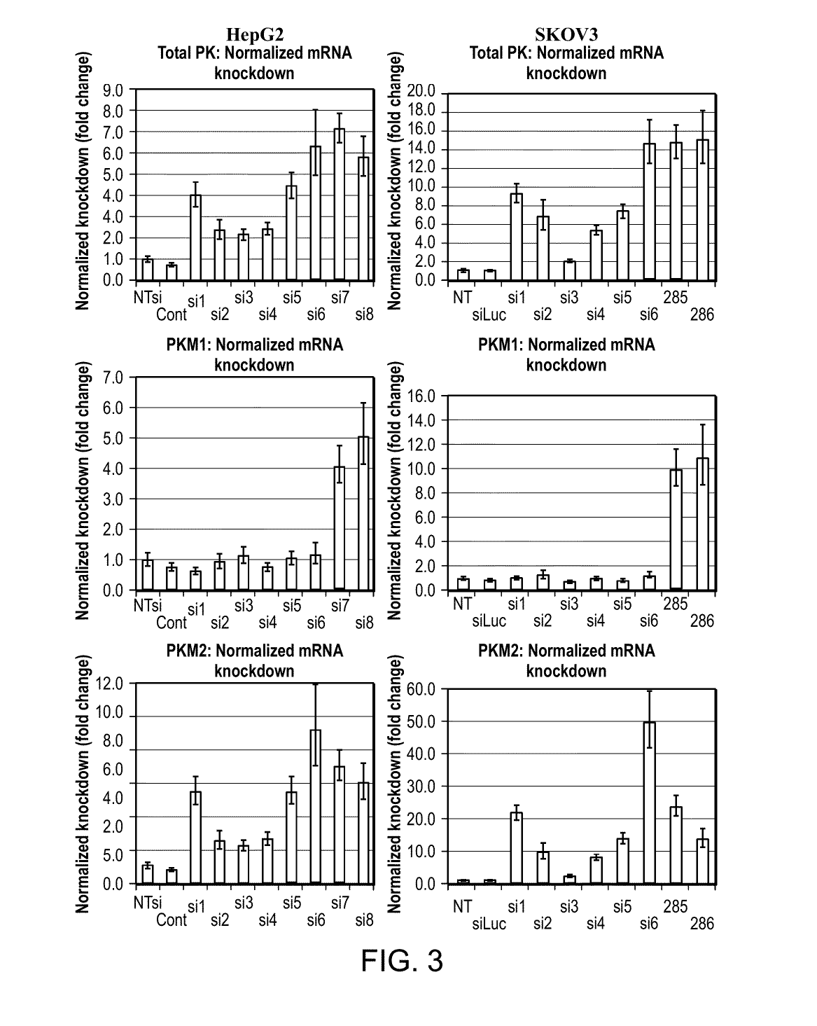

To elucidate the importance of algorithm design criteria, seed matching, and mismatch number and position, we selected four more 19-mers within exon 10 (M2), designated si3-sib, for analysis (see Materials and Methods for sequences, including positions of mismatches). We additionally included two commercially-available siRNAs, designated si7 and si8, as controls (every commercially-available siRNA against PKM2 that we found targets sequences that are common to both isoforms). We found that every siRNA screened was able to confer M2-specific knockdown, though the extent of knockdown varied between sequences (FIG. 3).

### Example 2

### siPKM2 Reduces Cell Viability and Induces Apoptosis

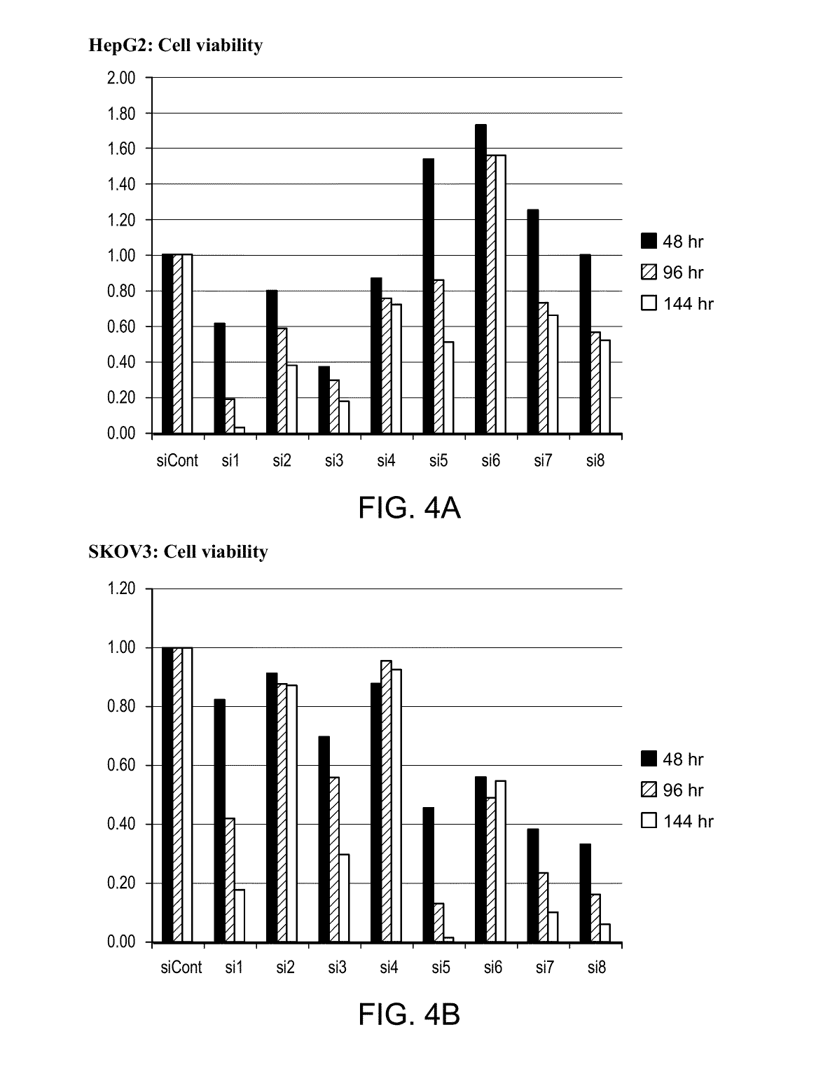

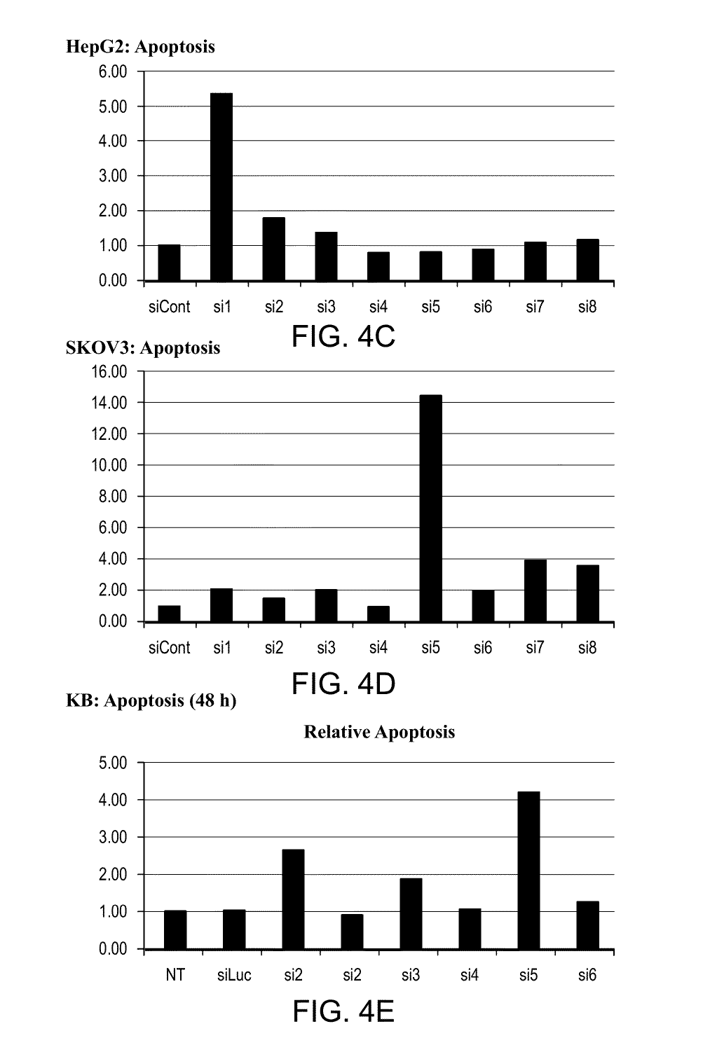

To confirm that the knockdown had an effect on phenotype, we examined cell proliferation (FIG. 4a,b). si1 and si5 afforded the greatest effect across the two cell lines, while si3, si7, and si8 also induced a marked decrease in viability. Importantly, this decreased relative viability could be attributed to apoptosis rather than simply inhibition of proliferation (FIG. 4c-e).

### Example 3

### si1 Leads to Profound Regression of Tumor Volume in Vivo

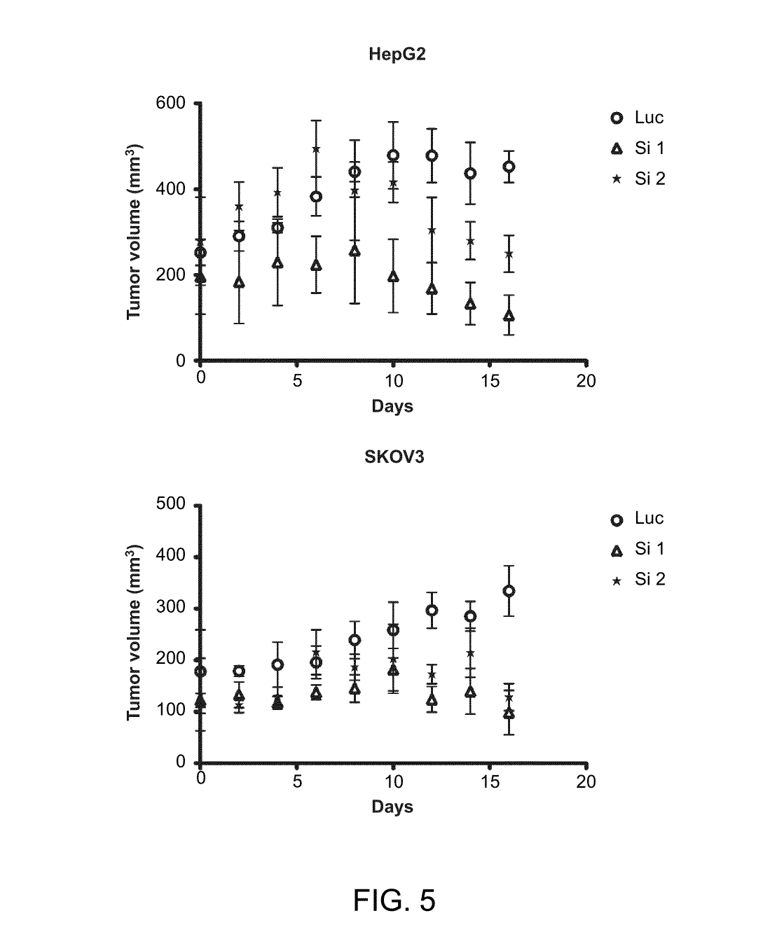

A member of the lipid-like class of materials dubbed “lipidoids”¹² was used to encapsulate and deliver siRNA to the xenografts. The selected lipidoid, ND98, has previously been shown to knock down claudin-3 in vivo, resulting in reduction of tumor volume and extension of survival in two mouse models of ovarian cancer¹³. HepG2 and SKOV3 cells were implanted as subcutaneous xenografts in scid mice. When tumors became established (>100 mm³), mice were treated with either si1 or siControl every 2 days for 2 weeks. The trial was halted when the control group had to be euthanized. Treatment with si1 prevented tumor expansion and even resulted in dramatic volume reduction (FIG. 5). Indeed, nearly all of the measured volume in the treated mice could be attributed to scar tissue caused by the intratumoral injections. There was no remnant of HepG2 tumor in half of the si1-treated animals, and there was no remnant of SKOV3 tumor in three quarters of the si1-treated animals.

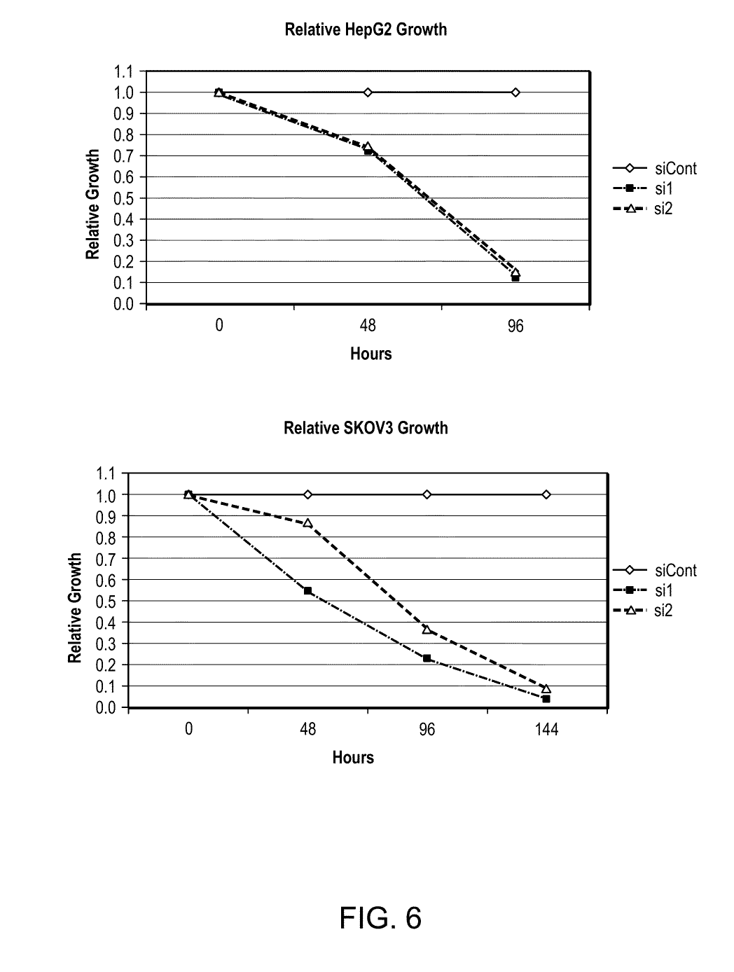

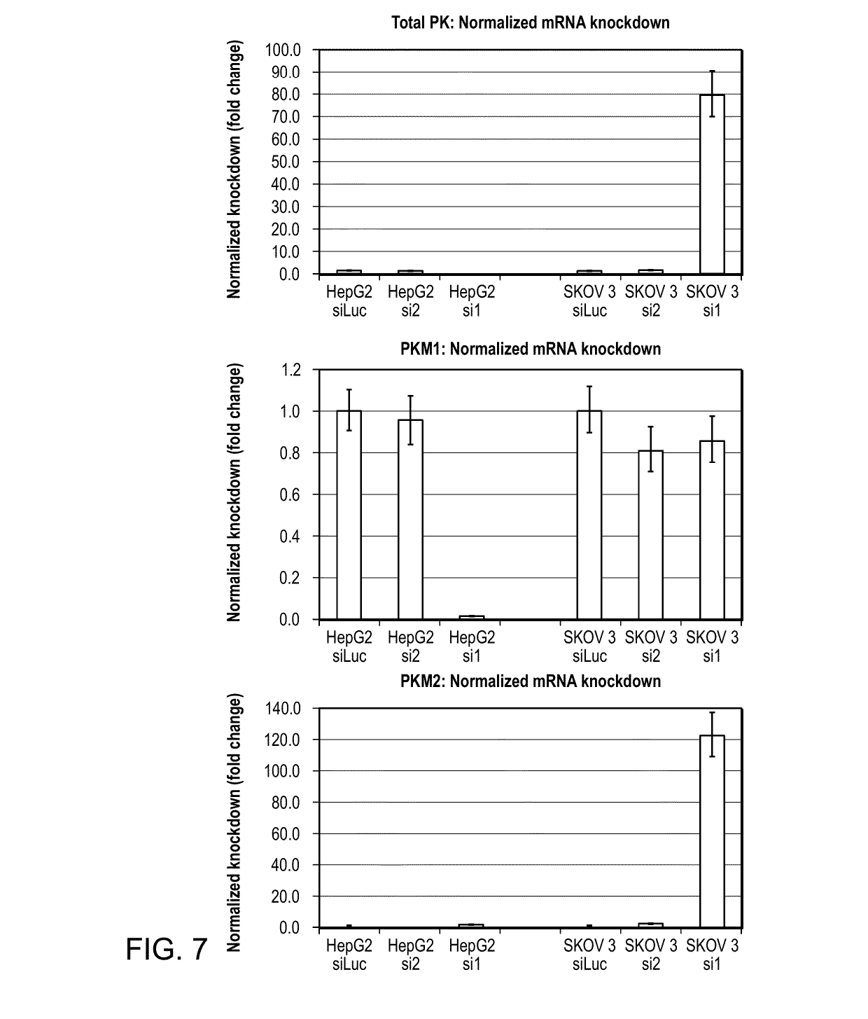

The tumors that were recovered were sectioned and visualized by TUNEL staining (FIG. 6). sit-treated tumors displayed significantly higher levels of apoptosis. RNA was extracted from the tumors, and real-time PCR was performed to examine specific knockdown of PKM2 in the SKOV3 xenograft (FIG. 7). Interestingly, in the HepG2 xenograft examined, PKM2 knockdown was modest, while PKM1 levels were dramatically upregulated, presumably as a compensatory mechanism. Neither siPKM1 nor siPK was used as a control because both sequences would be toxic to healthy cells.

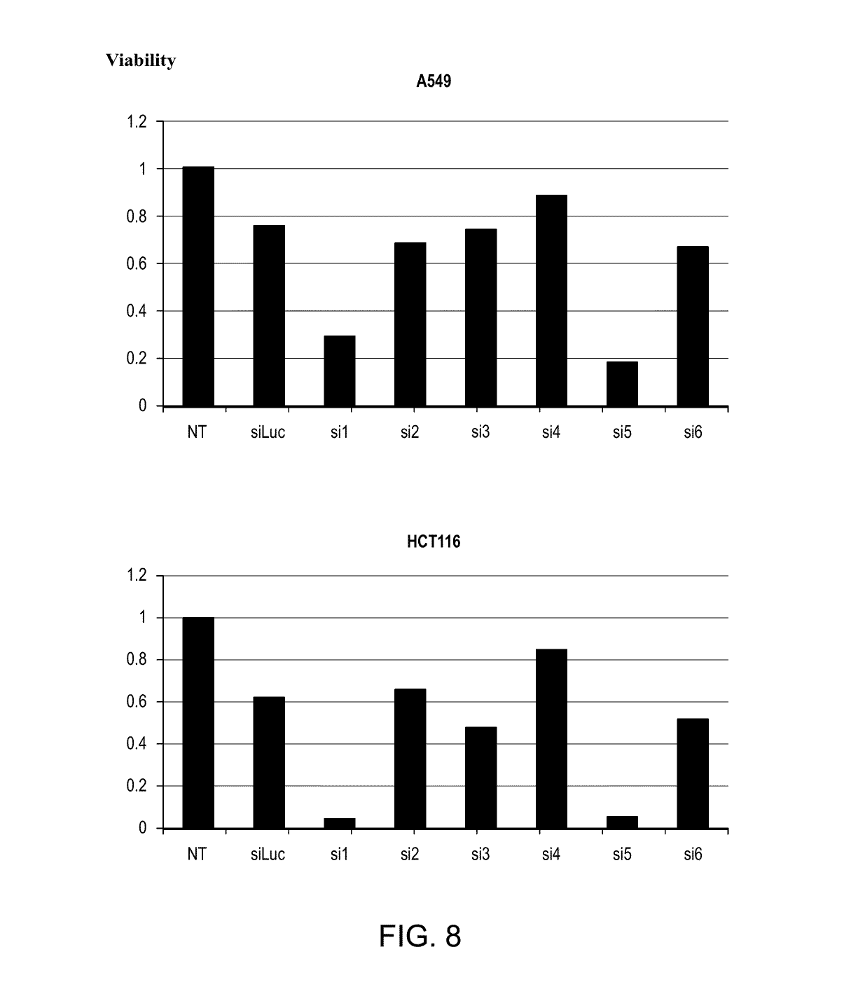

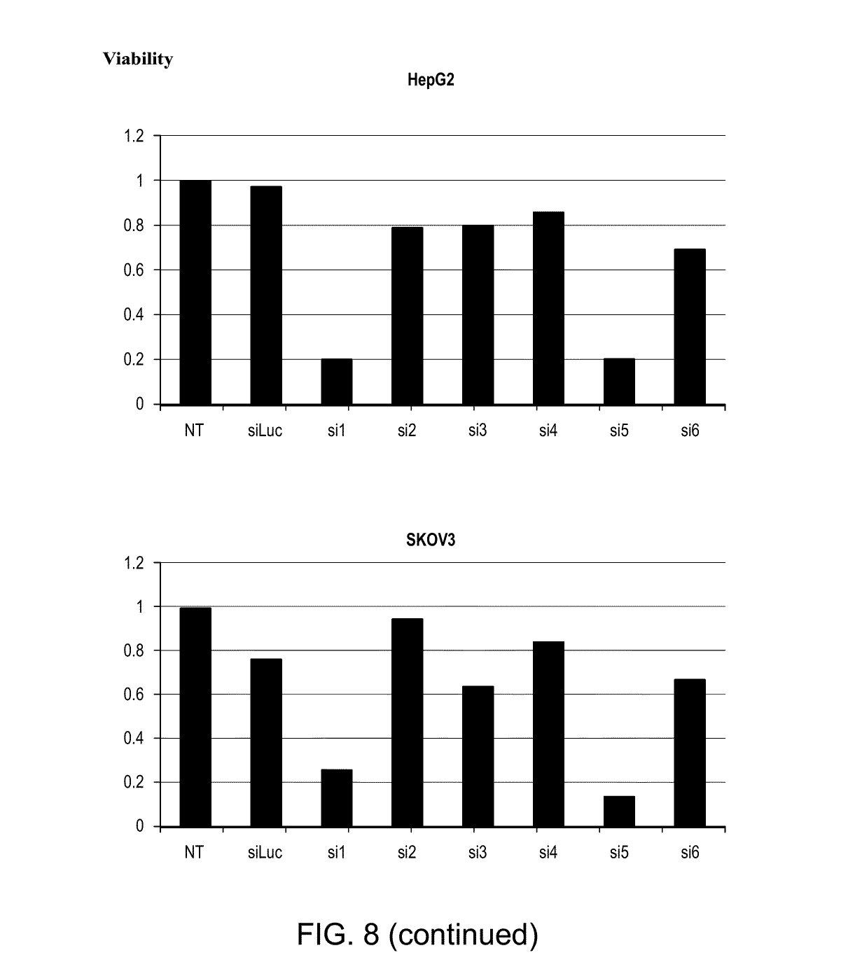

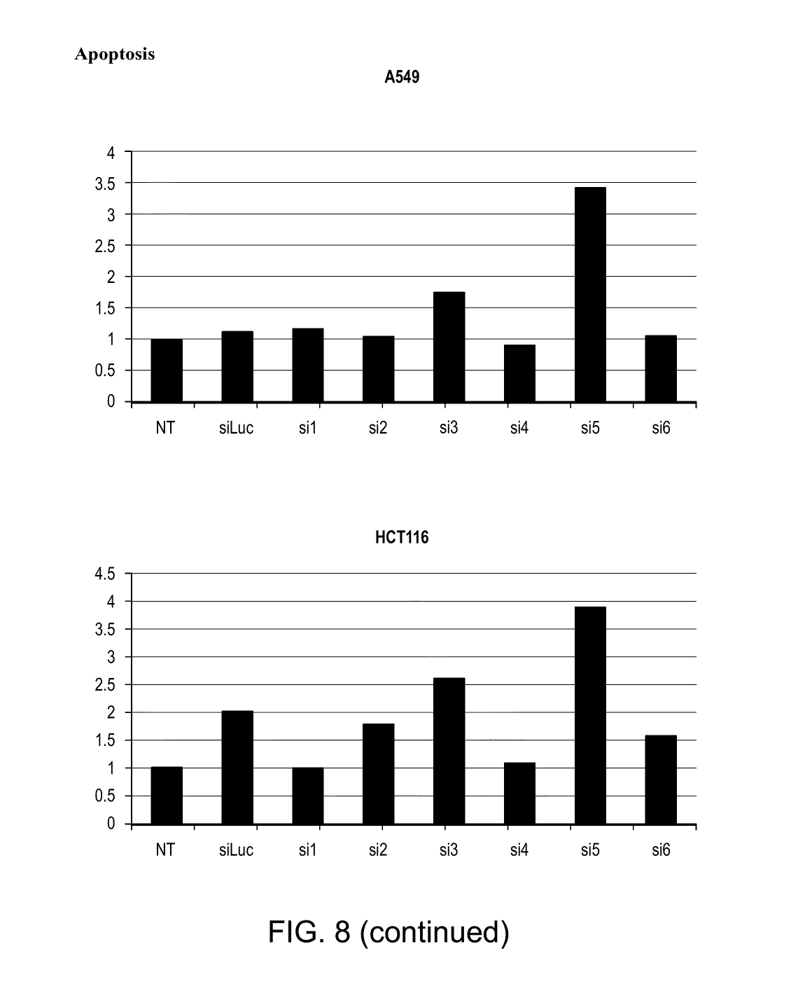

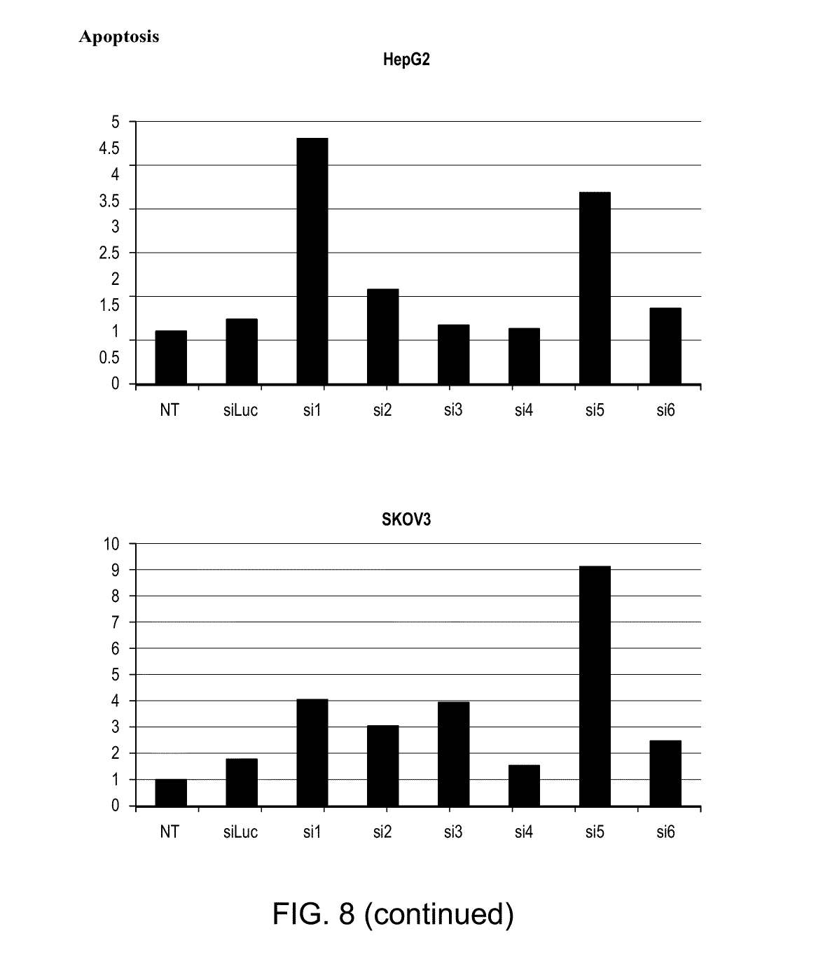

The effect of si1-si6 on viability and apoptosis was investigated in four different cell lines was investigated (FIG. 8). Si5 was observed to have the strongest effect on these two parameters across the four cell lines.

Because of the observed lack of perfect correlation between the ability of a given siRNA to knockdown PKM2 expression and to decrease cell viability, we sought to understand the potential role of off-target effects. Previous work has suggested that 3′ untranslated region (UTR) seed matches, rather than overall identity, are associated with RNAi off-target effects¹⁷. While associated with a negative connotation, “off-target effects” are highly desirable if they result in the translational repression of oncogenes, some of which contain predicted seed matches for the more effective siRNAs. Thus, although the ostensible target of these siRNAs is PKM2, their targets should be thought to include any oncogene whose repression would be therapeutically relevant—with this perspective, they are actually all “on target”. Given the number of potential seed matches for the other siRNAs, confirming off-target effects was not feasible, so this reasoning remains supposition.

### Example 4

### Identification of siRNAs that Confer Specific Knockdown of PKM2

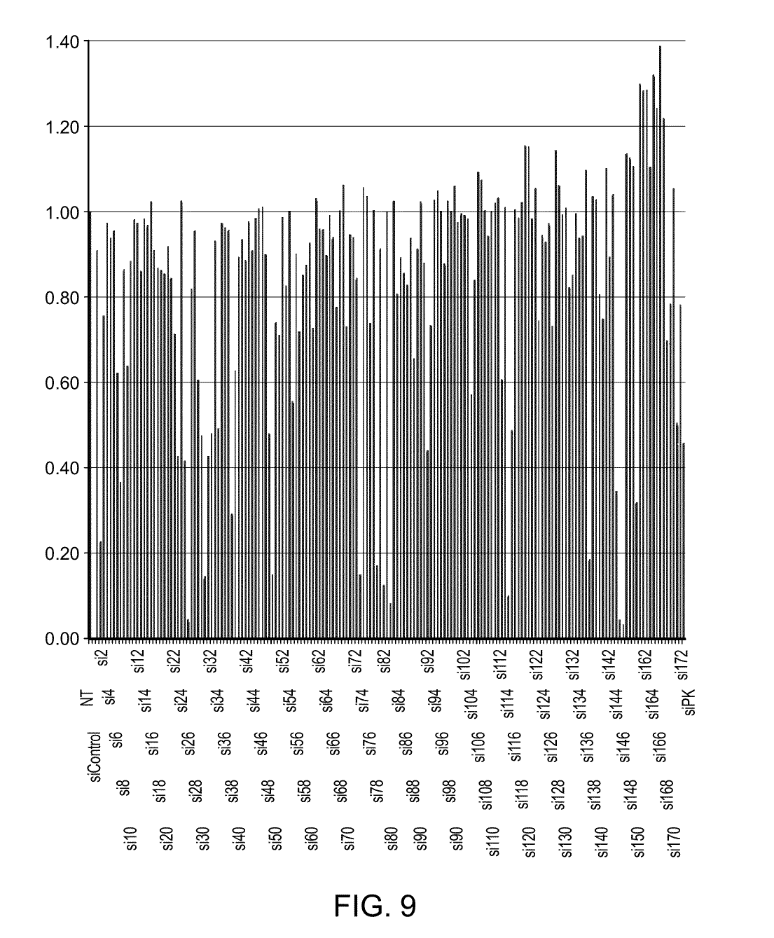

As described in more detail elsewhere herein, we attempted to identify sequences that would target the M2 isoform of pyruvate kinase specifically (FIG. 9).

A custom library of siRNAs that tiled exon 10 (Table 4) was screened. The custom library was synthesized by Thermo Scientific Dharmacon as ONTARGETplus siRNA. The in vivo experiments were conducted using unmodified siRNA. We assessed for the ability of the sequences to decrease cell viability in the human colon cancer cell line HCT116 (FIG. 9). Notably, very few siRNAs (<7%) actually generated a phenotype, as defined by <20% survival relative to untreated cells (Si1, Si27, Si32, Si40, Si52, Si58, Si78, Si83, Si85, Si87, Si122, Si146, Si155, and Si156. See Table 4 for sense sequences). Three sequences from the tiling library—si27, si155, and si156—conferred a particularly-potent inhibition of survival or proliferation (<5% survival): si27, si155, and si156 (see FIG. 9 and Table 4).

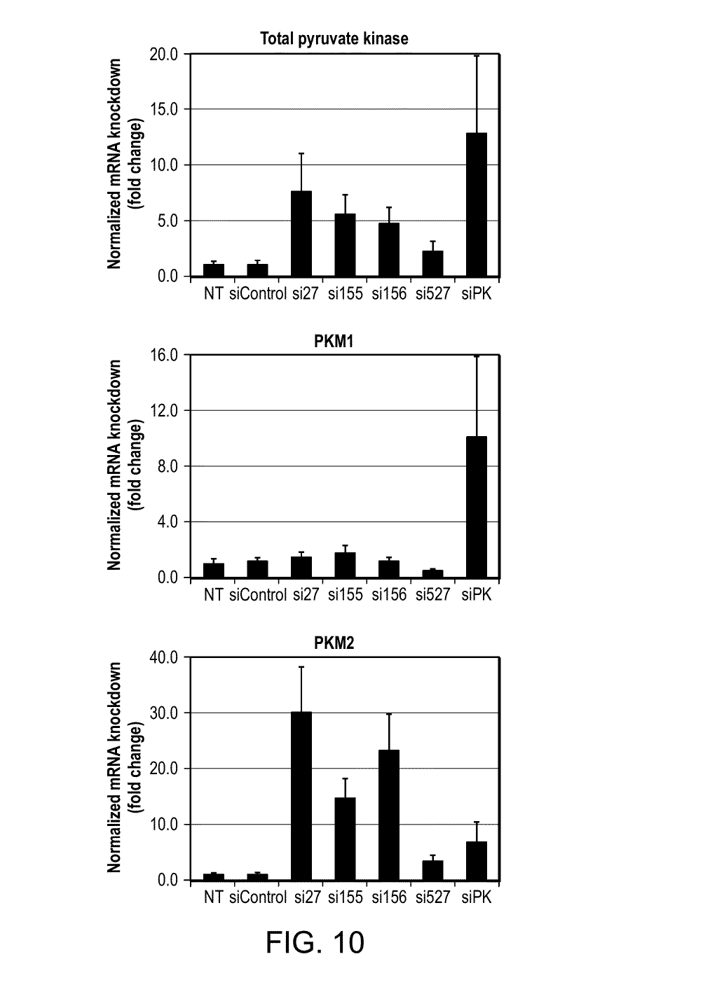

siControl, a sequence that targets firefly luciferase, was used as a negative control. siPK, a commercially-available sequence that is reported to target PKM2, was used as a positive control. However, this siRNA targets sequences that are common to both the M1 and M2 isoforms, as does every commercially available siRNA that targets pyruvate kinase that we have tested. To maximize the specificity of the library, ON-TARGETplus siRNA was used, the only dual strand modification pattern available. Specifically, the sense strand is modified to prevent interaction with RISC and favor antisense strand uptake, and the antisense strand seed region is modified to minimize seed-related off-targeting. To demonstrate that the effects imparted by the most efficacious siRNAs were specific to the knockdown of the M2 isoform, we performed isoform-specific real-time PCR using primers that were designed to amplify either isoform individually or total pyruvate kinase (FIG. 10). The knockdown of PKM2 was both specific and robust for si27, si155, and si156. In contrast, si157, which does not influence cell viability, conferred modest knockdown, and siPK, as expected, resulted in potent knockdown of both PKM1 and PKM2. The pronounced changes at the mRNA level indicate that there is efficient uptake of siRNA.

TABLE 4
Tiling siPKM2 sense strand sequences, representing every possible
19-mer sense strand sequence containing at least one mismatch
between the M1 (exon 9) and M2 (exon 10) isoforms of human
pyruvate kinase. The number of mismatches between the two isoforms
ranges from one (si1, si173) to 15 (si76). Mismatches are
shown in bold. Nucleotides from neighboring exons 8 and 11 are
shown in lowercase. The sequences of the negative control
(siControl) and positive control (siPK, a commercially-
available sequence that targets a region in exon 5, which
is common to both isoforms) are also given. All siRNAs had UU
appended to 3′ termini of the given sense strand sequences.
For example, the siRNA sense strand sequence of the siRNA si1 was
5′-GCGCAUGCAGCACCUGAUUUU-3′ (SEQ ID NO: 250).

| siRNA | Sense Strand Sequence |
|---|---|
| si1 | gcgcaugcagcaccugAU [symbol] (SEQ ID NO: 68) |
| si2 | cgcaugcagcaccugAU [symbol] G (SEQ ID NO: 69) |
| si3 | gcaugcagcaccugAU [symbol] GC (SEQ ID NO: 70) |
| si4 | caugcagcaccugAU [symbol] GC [symbol] (SEQ ID NO: 71) |
| si5 | augcagcaccugAU [symbol] GC [symbol] C (SEQ ID NO: 72) |
| si6 | ugcagcaccugAU [symbol] GC [symbol] CG (SEQ ID NO: 73) |
| si7 | gcagcaccugAU [symbol] GC [symbol] CGU (SEQ ID NO: 74) |
| si8 | cagcaccugAU [symbol] GC [symbol] CGUG (SEQ ID NO: 75) |
| si9 | agcaccugAU [symbol] GC [symbol] CGUGA (SEQ ID NO: 76) |
| si10 | gcaccugAU [symbol] GC [symbol] CGUGAG (SEQ ID NO: 77) |
| si11 | caccugAU [symbol] GC [symbol] CGUGAGG (SEQ ID NO: 78) |
| si12 | accugAU [symbol] GC [symbol] CGUGAGGC (SEQ ID NO: 79) |
| si13 | ccugAU [symbol] GC [symbol] CGUGAGGC [symbol] (SEQ ID NO: 80) |
| si14 | cugAU [symbol] GC [symbol] CGUGAGGC [symbol] G (SEQ ID NO: 81) |
| si15 | ugAU [symbol] GC [symbol] CGUGAGGC [symbol] GA (SEQ ID NO: 82) |
| si16 | gAU [symbol] GC [symbol] CGUGAGGC [symbol] GAG (SEQ ID NO: 83) |
| si17 | AU [symbol] GC [symbol] CGUGAGGC [symbol] GAGG (SEQ ID NO: 84) |
| si18 | U [symbol] GC [symbol] CGUGAGGC [symbol] GAGGC (SEQ ID NO: 85) |
| si19 | [symbol] GC [symbol] CGUGAGGC [symbol] GAGGC [symbol] (SEQ ID NO: 86) |
| si20 | GC [symbol] CGUGAGGC [symbol] GAGGC [symbol] G (SEQ ID NO: 87) |
| si21 | C [symbol] CGUGAGGC [symbol] GAGGC [symbol] GC (SEQ ID NO: 88) |
| si22 | [symbol] CGUGAGGC [symbol] GAGGC [symbol] GCC (SEQ ID NO: 89) |
| si23 | CGUGAGGC [symbol] GAGGC [symbol] GCCA (SEQ ID NO: 90) |
| si24 | GUGAGGC [symbol] GAGGC [symbol] GCCAU (SEQ ID NO: 91) |
| si25 | UGAGGC [symbol] GAGGC [symbol] GCCAU [symbol] (SEQ ID NO: 92) |
| si26 | GAGGC [symbol] GAGGC [symbol] GCCAU [symbol] U (SEQ ID NO: 93) |
| si27 | AGGC [symbol] GAGGC [symbol] GCCAU [symbol] U [symbol] (SEQ ID NO: 94) |
| si28 | GGC [symbol] GAGGC [symbol] GCCAU [symbol] U [symbol] C (SEQ ID NO: 95) |
| si29 | GC [symbol] GAGGC [symbol] GCCAU [symbol] U [symbol] CC (SEQ ID NO: 96) |
| si30 | C [symbol] GAGGC [symbol] GCCAU [symbol] U [symbol] CCA (SEQ ID NO: 97) |
| si31 | [symbol] GAGGC [symbol] GCCAU [symbol] U [symbol] CCAC (SEQ ID NO: 98) |
| si32 | GAGGC [symbol] GCCAU [symbol] U [symbol] CCAC [symbol] (SEQ ID NO: 99) |
| si33 | AGGC [symbol] GCCAU [symbol] U [symbol] CCAC [symbol] [symbol] (SEQ ID NO: 100) |
| si34 | GGC [symbol] GCCAU [symbol] U [symbol] CCAC [symbol] [symbol] [symbol] (SEQ ID NO: 101) |
| si35 | GC [symbol] GCCAU [symbol] U [symbol] CCAC [symbol] [symbol] [symbol] [symbol] (SEQ ID NO: 102) |
| si36 | C [symbol] GCCAU [symbol] U [symbol] CCAC [symbol] [symbol] [symbol] [symbol] A (SEQ ID NO: 103) |
| si37 | [symbol] GCCAU [symbol] U [symbol] CCAC [symbol] [symbol] [symbol] [symbol] A [symbol] (SEQ ID NO: 104) |
| si38 | GCCAU [symbol] U [symbol] CCAC [symbol] [symbol] [symbol] [symbol] A [symbol] U (SEQ ID NO: 105) |
| si39 | CCAU [symbol] U [symbol] CCAC [symbol] [symbol] [symbol] [symbol] A [symbol] UU (SEQ ID NO: 106) |
| si40 | CAU [symbol] U [symbol] CCAC [symbol] [symbol] [symbol] [symbol] A [symbol] UU [symbol] (SEQ ID NO: 107) |
| si41 | AU [symbol] U [symbol] CCAC [symbol] [symbol] [symbol] [symbol] A [symbol] UU [symbol] U (SEQ ID NO: 108) |
| si42 | U [symbol] U [symbol] CCAC [symbol] [symbol] [symbol] [symbol] A [symbol] UU [symbol] UU (SEQ ID NO: 109) |
| si43 | [symbol] U [symbol] CCAC [symbol] [symbol] [symbol] [symbol] A [symbol] UU [symbol] UUU (SEQ ID NO: 110) |
| si44 | U [symbol] CCAC [symbol] [symbol] [symbol] [symbol] A [symbol] UU [symbol] UUUG (SEQ ID NO: 111) |
| si45 | [symbol] CCAC [symbol] [symbol] [symbol] [symbol] A [symbol] UU [symbol] UUUGA (SEQ ID NO: 112) |
| si46 | CCAC [symbol] [symbol] [symbol] [symbol] A [symbol] UU [symbol] UUUGA [symbol] (SEQ ID NO: 113) |
| si47 | CAC [symbol] [symbol] [symbol] [symbol] A [symbol] UU [symbol] UUUGA [symbol] G (SEQ ID NO: 114) |
| si48 | AC [symbol] [symbol] [symbol] [symbol] A [symbol] [symbol] U [symbol] UUUGA [symbol] GA (SEQ ID NO: 115) |
| si49 | C [symbol] [symbol] [symbol] [symbol] A [symbol] [symbol] U [symbol] UUUGA [symbol] GAA (SEQ ID NO: 116) |
| si50 | [symbol] [symbol] [symbol] [symbol] A [symbol] [symbol] U [symbol] [symbol] UUUGA [symbol] GAAC (SEQ ID NO: 117) |
| si51 | [symbol] [symbol] [symbol] A [symbol] [symbol] [symbol] U [symbol] UUUGA [symbol] GAACU (SEQ ID NO: 118) |
| si52 | [symbol] [symbol] A [symbol] [symbol] U [symbol] UUUGA [symbol] GAACU [symbol] (SEQ ID NO: 119) |
| si53 | [symbol] A [symbol] [symbol] U [symbol] UUUGA [symbol] GAACU [symbol] [symbol] (SEQ ID NO: 120) |
| si54 | A [symbol] [symbol] U [symbol] UUUGA [symbol] GAACU [symbol] [symbol] [symbol] (SEQ ID NO: 121) |
| si55 | [symbol] [symbol] U [symbol] UUUGA [symbol] GAACU [symbol] [symbol] [symbol] [symbol] (SEQ ID NO: 122) |
| si56 | [symbol] U [symbol] UUUGA [symbol] GAACU [symbol] [symbol] [symbol] [symbol] C (SEQ ID NO: 123) |
| si57 | U [symbol] UUUGA [symbol] GAACU [symbol] [symbol] [symbol] [symbol] CG (SEQ ID NO: 124) |
| si58 | [symbol] UUUGA [symbol] GAACU [symbol] [symbol] [symbol] [symbol] CG [symbol] (SEQ ID NO: 125) |
| si59 | UUUGA [symbol] GAACU [symbol] [symbol] [symbol] [symbol] CG [symbol] [symbol] (SEQ ID NO: 126) |
| si60 | UUGA [symbol] GAACU [symbol] [symbol] [symbol] [symbol] CG [symbol] [symbol] [symbol] (SEQ ID NO: 127) |
| si61 | UGA [symbol] GAACU [symbol] [symbol] [symbol] [symbol] CG [symbol] [symbol] [symbol] [symbol] (SEQ ID NO: 128) |
| si62 | GA [symbol] GAACU [symbol] [symbol] [symbol] [symbol] CG [symbol] [symbol] [symbol] [symbol] [symbol] (SEQ ID NO: 129) |
| si63 | A [symbol] GAACU [symbol] [symbol] [symbol] [symbol] CG [symbol] [symbol] [symbol] [symbol] [symbol] C (SEQ ID NO: 130) |
| si64 | [symbol] GAACU [symbol] [symbol] [symbol] [symbol] CG [symbol] [symbol] [symbol] [symbol] [symbol] C [symbol] (SEQ ID NO: 131) |
| si65 | GAACU [symbol] [symbol] [symbol] [symbol] CG [symbol] [symbol] [symbol] [symbol] [symbol] C [symbol] [symbol] (SEQ ID NO: 132) |
| si66 | AACU [symbol] [symbol] [symbol] [symbol] CG [symbol] [symbol] [symbol] [symbol] [symbol] C [symbol] [symbol] [symbol] (SEQ ID NO: 133) |
| si67 | ACU [symbol] [symbol] [symbol] [symbol] CG [symbol] [symbol] [symbol] [symbol] [symbol] C [symbol] [symbol] [symbol] [symbol] (SEQ ID NO: 134) |
| si68 | CU [symbol] [symbol] [symbol] [symbol] CG [symbol] [symbol] [symbol] [symbol] [symbol] C [symbol] [symbol] [symbol] [symbol] [symbol] (SEQ ID NO: 135) |
| si69 | U [symbol] [symbol] [symbol] [symbol] CG [symbol] [symbol] [symbol] [symbol] [symbol] C [symbol] [symbol] [symbol] [symbol] [symbol] [symbol] (SEQ ID NO: 136) |
| si70 | [symbol] [symbol] [symbol] [symbol] CG [symbol] [symbol] [symbol] [symbol] [symbol] C [symbol] [symbol] [symbol] [symbol] [symbol] [symbol] [symbol] (SEQ ID NO: 137) |
| si71 | [symbol] [symbol] [symbol] CG [symbol] [symbol] [symbol] [symbol] [symbol] C [symbol] [symbol] [symbol] [symbol] [symbol] [symbol] [symbol] [symbol] (SEQ ID NO: 138) |
| si72 | [symbol] [symbol] CG [symbol] [symbol] [symbol] [symbol] [symbol] C [symbol] [symbol] [symbol] [symbol] [symbol] [symbol] [symbol] [symbol] C (SEQ ID NO: 139) |
| si73 | [symbol] CG [symbol] [symbol] [symbol] [symbol] [symbol] C [symbol] [symbol] [symbol] [symbol] [symbol] [symbol] [symbol] [symbol] CC (SEQ ID NO: 140) |
| si74 | CG [symbol] [symbol] [symbol] [symbol] [symbol] C [symbol] [symbol] [symbol] [symbol] [symbol] [symbol] [symbol] [symbol] CCA (SEQ ID NO: 141) |
| si75 | G [symbol] [symbol] [symbol] [symbol] [symbol] C [symbol] [symbol] [symbol] [symbol] [symbol] [symbol] [symbol] [symbol] CCA [symbol] (SEQ ID NO: 142) |
| si76 | [symbol] [symbol] [symbol] [symbol] [symbol] C [symbol] [symbol] [symbol] [symbol] [symbol] [symbol] [symbol] [symbol] CCA [symbol] [symbol] (SEQ ID NO: 143) |
| si77 | [symbol] [symbol] [symbol] [symbol] C [symbol] [symbol] [symbol] [symbol] [symbol] [symbol] [symbol] [symbol] CCA [symbol] [symbol] G (SEQ ID NO: 144) |
| si78 | [symbol] [symbol] [symbol] C [symbol] [symbol] [symbol] [symbol] [symbol] [symbol] [symbol] [symbol] CCA [symbol] [symbol] GA (SEQ ID NO: 145) |
| si79 | [symbol] [symbol] C [symbol] [symbol] [symbol] [symbol] [symbol] [symbol] [symbol] [symbol] CCA [symbol] [symbol] GAC (SEQ ID NO: 146) |
| si80 | [symbol] C [symbol] [symbol] [symbol] [symbol] [symbol] [symbol] [symbol] [symbol] CCA [symbol] [symbol] GACC (SEQ ID NO: 147) |
| si81 | C [symbol] [symbol] [symbol] [symbol] [symbol] [symbol] [symbol] [symbol] CCA [symbol] [symbol] GACC [symbol] (SEQ ID NO: 148) |
| si82 | [symbol] [symbol] [symbol] [symbol] [symbol] [symbol] [symbol] [symbol] CCA [symbol] [symbol] GACC [symbol] C (SEQ ID NO: 149) |
| si83 | [symbol] [symbol] [symbol] [symbol] [symbol] [symbol] [symbol] CCA [symbol] [symbol] GACC [symbol] CA [symbol] A (SEQ ID NO: 150) |
| si84 | [symbol] [symbol] [symbol] [symbol] [symbol] [symbol] CCA [symbol] [symbol] GACC [symbol] CA [symbol] (SEQ ID NO: 151) |
| si85 | [symbol] [symbol] [symbol] [symbol] [symbol] CCA [symbol] [symbol] GACC [symbol] CA [symbol] [symbol] (SEQ ID NO: 152) |
| si86 | [symbol] [symbol] [symbol] [symbol] CCA [symbol] [symbol] GACC [symbol] CA [symbol] [symbol] G (SEQ ID NO: 153) |
| si87 | [symbol] [symbol] [symbol] CCA [symbol] [symbol] GACC [symbol] CA [symbol] [symbol] GA (SEQ ID NO: 154) |
| si88 | [symbol] [symbol] CCA [symbol] [symbol] GACC [symbol] CA [symbol] [symbol] GAA (SEQ ID NO: 155) |
| si89 | [symbol] CCA [symbol] [symbol] GACC [symbol] CA [symbol] [symbol] GAAG (SEQ ID NO: 156) |
| si90 | CCA [symbol] [symbol] GACC [symbol] CA [symbol] [symbol] GAAGC (SEQ ID NO: 157) |
| si91 | CA [symbol] [symbol] GACC [symbol] CA [symbol] [symbol] GAAGCC (SEQ ID NO: 158) |
| si92 | A [symbol] [symbol] GACC [symbol] CA [symbol] [symbol] GAAGCCA (SEQ ID NO: 159) |
| si93 | [symbol] [symbol] GACC [symbol] CA [symbol] [symbol] GAAGCCA [symbol] (SEQ ID NO: 160) |
| si94 | [symbol] GACC [symbol] CA [symbol] [symbol] GAAGCCA [symbol] [symbol] (SEQ ID NO: 161) |
| si95 | GACC [symbol] CA [symbol] [symbol] GAAGCCA [symbol] [symbol] G (SEQ ID NO: 162) |
| si96 | ACC [symbol] CA [symbol] [symbol] GAAGCCA [symbol] [symbol] GC (SEQ ID NO: 163) |
| si97 | CC [symbol] CA [symbol] [symbol] GAAGCCA [symbol] [symbol] GCC (SEQ ID NO: 164) |
| si98 | C [symbol] CA [symbol] [symbol] GAAGCCA [symbol] [symbol] GCC [symbol] (SEQ ID NO: 165) |
| si99 | [symbol] CA [symbol] [symbol] GAAGCCA [symbol] [symbol] GCC [symbol] U (SEQ ID NO: 166) |
| si100 | CA [symbol] [symbol] GAAGCCA [symbol] [symbol] GCC [symbol] UG (SEQ ID NO: 167) |
| si101 | A [symbol] [symbol] GAAGCCA [symbol] [symbol] GCC [symbol] UGG (SEQ ID NO: 168) |
| si102 | [symbol] [symbol] GAAGCCA [symbol] [symbol] GCC [symbol] UGGG (SEQ ID NO: 169) |
| si103 | [symbol] GAAGCCA [symbol] [symbol] GCC [symbol] UGGG [symbol] (SEQ ID NO: 170) |
| si104 | GAAGCCA [symbol] [symbol] GCC [symbol] UGGG [symbol] [symbol] (SEQ ID NO: 171) |
| si105 | AAGCCA [symbol] [symbol] GCC [symbol] UGGG [symbol] [symbol] [symbol] (SEQ ID NO: 172) |
| si106 | AGCCA [symbol] [symbol] GCC [symbol] UGGG [symbol] [symbol] [symbol] C (SEQ ID NO: 173) |
| si107 | GCCA [symbol] [symbol] GCC [symbol] UGGG [symbol] [symbol] [symbol] CG (SEQ ID NO: 174) |
| si108 | CCA [symbol] [symbol] GCC [symbol] UGGG [symbol] [symbol] [symbol] CGU (SEQ ID NO: 175) |
| si109 | CA [symbol] [symbol] GCC [symbol] UGGG [symbol] [symbol] [symbol] CGUG (SEQ ID NO: 176) |
| si110 | A [symbol] [symbol] GCC [symbol] UGGG [symbol] [symbol] [symbol] CGUGG (SEQ ID NO: 177) |
| si111 | [symbol] [symbol] GCC [symbol] UGGG [symbol] [symbol] [symbol] CGUGGA (SEQ ID NO: 178) |
| si112 | [symbol] GCC [symbol] UGGG [symbol] [symbol] [symbol] CGUGGAG (SEQ ID NO: 179) |
| si113 | GCC [symbol] UGGG [symbol] [symbol] [symbol] CGUGGAGG (SEQ ID NO: 180) |
| si114 | CC [symbol] UGGG [symbol] [symbol] [symbol] CGUGGAGGC (SEQ ID NO: 181) |
| si115 | C [symbol] UGGG [symbol] [symbol] [symbol] CGUGGAGGC [symbol] (SEQ ID NO: 182) |
| si116 | [symbol] UGGG [symbol] [symbol] [symbol] CGUGGAGGC [symbol] U (SEQ ID NO: 183) |
| si117 | UGGG [symbol] [symbol] [symbol] CGUGGAGGC [symbol] UC (SEQ ID NO: 184) |
| si118 | GGG [symbol] [symbol] [symbol] CGUGGAGGC [symbol] UC [symbol] (SEQ ID NO: 185) |
| si119 | GG [symbol] [symbol] [symbol] CGUGGAGGC [symbol] UC [symbol] U (SEQ ID NO: 186) |
| si120 | G [symbol] [symbol] [symbol] CGUGGAGGC [symbol] UC [symbol] U [symbol] (SEQ ID NO: 187) |
| si121 | [symbol] [symbol] [symbol] CGUGGAGGC [symbol] UC [symbol] U [symbol] [symbol] (SEQ ID NO: 188) |
| si122 | [symbol] [symbol] CGUGGAGGC [symbol] UC [symbol] U [symbol] [symbol] A (SEQ ID NO: 189) |
| si123 | [symbol] CGUGGAGGC [symbol] UC [symbol] U [symbol] [symbol] AA (SEQ ID NO: 190) |
| si124 | CGUGGAGGC [symbol] UC [symbol] U [symbol] [symbol] AAG (SEQ ID NO: 191) |
| si125 | GUGGAGGC [symbol] UC [symbol] U [symbol] [symbol] AAGU (SEQ ID NO: 192) |
| si126 | UGGAGGC [symbol] UC [symbol] U [symbol] [symbol] AAGUG (SEQ ID NO: 193) |
| si127 | GGAGGC [symbol] UC [symbol] U [symbol] [symbol] AAGUG [symbol] (SEQ ID NO: 194) |
| si128 | GAGGC [symbol] UC [symbol] U [symbol] [symbol] AAGUG [symbol] U (SEQ ID NO: 195) |
| si129 | AGGC [symbol] UC [symbol] U [symbol] [symbol] AAGUG [symbol] U [symbol] (SEQ ID NO: 196) |
| si130 | GGC [symbol] UC [symbol] U [symbol] [symbol] AAGUG [symbol] U [symbol] [symbol] (SEQ ID NO: 197) |
| si131 | GC [symbol] UC [symbol] U [symbol] [symbol] AAGUG [symbol] U [symbol] [symbol] [symbol] (SEQ ID NO: 198) |
| si132 | C [symbol] UC [symbol] U [symbol] [symbol] AAGUG [symbol] U [symbol] [symbol] [symbol] [symbol] (SEQ ID NO: 199) |
| si133 | [symbol] UC [symbol] U [symbol] [symbol] AAGUG [symbol] U [symbol] [symbol] [symbol] [symbol] [symbol] (SEQ ID NO: 200) |
| si134 | UC [symbol] U [symbol] [symbol] AAGUG [symbol] U [symbol] [symbol] [symbol] [symbol] [symbol] G (SEQ ID NO: 201) |
| si135 | C [symbol] U [symbol] [symbol] AAGUG [symbol] U [symbol] [symbol] [symbol] [symbol] [symbol] GG (SEQ ID NO: 202) |
| si136 | [symbol] U [symbol] [symbol] AAGUG [symbol] U [symbol] [symbol] [symbol] [symbol] [symbol] GGG (SEQ ID NO: 203) |
| si137 | U [symbol] [symbol] AAGUG [symbol] U [symbol] [symbol] [symbol] [symbol] [symbol] GGGG (SEQ ID NO: 204) |
| si138 | [symbol] [symbol] AAGUG [symbol] U [symbol] [symbol] [symbol] [symbol] [symbol] GGGGC (SEQ ID NO: 205) |
| si139 | [symbol] AAGUG [symbol] U [symbol] [symbol] [symbol] [symbol] [symbol] GGGGC [symbol] (SEQ ID NO: 206) |
| si140 | AAGUG [symbol] U [symbol] [symbol] [symbol] [symbol] [symbol] GGGGC [symbol] [symbol] (SEQ ID NO: 207) |
| si141 | AGUG [symbol] U [symbol] [symbol] [symbol] [symbol] [symbol] GGGGC [symbol] [symbol] U (SEQ ID NO: 208) |
| si142 | GUG [symbol] U [symbol] [symbol] [symbol] [symbol] [symbol] GGGGC [symbol] [symbol] U [symbol] (SEQ ID NO: 209) |
| si143 | UG [symbol] U [symbol] [symbol] [symbol] [symbol] [symbol] GGGGC [symbol] [symbol] U [symbol] A (SEQ ID NO: 210) |
| si144 | G [symbol] U [symbol] [symbol] [symbol] [symbol] [symbol] GGGGC [symbol] [symbol] U [symbol] AU (SEQ ID NO: 211) |
| si145 | [symbol] U [symbol] [symbol] [symbol] [symbol] [symbol] GGGGC [symbol] [symbol] U [symbol] AU [symbol] (SEQ ID NO: 212) |
| si146 | U [symbol] [symbol] [symbol] [symbol] [symbol] GGGGC [symbol] [symbol] U [symbol] AU [symbol] G (SEQ ID NO: 213) |
| si147 | [symbol] [symbol] [symbol] [symbol] [symbol] GGGGC [symbol] [symbol] U [symbol] AU [symbol] GU (SEQ ID NO: 214) |
| si148 | [symbol] [symbol] [symbol] [symbol] GGGGC [symbol] [symbol] U [symbol] AU [symbol] GU [symbol] (SEQ ID NO: 215) |
| si149 | [symbol] [symbol] [symbol] GGGGC [symbol] [symbol] U [symbol] AU [symbol] GU [symbol] C (SEQ ID NO: 216) |
| si150 | [symbol] [symbol] GGGGC [symbol] [symbol] U [symbol] AU [symbol] GU [symbol] CU (SEQ ID NO: 217) |
| si151 | [symbol] GGGGC [symbol] [symbol] U [symbol] AU [symbol] GU [symbol] CU [symbol] (SEQ ID NO: 218) |
| si152 | GGGGC [symbol] [symbol] U [symbol] AU [symbol] GU [symbol] CU [symbol] A (SEQ ID NO: 219) |
| si153 | GGGC [symbol] [symbol] U [symbol] AU [symbol] GU [symbol] CU [symbol] AC (SEQ ID NO: 220) |
| si154 | GGC [symbol] [symbol] U [symbol] AU [symbol] GU [symbol] CU [symbol] AC [symbol] (SEQ ID NO: 221) |
| si155 | GC [symbol] [symbol] U [symbol] AU [symbol] GU [symbol] CU [symbol] AC [symbol] [symbol] (SEQ ID NO: 222) |
| si156 | C [symbol] [symbol] U [symbol] AU [symbol] GU [symbol] CU [symbol] AC [symbol] [symbol] A (SEQ ID NO: 223) |
| si157 | [symbol] [symbol] U [symbol] AU [symbol] GU [symbol] CU [symbol] AC [symbol] [symbol] AG (SEQ ID NO: 224) |
| si158 | [symbol] U [symbol] AU [symbol] GU [symbol] CU [symbol] AC [symbol] [symbol] AGU (SEQ ID NO: 225) |
| si159 | U [symbol] AU [symbol] GU [symbol] CU [symbol] AC [symbol] [symbol] AGUC (SEQ ID NO: 226) |
| si160 | [symbol] AU [symbol] GU [symbol] CU [symbol] AC [symbol] [symbol] AGUCU (SEQ ID NO: 227) |
| si161 | AU [symbol] GU [symbol] CU [symbol] AC [symbol] [symbol] AGUCUG (SEQ ID NO: 228) |
| si162 | U [symbol] GU [symbol] CU [symbol] AC [symbol] [symbol] AGUCUGG (SEQ ID NO: 229) |
| si163 | [symbol] GU [symbol] CU [symbol] AC [symbol] [symbol] AGUCUGGC (SEQ ID NO: 230) |
| si164 | GU [symbol] CU [symbol] AC [symbol] [symbol] AGUCUGGCA (SEQ ID NO: 231) |
| si165 | U [symbol] CU [symbol] AC [symbol] [symbol] AGUCUGGCAG (SEQ ID NO: 232) |
| si166 | [symbol] CU [symbol] AC [symbol] [symbol] AGUCUGGCAGg (SEQ ID NO: 233) |
| si167 | CU [symbol] AC [symbol] [symbol] AGUCUGGCAGgu (SEQ ID NO: 234) |
| si168 | U [symbol] AC [symbol] [symbol] AGUCUGGCAGguc (SEQ ID NO: 235) |
| si169 | [symbol] AC [symbol] [symbol] AGUCUGGCAGgucu (SEQ ID NO: 236) |
| si170 | AC [symbol] [symbol] AGUCUGGCAGgucug (SEQ ID NO: 237) |
| si171 | C [symbol] [symbol] AGUCUGGCAGgucugc (SEQ ID NO: 238) |
| si172 | [symbol] [symbol] AGUCUGGCAGgucugcu (SEQ ID NO: 239) |
| si173 | [symbol] AGUCUGGCAGgucugcuc (SEQ ID NO: 240) |
| siControl | CUUACGCUGAGUACUUCGA (SEQ ID NO: 241) |
| siPK | GGACCUGAGAUCCGAACUG (SEQ ID NO: 242) |

### Example 5

### siRNAs that Potently Knock Down PKM2 Reduce Cell Viability and Induce Apoptosis

The target sequences of siRNAs determined to show potent knockdown in HCT116 (FIG. 9) are given below:

| (SEQ ID NO: 1) | |
|---|---|
| Si1 | GCGCAUGCAGCACCUGAUUUU |
| (SEQ ID NO: 2) | |
| Si27 | AGGCAGAGGCUGCCAUCUAUU |
| (SEQ ID NO: 3) | |
| Si32 | GAGGCUGCCAUCUACCACUUU |
| (SEQ ID NO: 4) | |
| Si40 | CAUCUACCACUUGCAAUUAUU |
| (SEQ ID NO: 5) | |
| Si52 | GCAAUUAUUUGAGGAACUCUU |
| (SEQ ID NO: 6) | |
| Si58 | AUUUGAGGAACUCCGCCGCUU |
| (SEQ ID NO: 7) | |
| Si78 | UGGCGCCCAUUACCAGCGAUU |
| (SEQ ID NO: 8) | |
| Si83 | CCCAUUACCAGCGACCCCAUU |
| (SEQ ID NO: 9) | |
| Si85 | CAUUACCAGCGACCCCACAUU |
| (SEQ ID NO: 10) | |
| Si87 | UUACCAGCGACCCCACAGAUU |
| (SEQ ID NO: 11) | |
| Si122 | GCCGUGGAGGCCUCCUUCAUU |
| (SEQ ID NO: 12) | |
| Si146 | UGCAGUGGGGCCAUAAUCGUU |
| (SEQ ID NO: 13) | |
| Si155 | GCCAUAAUCGUCCUCACCAUU |
| (SEQ ID NO: 14) | |
| Si156 | CCAUAAUCGUCCUCACCAAUU |

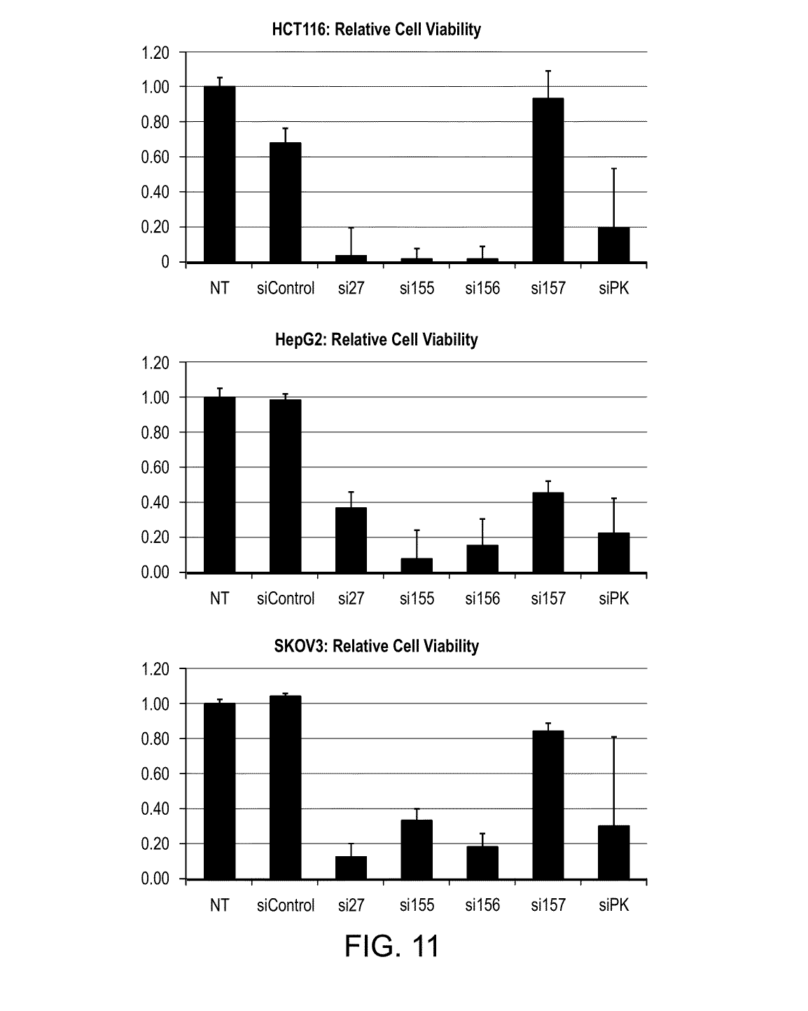

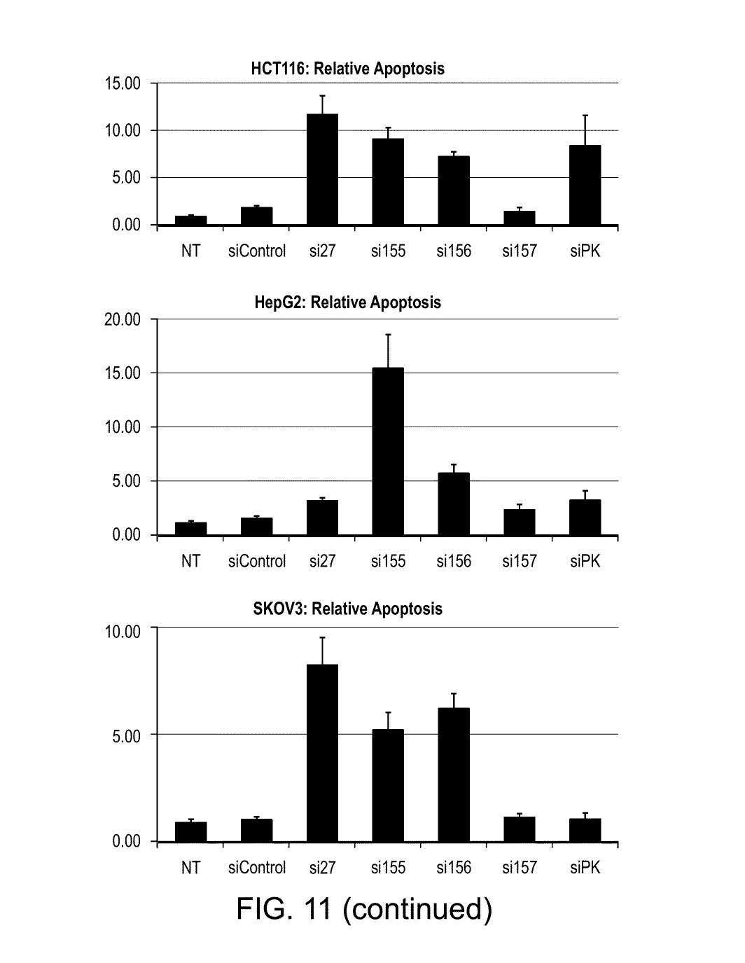

The effect of the three most active siRNAs (si27, si155, and si156) on viability and apoptosis was examined in HCT116, HepG2, and SKOV3 cells (FIG. 11). HepG2, a hepatocellular carcinoma cell line, and SKOV3, an ovarian carcinoma cell line, were selected to complement HCT116, in which the original screen had been performed, because the current most advanced siRNA delivery systems successfully delivers RNAi therapeutics to the liver^{12, 25} and ovaries¹³ in vivo.

The CellTiterGlo® Assay yields a luminescent signal that is proportional to the amount of ATP present. To measure viable cells, a fluorogenic, cell-permeant peptide substrate (GF-AFC) was used. Using the Apotox-Glo™ Triplex Assay, we measured both viability and Caspase3/7-mediated apoptosis in all three cell lines. Though their relative efficacy varies slightly between cell lines, si27, si155, and si156 consistently reduce cell viability and increase apoptosis in each cancer line examined, whereas the controls do not. Importantly, these data confirm that the decreased relative viability is attributable to programmed cell death rather than simply to inhibition of proliferation.

Progressing toward in vivo studies, we elected to focus on si156, because it was among the most active in all cell lines tested. To show that si156 was active in many cancers other than colon cancer, four cell lines (HepG2, SKOV3, A549, and KB), representing four different cancer types (liver, ovarian, lung, and nasopharyngeal, respectively) were transfected with either siControl or si156

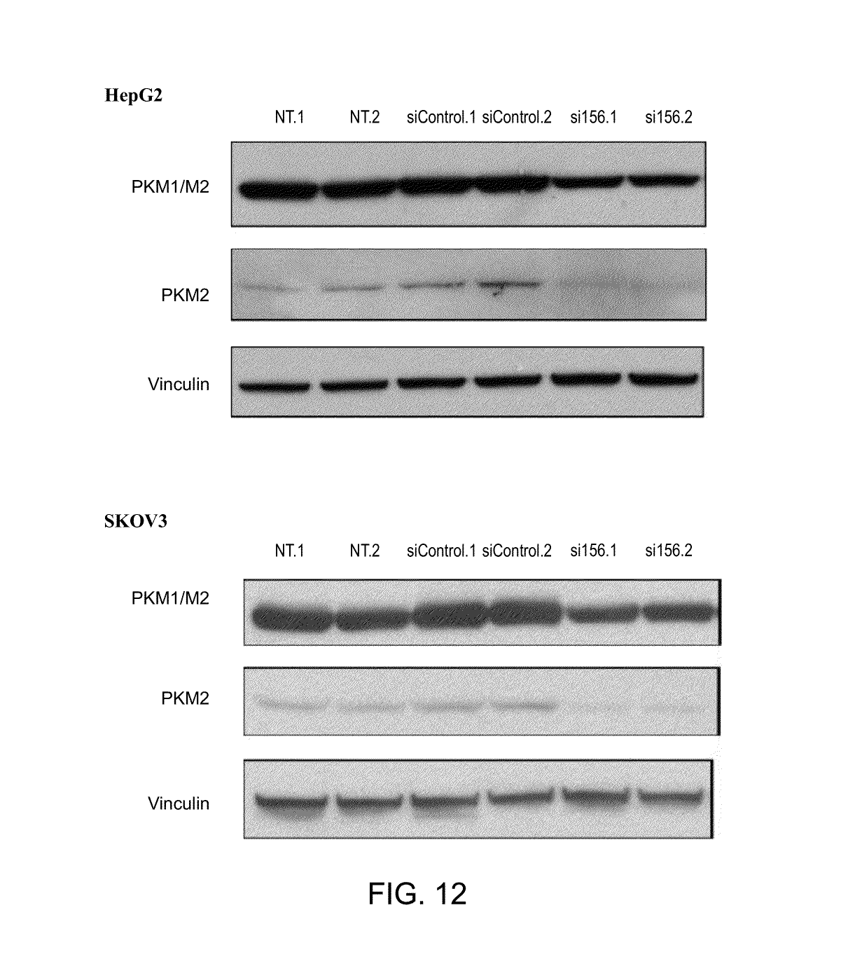

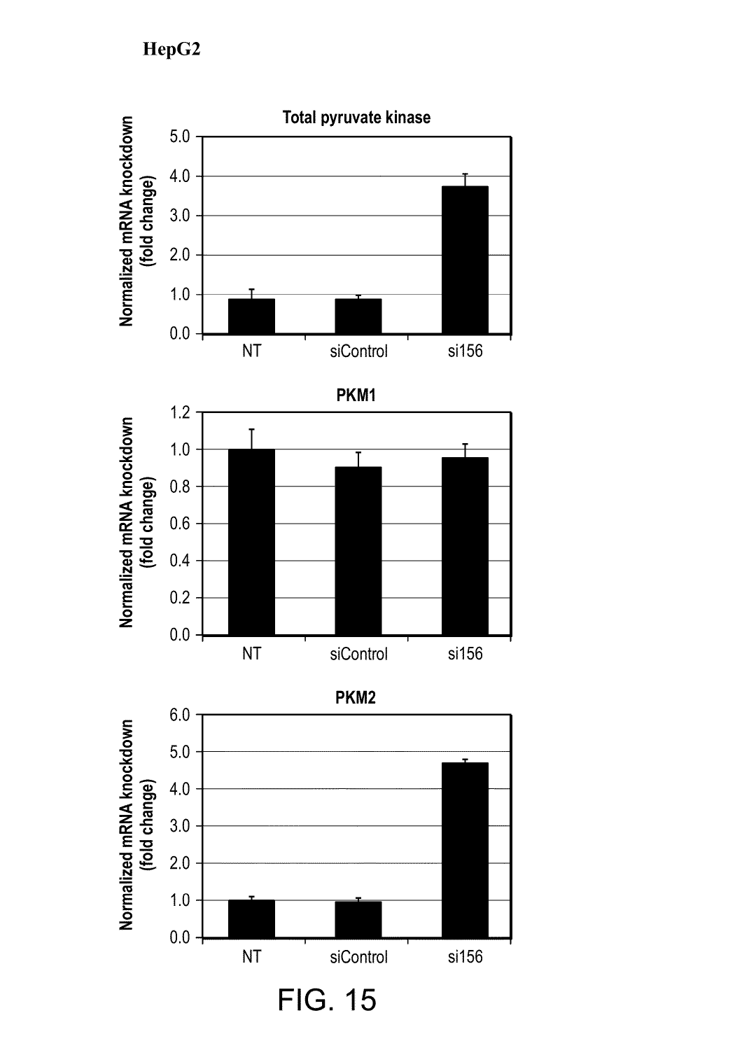

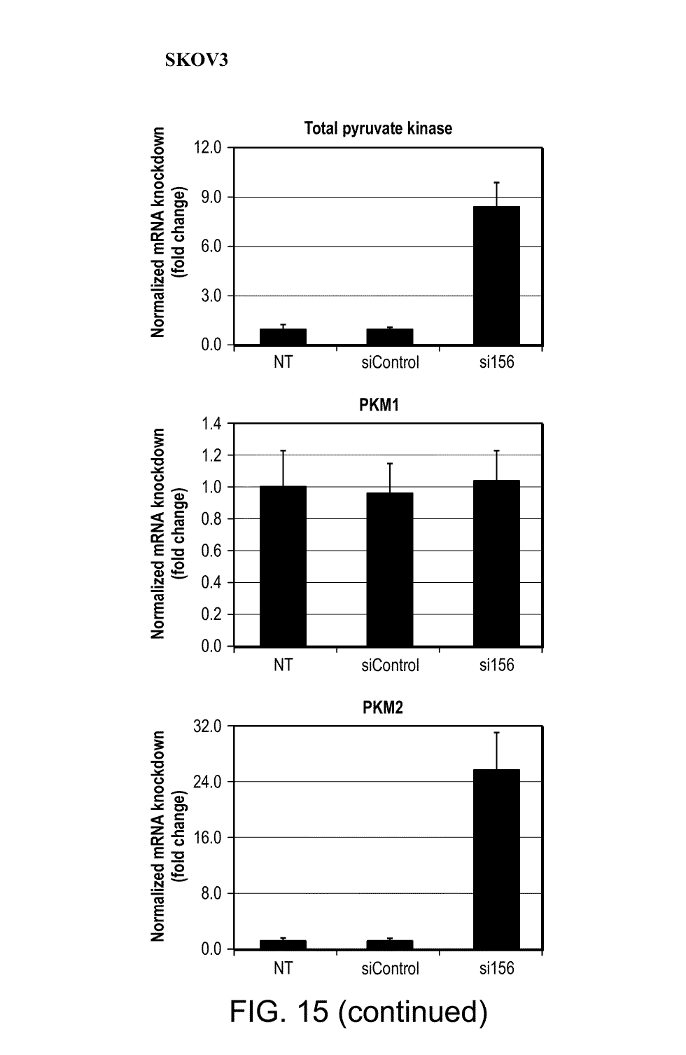

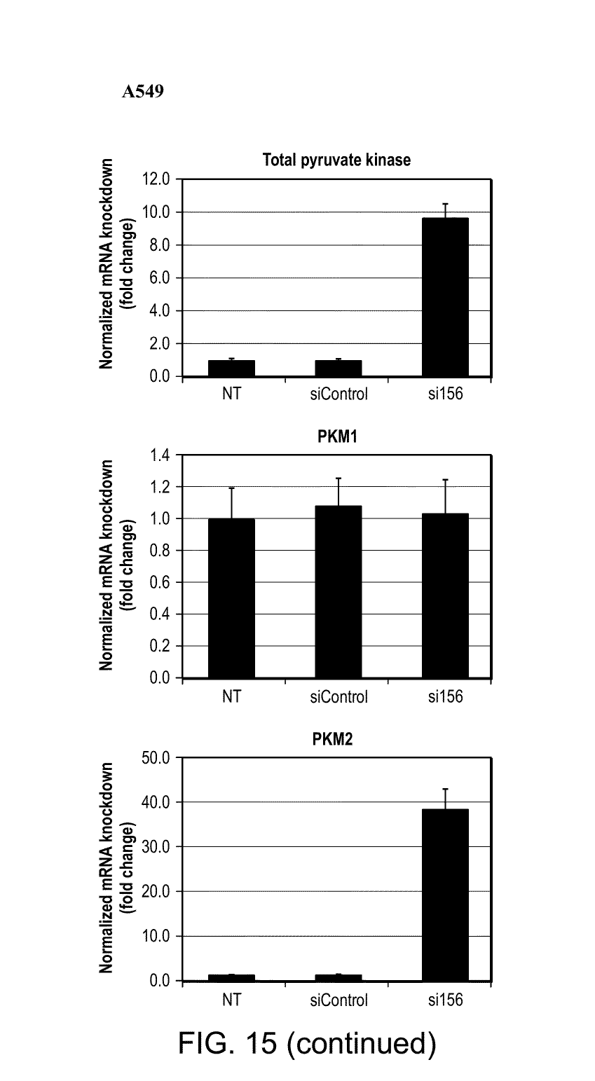

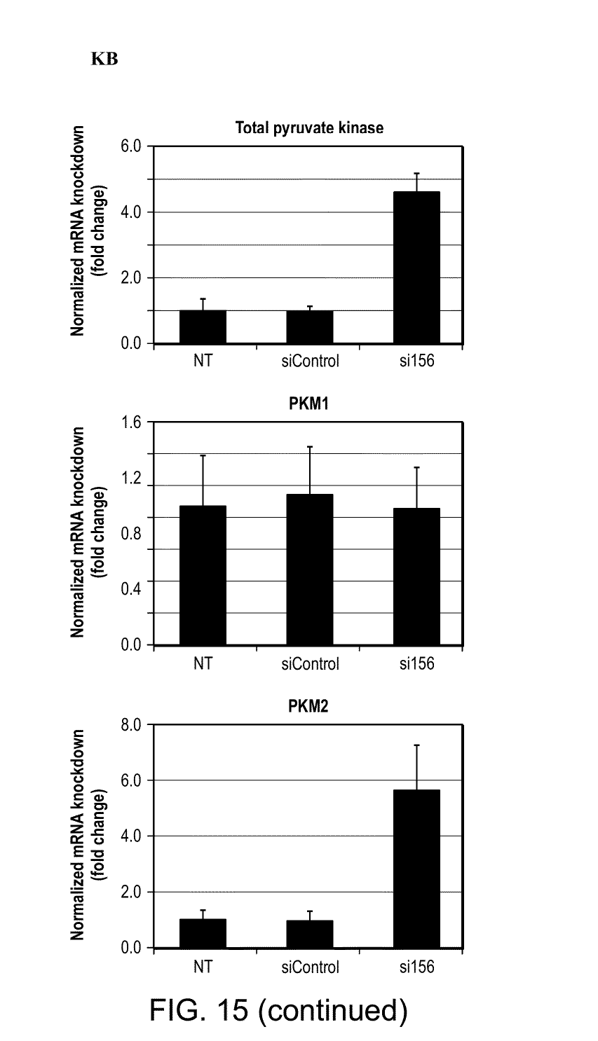

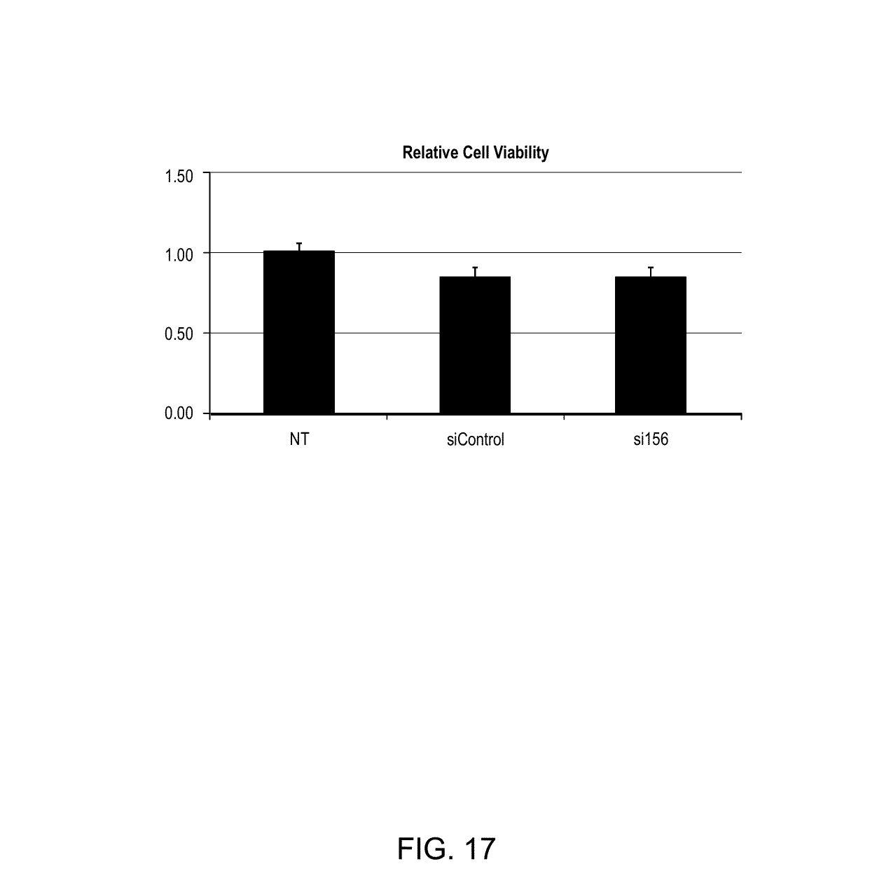

Real-time PCR was used to confirm that the knockdown was robust and specific (FIG. 15). With minimal effect on PKM1 levels, si156 conferred as much as 38-fold knockdown. The specificity of knockdown by si156 was additionally confirmed at the protein level (FIG. 12). Indeed, not only was total PK reduced in HepG2 and SKOV3 cells treated with si156 but also PKM2 was specifically knocked down in both cell lines investigated. Significantly, we confirmed that the effect of si156 on viability is specific to cancer cells (FIG. 17)

### Example 6

### si156 Leads to Profound Regression of Tumor Volume In vivo

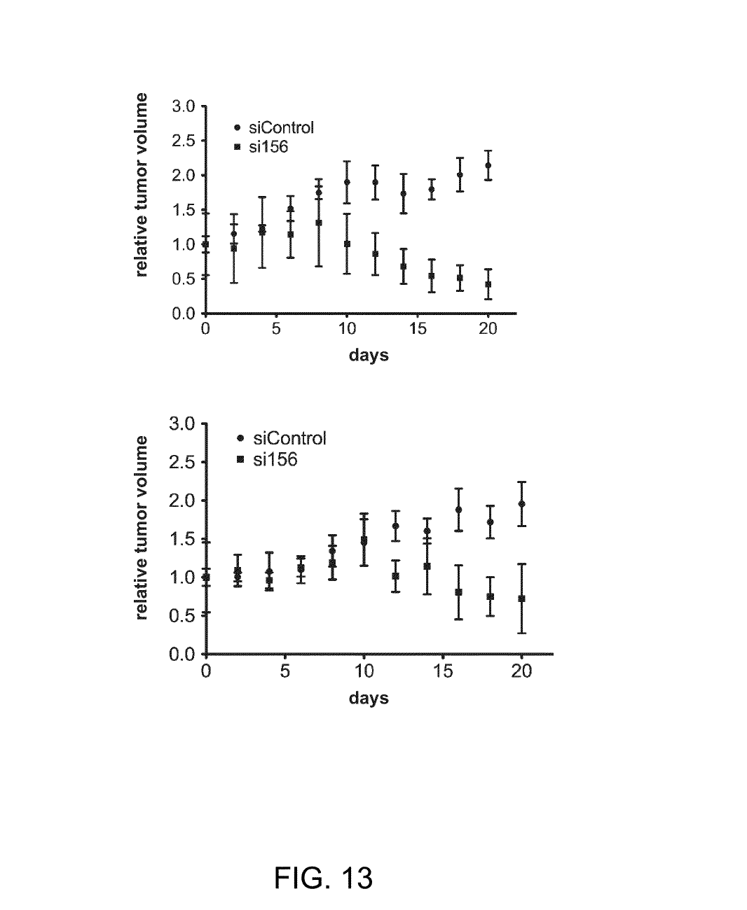

A member of the lipid-like class of materials dubbed “lipidoids”¹² was used to encapsulate and deliver siRNA as a nanoparticle to the xenografts. The selected lipidoid, ND98, has previously been shown to knock down multiple hepatic genes¹² as well as a putative oncogene in vivo, resulting in reduction of tumor volume and extension of survival in two mouse models of ovarian cancer¹³. We evaluated the efficacy of the siRNA treatments in vivo using xenograft models of liver (HepG2) and ovarian (SKOV3) carcinomas that were induced subcutaneously in SCID mice. When the induced tumors were ˜200 mm³ in volume, the mice were randomly divided into separate groups and treated intratumorally twice a week for a period of 20 days with either siControl or si156. Compared to the control treatment, si156 showed robust efficacy against xenograft tumors. Tumor regression was observed after 2-3 doses (˜day 10) for tumors receiving the siPKM2 treatment. The trial was halted when the control group had to be euthanized. At the end of the trial (day 20, 6 treatments), the mean±SD (n=4) tumor volumes were 541±107 mm³ and 83±84 mm³ for HepG2 tumors and 348±102 mm³ and 88±110 mm³ for SKOV3 tumors for siControl and si156, respectively (FIG. 13).

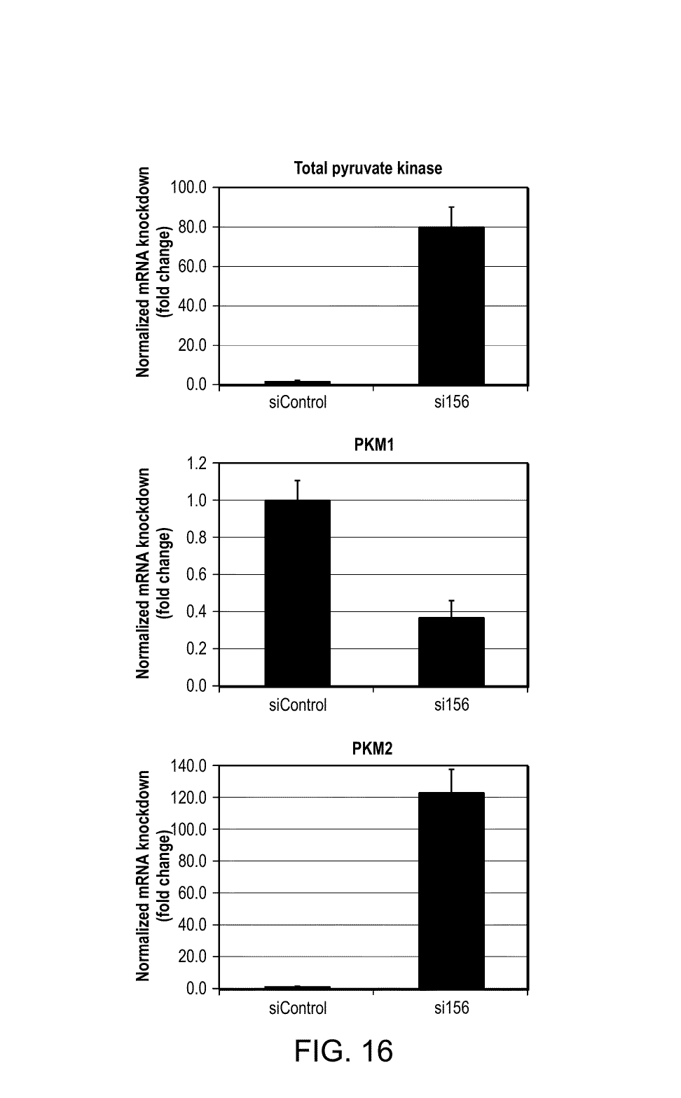

Treatment with si156 prevented tumor expansion and even resulted in dramatic volume reduction. Indeed, nearly all of the measured volume in the treated mice could be attributed to scar tissue caused by the intratumoral injections, as determined by attempted tumor recovery. There was no remnant of HepG2 tumor in half of the si156-treated animals, and there was no remnant of SKOV3 tumor in three quarters of the si156-treated animals. The tumors that were recovered were sectioned and visualized by TUNEL staining. TUNEL staining revealed that si156-treated tumors displayed significantly higher levels of apoptosis as compared to siControl-treated tumors. Apoptosis was virtually undetectable in the siControl-treated tumors, but clearly detected in a substantial portion of stained cells in si156-treated tumors. RNA was extracted from the tumors, and real-time PCR was performed to confirm the specific and dramatic knockdown of PKM2 in the SKOV3 xenograft (FIG. 16). Neither siPKM1 nor siPK was used as a control because both sequences would presumably be toxic to healthy cells.

### Example 7

### Effective Sequences have Few Common Features

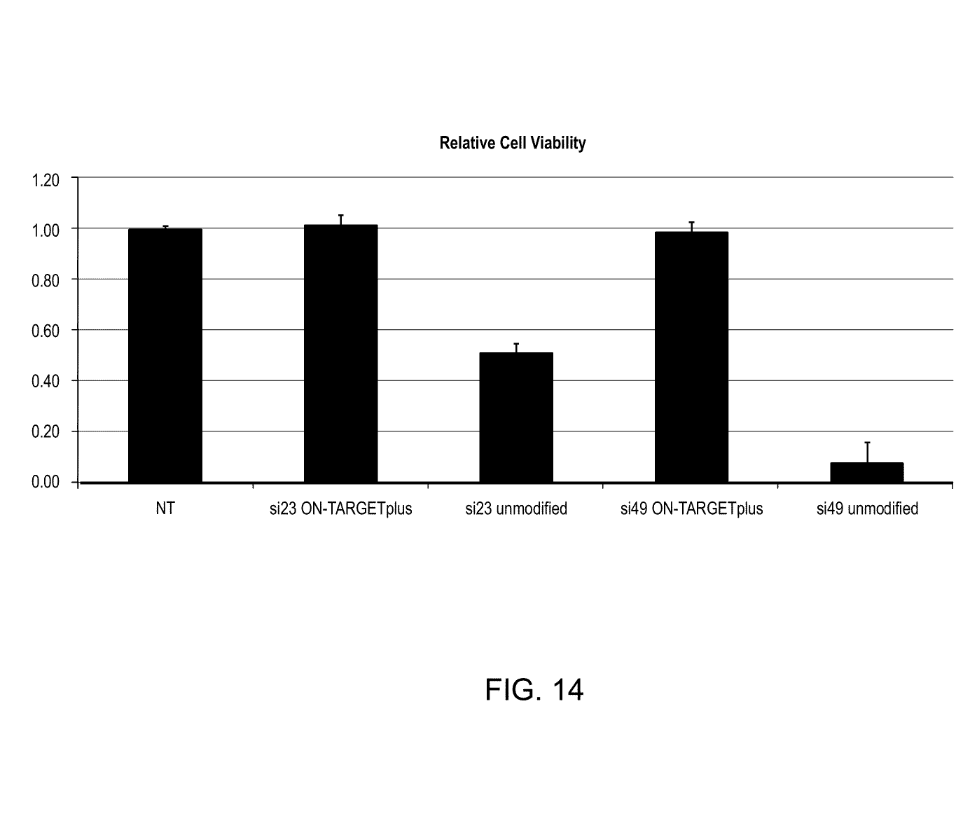

We have identified multiple siRNAs that afford potent and specific silencing of the M2 isoform of pyruvate kinase. An inspection of the 10 top-performing sequences reveals that the number of mismatches between the M1 and M2 isoforms ranges from four to 13. Five of these 10 sequences contain mismatches at one of the central positions 10 and 11, which have been shown to be important in conferring specificity²⁶. Interestingly, nine contain mismatches at one of the termini. There is one “hot spot”: two of the top-three siRNAs target consecutive 19-mers (si155 and si156). There is also one “warm spot”: three siRNAs work moderately well over a sequence that spans 23 nucleotides (si83, si85, and si87). Otherwise, the siRNAs that worked well had few shared features. As mentioned, the importance of the modifications was examined by comparing the viability of cells treated with ON-TARGETplus siRNA or unmodified siRNA of the same sequence (FIG. 14). si23 was chosen because it contains only two mismatches—the minimal number of mismatches within a 19-mer between exons 9 and 10—neither of which is at position 10 and both of which are silent in terms of coding potential. That is, the M1 and M2 isoforms both encode the same amino acid sequence at these residues. No effect was observed for this specific chemically-modified siRNA, whereas a very strong reduction in viability was observed for the unmodified version. This can potentially be explained by the fact that both the sense and antisense strands of si23 contain seed matches for the anti-apoptotic gene Bcl-xL in their 3′UTRs¹⁷. Indeed, in addition to M2-specific knockdown, we observed a marked increase in apoptosis for cells treated with unmodified si23 in multiple cell lines. That a sequence with only two mismatches—neither at thought-to-be critical positions—afforded such strong, specific silencing with concomitant cancer cell death was highly unexpected.

### Example 8

### siPKM2 Represents a Novel Therapeutic Approach for the Treatment of Cancer

While the M2 isoform of pyruvate kinase has not previously been specifically targeted at the mRNA level, it has been previously targeted at the protein level by synthetic peptide aptamers, which specifically bind to PKM2 and shift it into its less active dimeric conformation¹⁸. As expected, the aptamer-induced dimerization led to a significant decrease in the PK mass-action and ATP:ADP ratios, though it has been suggested that the lower activity enhances the cell's ability to use glucose for anabolic processes rather than energy generation. While the peptide aptamers reduced the proliferation rate of immortalized NIH 3T3's and an osteosarcoma cell line, they did so to a modest extent (<10%) and did not affect apoptotic cell death.

The ability to induce cell death, rather than simply retarding growth, which we have demonstrated, is becoming seen as increasingly important for therapies in the clinical setting. Moreover, the peptides were expressed from within the cell; they would likely be degraded in endosomes if they were to be endocytosed, and gene therapy does not appear to be a near-term option for patients. The siRNAs targeting PKM2 described herein represent the first exogenously-administered isoform-specific macromolecules that drug this target.

In addition to its glycolytic role in the cytosol, PKM2, which contains an inducible nuclear translocation signal in its C-domain, functions in the nucleus. Tumor-derived (dimeric) PKM2 can transfer phosphate from PEP to histone H1 rather than to ADP, a process that is stimulated by L-cysteine¹⁸. In contrast to this pro-proliferative role, PKM2 has been shown to translocate to the nucleus in response to apoptotic agents to induce caspase-independent programmed cell death that is independent of its enzymatic activity²⁰. Notably, our results indicate that downregulation of PKM2 protein owing to mRNA degradation in the cytosol results in caspase-dependent programmed cell death.

RNAi offers several advantages over small molecule drugs. First, whereas small molecule drugs act stoichiometrically on their targets, RNAi is a catalytic process. Second, by acting upstream of the amplificative process of translation, RNAi inherently decreases the number of target molecules requiring drugging. As a result, fewer siRNA molecules than small molecules are required to address the underlying disease-causing molecules, and this could translate to fewer side effects for patients. Finally, the inherent complementarity of nucleic acids renders them much more specific than small molecules, which target proteins that often share three-dimensional shapes with other protein family members. This specificity is especially important when targeting a fold as prevalent as the phosphotyrosine-binding domain.

Furthermore, small molecule antagonists of FBP binding by PKM2 may be insufficient to provide a complete abrogation of the role of PKM2 in cancer, as PKM2 has been demonstrated to interact and cooperate with Oct-4 to regulate transcription in a manner that is seemingly unrelated to metabolism²¹. It is notable that the particular transcription factor positively transactivated by PKM2 is one that is known to be important in maintaining pluripotency, thus preventing embryonic cells from differentiating, and has also been implicated in tumorigenesis²². In this study, we have developed a therapeutic strategy that addresses the design criteria of the ideal cancer therapy: addressing a target that is common to all cancers, that is only found in cancers, that can be targeted specifically, and that results in cell death. We demonstrate the first example of specific, potent silencing of the M2-isoform of pyruvate kinase. This knockdown translates to cell death in vitro and tumor regression in vivo. Our findings suggest that selecting and screening the correct sequence is as important as selecting the correct target.

All publications, patents and sequence database entries mentioned herein, including those listed below, are hereby incorporated by reference in their entirety as if each individual publication or patent was specifically and individually indicated to be incorporated by reference. In case of conflict, the present application, including any definitions herein, will control.

Those skilled in the art will recognize, or be able to ascertain using no more than routine experimentation, many equivalents to the specific embodiments described herein.

Articles such as “a,” “an,” and “the”, as used herein and in the claims may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. In some embodiments, exactly one member of the group is present in, employed in, or otherwise relevant to a given product or process. In some embodiments, more than one or all of the group members are present in, employed in, or otherwise relevant to a given product or process. Furthermore, it is to be understood that the invention encompasses all variations, combinations, and permutations in which one or more limitations, elements, clauses, descriptive terms, etc., from one or more of the claims or from relevant portions of the description is introduced into another claim. For example, any claim that is dependent on another claim can be modified to include one or more limitations found in any other claim that is dependent on the same base claim. Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise.

Where elements are presented as lists, e.g., in Markush group format, it is to be understood that each subgroup of the elements is also disclosed, and any element(s) can be removed from the group.

It is also noted that the term “comprising” is intended to be open and permits the inclusion of additional elements or steps. It should be understood that, in general, where the invention, or aspects of the invention, is/are referred to as comprising particular elements, features, steps, etc., certain embodiments of the invention or aspects of the invention consist, or consist essentially of, such elements, features, steps, etc. For purposes of simplicity those embodiments have not been specifically set forth in haec verba herein. Thus for each embodiment of the invention that comprises one or more elements, features, steps, etc., the invention also provides embodiments that consist or consist essentially of those elements, features, steps, etc.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in different embodiments of the invention, to the tenth of the unit of the lower limit of the range, unless the context clearly dictates otherwise. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment of the present invention may be explicitly excluded from any one or more of the claims. Any embodiment, element, feature, application, or aspect of the compositions and/or methods of the invention, can be excluded from any one or more claims. For purposes of brevity, all of the embodiments in which one or more elements, features, purposes, or aspects is excluded are not set forth explicitly herein.

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims.

### REFERENCES

### References

1 DeBerardinis, R. J., Lum, J. J., Hatzivassiliou, G., and Thompson, C. B., The biology of cancer: Metabolic reprogramming fuels cell growth and proliferation. Cell Metabolism 7 (1), 11 (2008).
2 Christofk, H. R. et al., Pyruvate kinase M2 is a phosphotyrosine-binding protein. Nature 452 (7184), 181 (2008).
3 Jurica, M. S. et al., The allosteric regulation of pyruvate kinase by fructose-1,6-bisphosphate. Structure 6 (2), 195 (1998).
4 Noguchi, T., Inoue, H., and Tanaka, T., The M1 and M2-type isozymes of rat pyruvate kinase are produced from the same gene by alternative splicing. J. Biol. Chem. 261 (29), 13807 (1986).
5 Dombrauckas, J. D., Santarsiero, B. D., and Mesecar, A. D., Structural basis for tumor pyruvate kinase M2 allosteric regulation and catalysis. Biochemistry 44 (27), 9417 (2005).
6 Christofk, H. R. et al., The M2 splice isoform of pyruvate kinase is important for cancer metabolism and tumour growth. Nature 452 (7184), 230 (2008).
7 Warburg, O., On the origin of cancer cells. Science 123 (3191), 309 (1956).
8 Ferguson, E. C. and Rathmell, J. C., New roles for pyruvate kinase M2: working out the Warburg effect. Trends in Biochemical Sciences 33 (8), 359 (2008).
9 Ugurel, S. et al., Tumor type M2 pyruvate kinase (TuM2-PK) as a novel plasma tumor marker in melanoma. International Journal of Cancer 117 (5), 825 (2005).
10 Ashizawa, K., Willingham, M. C., Liang, C. M., and Cheng, S. Y., In vivo regulation of monomer-tetramer conversion of pyruvate kinase subtype M2 by glucose is mediated via fructose 1,6-bisphosphate. J. Biol. Chem. 266 (25), 16842 (1991).
11 Bumcrot, David, Manoharan, Muthiah, Koteliansky, Victor, and Sah, Dinah W. Y., RNAi therapeutics: a potential new class of pharmaceutical drugs. Nat Chem Biol 2 (12), 711 (2006).
12 Akinc, Akin et al., A combinatorial library of lipid-like materials for delivery of RNAi therapeutics. Nat Biotech 26 (5), 561 (2008).
13 Huang, Y-H. et al., Claudin-3 gene silencing with siRNA suppresses ovarian tumor growth and metastasis. Proc Natl Acad Sci USA 106 (9), 3426 (2009).
14 Reynolds, Angela et al., Rational siRNA design for RNA interference. Nat Biotech 22 (3), 326 (2004).
15 Boese, Q. et al., Mechanistic insights aid computational short interfering RNA design. Methods in Enzymology 392, 73 (2005).
16 Birmingham, A. et al., A protocol for designing siRNAs with high functionality and specificity. Nat. Protocols 2 (9), 2068 (2007).
17 Birmingham, A. et al., 3′ UTR seed matches, but not overall identity, are associated with RNAi off-targets. Nat Meth 3 (3), 199 (2006).
18 Spoden, G. A. et al., Isotype-specific inhibitors of the glycolytic key regulator pyruvate kinase subtype M2 moderately decelerate tumor cell proliferation. International Journal of Cancer 123 (2), 312 (2008).
19 Ignacak, J. and Stachurska, M. B., The dual activity of pyruvate kinase type M2 from chromatin extracts of neoplastic cells. Comp Biochem Physiol B Biochem Mol Biol 134 (3), 425 (2003).
20 Stetak, A. et al., Nuclear translocation of the tumor marker pyruvate kinase M2 induces programmed cell death. Cancer Res 67 (4), 1602 (2007).
21 Lee, J., Kim, H. K., Han, Y-M., and Kim, J., Pyruvate kinase isozyme type M2 (PKM2) interacts and cooperates with Oct-4 in regulating transcription. The International Journal of Biochemistry & Cell Biology 40 (5), 1043 (2008).
22 Hochedlinger, K., Yamada, Y., Beard, C., and Jaenisch, R., Ectopic expression of Oct-4 blocks progenitor-cell differentiation and causes dysplasia in epithelial tissues. Cell 121 (3), 465 (2005).
23. David, C. J. et al., HnRNP proteins controlled by c-Myc deregulate pyruvate kinase mRNA splicing in cancer. Nature 463 (7279), 364 (2009).
24. Vander Heiden, M. G. et al., Identification of small molecule inhibitors of pyruvate kinase M2. Biochemical Pharmacology Epub ahead of print (2009); Boxer, M. B. et al., Evaluation of substituted N,N′-diarylsulfonamides as activators of the tumor cell specific M2 isoform of pyruvate kinase. Journal of Medicinal Chemistry Epub ahead of print (2009).
25. Love, K. T. et al., Lipid-like materials for low-dose, in vivo gene silencing. Proceedings of the National Academies of Sciences Epub ahead of print (2010); Semple, S. C. et al., Rational design of cationic lipids for siRNA delivery. Nature Biotechnology Epub ahead of print (2010).
26 Wang, Y. et al., Structure of the guide-strand-containing argonaute silencing complex. Nature 456 (7219), 209 (2008).
27. Schwarz, D. S. et al., Designing siRNA that distinguish between genes that differ by a single nucleotide. PLos Genetics 2 (9), e140 (2006).

1. A composition comprising a nucleic acid molecule, wherein the nucleic acid molecule comprises at least 14 contiguous nucleotides of any of the structures given in SEQ ID NOs 15-48.

2. The composition of claim 1, wherein the nucleic acid molecule comprises a sense strand given in any of SEQ ID NOs 68, 94, 99, 107, 119, 125, 145, 150, 152, 154, 189, 213, 222, or 223.

3. The composition of claim 1, wherein the nucleic acid molecule comprises the siRNA sense strand of any of si1, si27, si32, si40, si52, si58, si78, si83, si85, si87, si122, si146, si155, or si156.

4. The composition of claim 1, wherein the nucleic acid molecule consists of the siRNA sense strand of any of si1, si27, si32, si40, si52, si58, si78, si83, si85, si87, si122, si146, si155, or si156.

5. The composition of claim 1, wherein the nucleic acid molecule further comprises at least one U nucleotide appended at the 3′-end.

6. The composition of claim 1, wherein the nucleic acid molecule is 19-21 nucleotides in length.

7. The composition of claim 1, wherein the nucleic acid molecule is 22-27 nucleotides in length.

8. The composition of claim 1, wherein the nucleic acid molecule is 28-49 nucleotides in length.

9. A double-stranded nucleic acid molecule comprising an antisense strand that is the nucleic acid molecule of claim 1, and a sense strand complementary to the antisense strand.

10. The composition of claim 1, wherein the nucleic acid molecule is an RNA molecule.

11. A composition comprising a nucleic acid molecule complementary to the structure given in any of SEQ ID NOs 1-14.

12. A small interfering RNA (siRNA) specifically targeting isoform M2 of pyruvate kinase (PKM2), the siRNA comprising a duplex stem region, wherein the duplex stem region comprises a nucleotide sequence corresponding to a target sequence of exon 10 of pyruvate kinase (PK), and wherein the target sequence is selected from the group of structures given in SEQ ID NOs 1-14.

13. The siRNA of claim 12, wherein the siRNA does not substantially inhibit the expression of isoform M1 of pyruvate kinase (PKM1).

14. The siRNA of claim 12, wherein the pyruvate kinase (PK) is human PK.

15. The siRNA of claim 12, wherein the siRNA target sequence is between 12 and 49 nucleotides long.

16. The siRNA of claim 12, wherein the siRNA target sequence is between 25 and 30 nucleotides long.

17. The siRNA of claim 12, wherein the siRNA target sequence is between 19 and 26 nucleotides long.

18. The siRNA of claim 12, wherein the siRNA is 19-22 nucleotides long.

19. The siRNA of claim 12, wherein the siRNA comprises a 3′ overhang.

20. A pharmaceutical composition comprising the siRNA of claim 12.

21. A method of decreasing expression of PKM2 in a cell, comprising
contacting a cell expressing PKM2 with a PKM2-inhibitory nucleic acid molecule specifically targeting PKM2, wherein the PKM2-inhibitory nucleic acid molecule does not substantially inhibit the expression of PKM1.

22. The method of claim 21, wherein the PK is human PK.

23. The method of claim 21, wherein the PKM2-inhibitory nucleic acid molecule targets a sequence within exon 10 of PK.

24. The method of claim 21, wherein the PKM2-inhibitory nucleic acid molecule target sequence is between 12 and 40 nucleotides long.

25. The method of claim 21, wherein the PKM2-inhibitory nucleic acid molecule comprises a nucleotide sequence corresponding to a target sequence selected from the group of structures given in SEQ ID NOs 1-14.

26. The method of claim 21, wherein the PKM2-inhibitory nucleic acid molecule is a siRNA.

27. A method of treating a subject having a tumor expressing PKM2 or suspected to express PKM2, comprising administering to the subject having the tumor a PKM2-inhibitory nucleic acid molecule specifically targeting PKM2, wherein the PKM2-inhibitory nucleic acid molecule does not substantially inhibit the expression of PKM1.

28. The method of claim 27, wherein the PK is human PK.

29. The method of claim 27, wherein the PKM2-inhibitory nucleic acid molecule targets a sequence within exon 10 of PK.

30. The method of claim 27, wherein the PKM2-inhibitory nucleic acid molecule target sequence is between 12 and 40 nucleotides long.

31. The method of claim 27, wherein the PKM2-inhibitory nucleic acid molecule comprises a nucleotide sequence corresponding to a target sequence selected from the group consisting of structures given in SEQ ID NOs 1-14.

32. The method of claim 27, wherein the PKM2-inhibitory nucleic acid molecule is a siRNA.
